(19) United States
(12) Patent Application Publication
(10) Pub. No.: US 2024/0287162 A1
(43) Pub. Date: Aug. 29, 2024

(54) MULTIVALENT ANTI-SPIKE PROTEIN BINDING MOLECULES AND USES THEREOF

(71) Applicant: Regeneron Pharmaceuticals, Inc., Tarrytown, NY (US)

(72) Inventors: Nianshuang WANG, Cos Cob, CT (US); Yang SHEN, Scarsdale, NY (US); Alina BAUM, Pleasantville, NY (US); Christos KYRATSOUS, Irvington, NY (US); Chia-Yang LIN, Scarsdale, NY (US); Tri NGUYEN, Dobbs Ferry, NY (US)

(73) Assignee: Regeneron Pharmaceuticals, Inc., Tarrytown, NY (US)

(21) Appl. No.: 18/589,364

(22) Filed: Feb. 27, 2024

Related U.S. Application Data
(60) Provisional application No. 63/487,424, filed on Feb. 28, 2023.

(51) Int. Cl. C07K 16/10; A61P 31/14

(52) U.S. Cl. CPC C07K 16/1003; A61P 31/14; C07K 2317/21; C07K 2317/24; C07K 2317/31; C07K 2317/35

(57) ABSTRACT

The present disclosure provides multivalent anti-spike protein binding molecules. The present disclosure further relates to the methods of producing the multivalent anti-spike protein binding molecules, pharmaceutical compositions comprising of the multivalent anti-spike protein binding molecules, and methods of use of the multivalent anti-spike protein binding molecules, e.g., to treat conditions associated with SARS-CoV and SARS-CoV-2 infections, such as COVID-19.

### 1. CROSS-REFERENCE TO RELATED APPLICATIONS

This application claims the priority benefit of U.S. provisional application No. 63/487,424, filed Feb. 28, 2023, the contents of which are incorporated herein in their entirety by reference thereto.

### 2. SEQUENCE LISTING

The instant application contains a Sequence Listing which has been submitted electronically in XML format and is hereby incorporated by reference in its entirety. Said XML Sequence Listing, created on Feb. 27, 2024, is named RGN-029US_SL.xml and is 641,288 bytes in size.

### 3. BACKGROUND

Severe acute respiratory syndrome coronavirus 2 (SARS-CoV-2) is an enveloped, positive-sense, single-stranded RNA virus of the genus Betacoronavirus, which also includes SARS-CoV, Middle East respiratory syndrome coronavirus (MERS-CoV), human coronavirus (HCoV)-OC43, and HCoV-HKU1 (Jackson et al., 2022, Nat Rev Mol Cell Biol. 23(1): 3-20). SARS-CoV-2 causes COVID-19, a potentially life-threatening disease which was first characterized in late 2019 and escalated into a global pandemic in early 2020.

SARS-CoV-2 shares ˜80% identity with SARS-CoV and both viruses rely on their interaction with the angiotensin-converting enzyme 2 (ACE2) for cellular entry, an enzyme expressed on the extracellular surface of many types of cells.

Currently, several vaccines against SARS-CoV-2 are used to prevent manifestation of severe disease. However, vaccination rates vary among populations, and even in areas with high rates of vaccination, breakthrough infections leading to COVID-19 have been observed in individuals who have been immunized against SARS-CoV-2. Previous SARS-CoV-2 infections don't seem to provide a complete immunity against future infections either, as some individuals have been diagnosed with COVID-19 multiple times. Moreover, SARS-CoV-2 infection in some individuals leads to a prolonged disease associated with persistence of one or more symptoms of COVID-19 for weeks to months after the clearance of infection. These observations underscore the serious population health threat posed by COVID-19 and the need to fight SARS-CoV-2 infections with effective treatments.

Small molecule treatments such as Paxlovid—a combination of the oral antiviral drugs nirmatrelvir and ritonavir—can prevent hospitalization, yet they are associated with a ‘Paxlovid rebound’ effect in which the virus reemerges (Callaway, Nature (News), 11 Aug. 2022). On the other hand, biologic treatments, such as monoclonal antibodies, can directly blunt viral propagation, leading to robust and long-lasting therapeutic effects. However, due to rapid emergence of new SARS-CoV-2 variants, antibodies isolated from patients are typically strain-specific, rendering them ineffective against certain SARS-CoV-2 variants. Hence, there remains a need to develop neutralizing treatments that will be effective against SARS-CoV-2.

### 4. SUMMARY

The present disclosure relates to multivalent antigen-binding molecules that bind to a coronavirus spike protein, generally referred to herein as “multivalent anti-spike protein binding molecules”, suitable for inhibiting the interaction between coronaviruses and host cells. Multivalent anti-spike protein binding molecules of the disclosure typically comprise a plurality of spike protein antigen-binding domains (ABDs) (e.g., ten or twelve spike protein ABDs) operably linked by one or more multimerization moieties (e.g., five or six multimerization moieties). Multivalent anti-spike protein binding molecules of the disclosure are described in numbered embodiments 1 to 102.

The multivalent anti-spike protein binding molecules of the disclosure are typically decavalent or dodecavalent and comprise ABDs, e.g., in the form of a Fab or an scFv. In some embodiments, ten or twelve ABDs are spike protein ABDs. The multivalent anti-spike protein binding molecule of the disclosure can be monospecific (e.g., all ABDs bind to the same region of spike protein and optionally all have the same sequence) or multispecific (e.g., at least two of the ABDs bind to different regions or variants of spike protein and differ in sequence, or bind to spike protein and another target). Spike protein ABDs and spike protein ABD formats that are suitable for incorporation into multivalent anti-spike protein binding molecules of the disclosure are described in Sections 6.2 and 6.3, and in numbered embodiments 4 to 27, 36, and 37.

The multivalent anti-spike protein binding molecules of the disclosure include one or more multimerization moieties. Typically, a multivalent anti-spike protein binding molecule of the disclosure comprises five or six multimerization moieties that each comprise or consist of an Fc dimer, e.g., five or six dimeric IgM Fc domains. In certain aspects, the multivalent anti-spike protein binding molecule further comprises a J chain connecting the IgM Fc domains, whose inclusion typically provides a molecule comprising ten ABDs (vs twelve ABDs in a molecule without a J chain connecting the IgM Fc domains). In some embodiments, the J-chain is operably linked to an Fc domain (which can be a dimerizing IgG domain, a non-dimerizing Fc domain, or an Fc 1.5 domain), for example an IgG Fc domain. Multimerization moieties suitable for incorporation into the multivalent anti-spike protein binding molecules of the disclosure are described in Section 6.4 and numbered embodiments 35, and 38 to 66.

Two or more components of the multivalent anti-spike binding protein binding molecules of the disclosure can be connected to one another by a linker, e.g., a peptide linker. By way of example and without limitation, linkers can be used to connect a spike protein ABD to a multimerization moiety. Linkers suitable for incorporation into the multivalent anti-spike binding protein binding molecules of the disclosure are described in Section 6.5.

The present disclosure further provides nucleic acids encoding the multivalent anti-spike protein binding molecules of the disclosure, host cells engineered to express the multivalent anti-spike protein binding molecules of the disclosure, and recombinant methods for the production of the multivalent anti-spike protein binding molecules of the disclosure. Such nucleic acids, host cells and production methods are described in Section 6.6 and numbered embodiments 103 to 105.

The present disclosure further provides pharmaceutical compositions comprising the multivalent anti-spike protein binding molecules of the disclosure as well as therapeutic indications and methods of use. Pharmaceutical compositions are described in Section 6.7 and numbered embodiment 106. Methods of use of the multivalent anti-spike protein binding molecules are described in Section 6.8 and numbered embodiments 107 to 119.

Other features and advantages of aspects of the multivalent anti-spike protein binding molecules of the present disclosure will become apparent from the following more detailed description, taken in conjunction with the accompanying drawings.

### 5. BRIEF DESCRIPTION OF THE FIGURES

FIGS. 1A-1E show the structures of exemplary IgM alternative format (“AF”) antibody pentameric and hexameric constructs of the disclosure, where:
(1) represents a Fab moiety;
(2) represents a Cμ2 domain of IgM-Fc;
(3) represents a Cμ3 domain of IgM-Fc;
(4) represents a Cμ4 domain of IgM-Fc;
(5) represents a J chain region of an IgM;
(6) represents a J chain operably linked to a dimerizing IgG Fc domain;
(7) represents a J chain operably linked to an Fc1.5 domain;
(8) represents a J chain operably linked to a non-dimerizing Fc domain.

FIG. 2 shows two representative reducing SDS-PAGE gels of six exemplary IgM constructs with distinct anti-SARS-CoV-2 S-protein Fab moieties, before (left gel) or after (right gel) affinity purification.

FIGS. 3A-3F show the size exclusion chromatography (SEC) profiles of six exemplary IgM constructs with distinct anti-SARS-CoV-2 S-protein Fab moieties. FIG. 3A shows the SEC profile of the IgM construct, 10933-IgM. FIG. 3B shows the SEC profile of the IgM construct, 14256-IgM. FIG. 3C shows the SEC profile of the IgM construct, 10987-IgM. FIG. 3D shows the SEC profile of the IgM construct, 14315-IgM. FIG. 3E shows the SEC profile of the IgM construct, 10985-IgM. FIG. 3F shows the SEC profile of the IgM construct, 10989-IgM.

FIGS. 4A-4C show the extent of neutralization of SARS-CoV-2 pseudovirus D614G and BA. 1 and BA.2 variants by different RBD-targeting exemplary IgMs. FIG. 4A shows the extent of neutralization of the pseudovirus, D614G. FIGS. 4B and 4C show the extent of neutralization of BA. 1 and BA.2 variants, respectively. REGEN-COV (REGN10987/REGN10933) was included as a control in all assessments.

FIGS. 5A-5C show the neutralization potency of 10933-IgM against SARS-CoV-2 pseudovirus D614G and two Omicron variants. REGN10933 parental IgG and REGEN-COV (REGN10987/REGN10933) were also included for comparison. FIG. 5A shows the extent of neutralization of D614G. FIG. 5B shows the extent of neutralization of the BA. 1 variant, and FIG. 5C shows the extent of neutralization of the BA.2 variant.

FIGS. 6A-6C show the neutralization potency of 10989-IgM against SARS-CoV-2 pseudovirus D614G and two Omicron variants. REGN10989 parental IgG and REGEN-COV (REGN10987/REGN10933) were also included for comparison. FIG. 6A shows the extent of neutralization of D614G. FIG. 6B shows the extent of neutralization of the BA. 1 variant, and FIG. 6C shows the extent of neutralization of the BA.2 variant.

FIGS. 7A-7C show the neutralization potency of 10987-IgM against SARS-CoV-2 pseudovirus D614G and two Omicron variants. REGN10987 parental IgG and REGEN-COV (REGN10987/REGN10933) were also included for comparison. FIG. 7A shows the extent of neutralization of D614G. FIG. 7B shows the extent of neutralization of the BA. 1 variant, and FIG. 7C shows the extent of neutralization of the BA.2 variant.

FIGS. 8A-8C show the neutralization potency of 10985-IgM against SARS-CoV-2 pseudovirus D614G and two Omicron variants. REGN10985 parental IgG and REGEN-COV (REGN10987/REGN10933) were also included for comparison. FIG. 8A shows the extent of neutralization of D614G. FIG. 8B shows the extent of neutralization of the BA. 1 variant, and FIG. 8C shows the extent of neutralization of the BA.2 variant.

FIGS. 9A-9C show the neutralization potency of 14315-IgM against SARS-CoV-2 pseudovirus D614G and two Omicron variants. REGN14315 parental IgG and REGEN-COV (REGN10987/REGN10933) were also included for comparison. FIG. 9A shows the extent of neutralization of D614G. FIG. 9B shows the extent of neutralization of the BA. 1 variant, and FIG. 9C shows the extent of neutralization of the BA.2 variant.

FIGS. 10A-10E show the neutralization potency of 14287-IgM against SARS-CoV-2 pseudovirus D614G and four Omicron variants. REGN14287 parental IgG and REGEN-COV (REGN10987/REGN10933) were also included for comparison. FIG. 10A shows the extent of neutralization of D614G. FIG. 10B shows the extent of neutralization of the BA. 1 variant. FIG. 10C shows the extent of neutralization of the BA.2 variant. FIG. 10D shows the extent of neutralization of the BA.2.75 variant. FIG. 10E shows the extent of neutralization of the BA.4/BA.5 variant.

FIGS. 11A-11C show the neutralization potency of 14315 and 14287-IgMs against currently circulating Omicron variant BQ. 1. FIG. 11A shows neutralization of BQ. 1 by 14315-IgM, 14287-IgM, 10933-IgM, and 10985-IgM. REGEN-COV was included as a control. FIG. 11B shows the BQ. 1 neutralization comparison between 14315-IgM and REGN14315 IgG. FIG. 11C shows the BQ. 1 neutralization comparison between 14287-IgM and REGN14287 IgG.

FIGS. 12A-12B show Protein A binding and size exclusion chromatography (SEC) of multivalent anti-spike protein binding molecules comprising IgG Fc-linked J chains. FIG. 12A shows a representative SDS-PAGE gels of three exemplary IgM constructs after Protein A incubation. FIG. 12B shows the SEC profiles of the same three exemplary IgM constructs shown in FIG. 12A.

FIGS. 13A-13B show the neutralization potency of 14287-IgMs comprising IgG Fc-linked J chains against SARS-CoV-2 pseudovirus D614G and XBB1.5 variant. REGN14287 parental IgG, 14287-IgM, and REGEN-COV (REGN10987/REGN10933) were also included for comparison. FIG. 13A shows the extent of neutralization of D614G. FIG. 13B shows the extent of neutralization of the XBB1.5 variant.

### 6. DETAILED DESCRIPTION

### 6.1. Definitions

As used herein, the following terms are intended to have the following meanings:

About, Approximately: The terms “about”, “approximately” and the like are used throughout the specification in front of a number to show that the number is not necessarily exact (e.g., to account for fractions, variations in measurement accuracy and/or precision, timing, etc.). It should be understood that a disclosure of “about X” or “approximately X” where X is a number is also a disclosure of “X.” Thus, for example, a disclosure of an embodiment in which one sequence has “about X % sequence identity” to another sequence is also a disclosure of an embodiment in which the sequence has “X % sequence identity” to the other sequence.

And, or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

Antibody: The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain comprises a heavy chain variable region (abbreviated herein as VH) and a heavy chain constant region. The heavy chain constant region comprises three domains, CH1, CH2 and CH3. Each light chain comprises a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, but is not limited to, monoclonal antibodies, human antibodies, humanized antibodies, camelized antibodies, chimeric antibodies, bispecific or multispecific antibodies and anti-idiotypic (anti-id) antibodies. The antibodies can be of any isotype/class (e.g., IgG, IgE, IgM, IgD, IgA and IgY) or subclass (e.g., IgG1, IgG2, IgG3, IgG4, IgA1 and IgA2). Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding domain or amino-terminus of the antibody. The N-terminus is a variable region and at the C-terminus is a constant region; the CH3 and CL domains represent the carboxy-terminus of the heavy and light chain, respectively, of natural antibodies. For convenience, and unless the context dictates otherwise, the reference to an antibody also refers to antibody fragments as well as engineered antibodies that include non-naturally occurring antigen-binding domains and/or antigen-binding domains having non-native configurations.

Antigen Binding Molecule or ABM: The term “antigen binding molecule” or “ABM” as used herein refers to a molecule (e.g., an assembly of multiple polypeptide chains) comprising two half antibodies. Typically, each half antibody comprises at least one antigen-binding domain. In some embodiments, the antigen is a coronavirus spike protein; hence, the ABMs of the disclosure are generally referred to as “anti-spike protein binding molecules.” The ABMs of the disclosure can be monospecific or multispecific (e.g., bispecific). The antigen binding domain in monospecific binding molecules all bind to the same epitope whereas multispecific binding molecules have at least two antigen-binding sites that bind to different epitopes, which can be on the same or different molecules (e.g., different spike protein variants).

Antigen-binding domain: The term “antigen-binding domain” or “ABD” as used herein refers to a portion of an antibody or antibody fragment that has the ability to bind to an antigen non-covalently, reversibly and specifically. Examples of an antibody fragment that can comprise an ABD include, but are not limited to, a single-chain Fv (scFv), a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989, Nature 341:544-546), which consists of a VH domain; and an isolated complementarity determining region (CDR). Thus, the term “antibody fragment” encompasses both proteolytic fragments of antibodies (e.g., Fab and F(ab)₂ fragments) and engineered proteins comprising one or more portions of an antibody (e.g., an scFv). Antibody fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology 23: 1126-1136).

Associated: The term “associated” in the context of a multivalent anti-spike protein binding molecule refers to a functional relationship between two or more polypeptide chains. In particular, the term “associated” means that two or more polypeptides are associated with one another, e.g., non-covalently through molecular interactions or covalently through one or more disulfide bridges or chemical cross-linkages, so as to produce a functional multivalent anti-spike protein binding molecule. Examples of associations that might be present in a multivalent anti-spike protein binding molecule of the disclosure include (but are not limited to) associations between Fc domains to form an Fc region (e.g., as described in Section 6.4.1).

Bispecific: The term “bispecific” as used herein refers to antigen-binding molecules comprising two or more different ABDs. For instance, ABDs in a bispecific molecule can bind to two different portions of the same target antigen (or, in the case of a viral protein, different variants of the same target antigen) or each ABD can bind to a different target antigen.

Bivalent: The term “bivalent” as used herein refers to a binding molecule comprising two antigen binding domains, whether in the same polypeptide chain or on different polypeptide chains.

Complementarity Determining Region: The terms “complementarity determining region” or “CDR,” as used herein, refer to the sequences of amino acids within antibody variable regions which confer antigen specificity and binding affinity. For example, in general, there are three CDRs in each heavy chain variable region (e.g., CDR-H1, CDR-H2, and CDR-H3) and three CDRs in each light chain variable region (CDR-L1, CDR-L2, and CDR-L3). The precise amino acid sequence boundaries of a given CDR can be determined using any of a number of well-known schemes, including those described by Kabat et al., 1991, “Sequences of Proteins of Immunological Interest,” 5th Ed. Public Health Service, National Institutes of Health, Bethesda, MD (“Kabat” numbering scheme), Al-Lazikani et al., 1997, JMB 273:927-948 (“Chothia” numbering scheme) and ImMunoGenTics (IMGT) numbering (Lefranc, 1999, The Immunologist 7:132-136; Lefranc et al., 2003, Dev. Comp. Immunol. 27:55-77 (“IMGT” numbering scheme). For example, for classic formats, under Kabat, the CDR amino acid residues in the heavy chain variable domain (VH) are numbered 31-35 (CDR-H1), 50-65 (CDR-H2), and 95-102 (CDR-H3); and the CDR amino acid residues in the light chain variable domain (VL) are numbered 24-34 (CDR-L1), 50-56 (CDR-L2), and 89-97 (CDR-L3). Under Chothia, the CDR amino acids in the VH are numbered 26-32 (CDR-H1), 52-56 (CDR-H2), and 95-102 (CDR-H3); and the amino acid residues in VL are numbered 26-32 (CDR-L1), 50-52 (CDR-L2), and 91-96 (CDR-L3). By combining the CDR definitions of both Kabat and Chothia, the CDRs consist of amino acid residues 26-35 (CDR-H1), 50-65 (CDR-H2), and 95-102 (CDR-H3) in human VH and amino acid residues 24-34 (CDR-L1), 50-56 (CDR-L2), and 89-97 (CDR-L3) in human VL. Under IMGT the CDR amino acid residues in the VH are numbered approximately 26-35 (CDR-H1), 51-57 (CDR-H2) and 93-102 (CDR-H3), and the CDR amino acid residues in the VL are numbered approximately 27-32 (CDR-L1), 50-52 (CDR-L2), and 89-97 (CDR-L3) (numbering according to “Kabat”). Under IMGT, the CDR regions of an antibody can be determined using the program IMGT/DomainGap Align.

COVID-19: The term “COVID-19” is the abbreviation of “Coronavirus disease 2019” and refers to the infectious disease caused by SARS-CoV-2 infection. Patients with COVID-19 may experience a wide range of symptoms ranging from mild to severe, which may include but are not limited to, fever, chills, cough, shortness of breath, difficulty breathing, fatigue, muscle aches, body aches, headache, loss of smell, loss of taste, sore throat, congestion, runny nose, nausea, and diarrhea.

Decavalent: The term “decavalent” as used herein in relation to an antigen-binding molecule comprising ten ABDs. The decavalent anti-spike protein binding molecules of the disclosure can be monospecific or multispecific, e.g., bispecific. In some embodiments, a decavalent anti-spike protein binding molecule refers to an anti-spike protein binding molecule comprising ten spike protein ABDs. The ten spike protein ABDs can be the same (i.e., the antigen binding molecule is monospecific) or different (i.e., the antigen binding molecule is multispecific and binds to different regions and/or variants of spike protein). In some embodiments, a decavalent anti-spike protein binding molecule is a pentameric assembly of five IgM Fc dimers, with each IgM Fc comprising a spike protein ABD at its N-terminus, connected via a J chain. In other embodiments, a decavalent anti-spike protein binding molecule refers to an anti-spike protein comprising a plurality of spike protein ABDs and a plurality of ABDs that bind to another target molecule, i.e., the antigen binding molecules are multispecific. For example, in some embodiments, the decavalent anti-spike protein binding molecule is bispecific and comprises five anti-spike protein ABDs and five ABDs that bind to another target.

Dodecavalent: The term “dodecavalent” as used herein in relation to an antigen-binding molecule comprising twelve ABDs. The dodecavalent anti-spike protein binding molecules of the disclosure can be monospecific or multispecific, e.g., bispecific. In some embodiments, a dodecavalent anti-spike protein binding molecule refers to an anti-spike protein binding molecule comprising twelve spike protein ABDs. The twelve spike protein ABDs can be the same (i.e., the antigen binding molecule is monospecific) or different (i.e., the antigen binding molecule is multispecific and binds to different regions and/or variants of spike protein). In some embodiments, a dodecavalent spike protein binding molecule is a hexameric assembly of six IgM Fc dimers, with each IgM Fc comprising a spike protein ABD at its N-terminus, without a J chain connection. In other embodiments, a dodecavalent anti-spike protein binding molecule refers to an anti-spike protein comprising a plurality of spike protein ABDs and a plurality of ABDs that bind to another target molecule, i.e., the antigen binding molecules are multispecific. For example, in some embodiments, the dodecavalent anti-spike protein binding molecule is bispecific and comprises six anti-spike protein ABDs and six ABDs that bind to another target.

EC50: The term “EC50” refers to the half maximal effective concentration of a molecule (such as a multivalent anti-spike protein binding molecule) which induces a response halfway between the baseline and maximum after a specified exposure time. The EC50 essentially represents the concentration of a multivalent anti-spike protein binding molecule where 50% of its maximal effect is observed. In certain embodiments, the EC50 value equals the concentration of a multivalent anti-spike protein binding molecule that gives half-maximal virus or pseudovirus neutralization in an assay as described in Section 8.1.2.

Epitope: An epitope, or antigenic determinant, is a portion of an antigen recognized by an antibody or a fragment thereof, e.g., an antigen-binding domain. An epitope can be linear or conformational.

Fab: The term “Fab” refers to a pair of polypeptide chains, the first comprising a variable heavy (VH) domain of an antibody operably linked (typically N-terminal to) to a first constant domain (referred to herein as C1), and the second comprising variable light (VL) domain of an antibody N-terminal operably linked (typically N-terminal) to a second constant domain (referred to herein as C2) capable of pairing with the first constant domain. In a native antibody, the VH is N-terminal to the first constant domain (CH1) of the heavy chain and the VL is N-terminal to the constant domain of the light chain (CL). The Fabs of the disclosure can be arranged according to the native orientation or include domain substitutions or swaps that facilitate correct VH and VL pairings. For example, it is possible to replace the CH1 and CL domain pair in a Fab with a CH3-domain pair to facilitate correct modified Fab-chain pairing in heterodimeric molecules. It is also possible to reverse CH1 and CL, so that the CH1 is attached to VL and CL is attached to the VH, a configuration generally known as Crossmab. The term “Fab” encompasses single chain Fabs.

Fc Domain and Fc Region: The term “Fc domain” refers to a portion of the heavy chain that pairs with the corresponding portion of another heavy chain. In some embodiments an Fc domain comprises a CH2 domain followed by a CH3 domain, with or without a hinge region N-terminal to the CH2 domain. The term “Fc region” refers to the region formed by association of two heavy chain Fc domains. The two Fc domains within the Fc region may be the same or different from one another. In a native antibody the Fc domains are typically identical, but one or both Fc domains might be modified to allow for heterodimerization, e.g., via a knob-in-hole interaction.

Fv: The term “Fv” refers to the minimum antibody fragment derivable from an immunoglobulin that contains a complete target recognition and binding site. This region consists of a dimer of one heavy and one light chain variable domain in a tight, noncovalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind target. The reference to a VH-VL dimer herein is not intended to convey any particular configuration. When present on a single polypeptide chain (e.g., a scFv), the VH and be N-terminal or C-terminal to the VL.

Host cell: The term “host cell” as used herein refers to cells into which a nucleic acid of the disclosure has been introduced. The terms “host cell” and “recombinant host cell” are used interchangeably herein. It is understood that such terms refer to the particular subject cell and to the progeny or potential progeny of such a cell. Because certain modifications may occur in succeeding generations due to either mutation or environmental influences, such progeny may not, in fact, be identical to the parent cell, but are still included within the scope of the term as used herein. Typical host cells are eukaryotic host cells, such as mammalian host cells. Exemplary eukaryotic host cells include yeast and mammalian cells, for example vertebrate cells such as a mouse, rat, monkey, or human cell line, for example HKB11 cells, PER.C6 cells, HEK cells or CHO cells.

Immunoglobulin: The term “immunoglobulin” (Ig) refers to a class of structurally related glycoproteins consisting of two pairs of polypeptide chains, one pair of light (L) chains and one pair of heavy (H) chains, which may all four be inter-connected by disulfide bonds. The structure of immunoglobulins has been well characterized. See for instance Fundamental Immunology Ch. 7 (Paul, W., ed., 2nd ed. Raven Press, N. Y. (1989)). Each heavy chain typically comprises a heavy chain variable region (abbreviated herein as VH or VH) and a heavy chain constant region (CH or CH). The heavy chain constant region typically comprises three domains, CH1, CH2, and CH3. The CH1 and CH2 domains are linked by a hinge. The Fc portion comprises at least the CH2 and CH3 domains.

Typically, the numbering of amino acid residues of immunoglobulins is according to IMGT, Sequences of Proteins of Immunological Interest, 5th Ed. Public Health Service, National Institutes of Health, Bethesda, MD. (1991), or by the EU numbering system of Kabat (also known as “EU numbering” or “EU index”), e.g., as in Kabat et al. Sequences of Proteins of Immunological interest. 5th ed. US Department of Health and Human Services, NIH publication No. 91-3242 (1991).

Linker: The term “linker” as used herein refers to a connecting peptide between two moieties. For example, a linker can connect a spike protein ABD to an Fc domain.

Multispecific: The term “multispecific” as used herein refers to antigen-binding molecules comprising two or more ABDs. For instance, ABDs in a multispecific molecule can bind to two or more different portions of the same target antigen (or, in the case of a viral protein, different variants of the same target antigen such as spike protein) or each ABD can bind to a different target antigen.

Multivalent: The term “multivalent” as used herein refers to an antigen-binding molecule comprising two or more ABDs, on one, two or more polypeptide chains.

Neutralizing, Blocking: A “neutralizing” or “blocking” spike protein ABD refers to an ABD, whose binding to spike protein inhibits an activity of the spike protein to any detectable degree, e.g., inhibits the ability of spike protein to bind to a receptor such as ACE2, to be cleaved by a protease such as TMPRSS2, or to mediate viral entry into a host cell or viral reproduction in a host cell.

Operably linked: The term “operably linked” refers to a functional relationship between two or more peptide or polypeptide domains or nucleic acid (e.g., DNA) segments. In the context of a fusion protein or other polypeptide, the term “operably linked” means that two or more amino acid segments are linked so as to produce a functional polypeptide. For example, in the context of a multivalent anti-spike protein binding molecule of the disclosure, separate components (e.g., a spike protein ABD and an Fc domain, a J chain and an IgG Fc domain, etc.) can be operably linked directly or through peptide linker sequences. In the context of a nucleic acid encoding a fusion protein, such as a multivalent anti-spike protein binding molecule of the disclosure, “operably linked” means that the two nucleic acids are joined such that the amino acid sequences encoded by the two nucleic acids remain in-frame.

Polypeptide, Peptide and Protein: The terms “polypeptide”, “peptide” and “protein” are used interchangeably herein to refer to a polymer of amino acid residues.

Recognize: The term “recognize” as used herein refers to an antibody or antibody fragment (e.g., a spike protein ABD) that finds and interacts (e.g., binds) with its epitope.

Single Chain Fab or scFab: The term “single chain Fab” or “scFab” as used herein refers to a polypeptide chain comprising the VH, CH1, VL and CL domains of antibody, where these domains are present in a single polypeptide chain.

Single Chain Fv or scFv: The term “single-chain Fv” or “scFv” as used herein refers to ABDs comprising the VH and VL domains of an antibody, wherein these domains are present in a single polypeptide chain. Preferably, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds. (1994), Springer-Verlag, New York, pp. 269-315. The VH and VL and be arranged in the N- to C-terminal order VH-VL or VL-VH, typically separated by a linker.

Subject: The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

Treat, Treatment, Treating: As used herein, the terms “treat”, “treatment” and “treating” refer to the reduction or amelioration of the progression, severity and/or duration of a disease or condition and/or the amelioration of one or more symptoms (preferably, one or more discernible symptoms) of a disease or condition resulting from the administration of one or more multivalent anti-spike protein binding molecules of the disclosure.

In some embodiments, the disease or condition is caused by a coronavirus infection, for example SARS-CoV or SARS-CoV-2, for example COVID-19. In some embodiments, the disease or condition is any other ailment associated with SARS-CoV or SARS-CoV-2 infection, or similar infections. With reference to these diseases and conditions, the terms “treat”, “treatment” and “treating” refer to the reduction or amelioration of the progression, severity and/or duration of the disease or the amelioration of one or more symptoms (preferably, one or more discernible symptoms) of a disease resulting from the administration of one or more multivalent anti-spike protein binding molecules of the disclosure. In specific embodiments, the terms “treat”, “treatment” and “treating” refer to the amelioration of at least one measurable physical parameter of COVID-19, such as blood oxygen saturation levels, not necessarily discernible by the patient. In other embodiments the terms “treat”, “treatment” and “treating” refer to the inhibition of the progression of COVID-19, either physically by, e.g., stabilization of a discernible symptom, physiologically by, e.g., stabilization of a physical parameter, or both. In other embodiments the terms “treat”, “treatment” and “treating” refer to the reduction or elimination of infection.

### 6.2. Spike Protein Antigen-Binding Domain

The present disclosure relates to multivalent anti-spike protein binding molecules of the disclosure comprising a plurality of spike protein antigen binding domains (ABDs).

In some embodiments, a multivalent anti-spike protein binding molecule of the disclosure comprises ten or twelve spike protein ABDs.

In some embodiments, the multivalent anti-spike protein binding molecules of the disclosure are monospecific, e.g., bind the same epitope on spike protein. In some of these embodiments, the anti-spike protein ABDs are identical.

In other embodiments, the multivalent anti-spike protein binding molecules of the disclosure are multispecific, e.g., bind to different epitopes. In some embodiments, the multispecific anti-spike protein binding molecules bind to different epitopes on the same spike protein. In other embodiments, multispecific anti-spike protein binding molecules bind to different epitopes on different spike protein variants. The different epitopes can correspond to sequence variants of the same region in a spike protein or in different regions altogether.

In further embodiments, a plurality or all of the ABDs in the multivalent anti-spike protein binding molecules of the disclosure bind to the receptor binding domain (RBD) of a spike protein and/or are capable of blocking or neutralizing spike protein, e.g., inhibit the ability of spike protein to bind to a receptor such as ACE2, to be cleaved by a protease such as TMPRSS2, or to mediate viral entry into a host cell or viral reproduction in a host cell.

Suitable spike protein ABD formats are described in Section 6.3. The spike protein ABD can be, for example, an antibody or an antigen-binding portion of an antibody, e.g., a Fab, as described in Section 6.3.1 or an scFv, as described in Section 6.3.2.

In some embodiments, a spike protein ABD competes with exemplary antibody or an antibody having the sequence set forth in Table 1 below for binding to spike protein and/or comprises binding portions of an exemplary antibody or an antibody having an antibody sequence set forth in Table 1. In some aspects, the spike protein ABD competes with an antibody set forth in Table 1 for binding to a spike protein. In further aspects, the spike protein ABD comprises CDRs having CDR sequences of an antibody set forth in Table 1. In some embodiments, the spike protein ABD comprises all 6 CDR sequences of the antibody set forth in Table 1. In other embodiments, the spike protein ABD comprises at least the heavy chain CDR sequences (CDR-H1, CDR-H2, CDR-H3 and the light chain CDR sequences of a universal light chain. In further aspects, a spike protein ABD comprises a VH comprising the amino acid sequence of the VH of an antibody set forth in Table 1. In some embodiments, the spike protein ABD further comprises a VL comprising the amino acid sequence of the VL of the antibody set forth in Table 1. In other embodiments, the spike protein ABD further comprises a universal light chain VL sequence.

TABLE 1
Spike Protein Antibodies and Antigen-Binding Domains

| | | Blocking/Non- |
|---|---|---|
| | | blocking ACE2 |
| Target/Epitope | Antibody Name and/or Binding Sequences | binding |
| SARS-CoV-2 | P5-22 | Blocking |
| Spike protein | VH: SEQ ID NO: 2 of PCT Publication No: WO | |
| | 2022/052968 A1 | |
| | VL: SEQ ID NO: 4 of PCT Publication No: WO | |
| | 2022/052968 A1 | |
| SARS-CoV-2 | P14-44 | Blocking |
| Spike protein | VH: SEQ ID NO: 6 of PCT Publication No: WO | |
| | 2022/052968 A1 | |
| | VL: SEQ ID NO: 8 of PCT Publication No: WO | |
| | 2022/052968 A1 | |
| SARS-CoV-2 | P15-16 | Blocking |
| Spike protein | VH: SEQ ID NO: 10 of PCT Publication No: WO | |
| | 2022/052968 A1 | |
| | VL: SEQ ID NO: 12 of PCT Publication No: WO | |
| | 2022/052968 A1 | |
| SARS-CoV-2 | P10-20 | Blocking |
| Spike protein | VH: SEQ ID NO: 14 of PCT Publication No: WO | |
| | 2022/052968 A1 | |
| | VL: SEQ ID NO: 16 of PCT Publication No: WO | |
| | 2022/052968 A1 | |
| SARS-CoV-2 | P14-37 | Blocking |
| Spike protein | VH: SEQ ID NO: 18 of PCT Publication No: WO | |
| | 2022/052968 A1 | |
| | VL: SEQ ID NO: 20 of PCT Publication No: WO | |
| | 2022/052968 A1 | |
| SARS-CoV-2 | P23-29 | Blocking |
| Spike protein | VH: SEQ ID NO: 22 of PCT Publication No: WO | |
| | 2022/052968 A1 | |
| | VL: SEQ ID NO: 24 of PCT Publication No: WO | |
| | 2022/052968 A1 | |
| SARS-CoV-2 | P3-11 | Blocking |
| Spike protein | VH: SEQ ID NO: 26 of PCT Publication No: WO | |
| | 2022/052968 A1 | |
| | VL: SEQ ID NO: 28 of PCT Publication No: WO | |
| | 2022/052968 A1 | |
| aa 319-532 of S- | P16-A3 | Blocking |
| protein RBD | VH: SEQ ID NO: 17 of PCT Publication No: WO | |
| domain | 2021/196268 A1 | |
| | VL: SEQ ID NO: 18 of PCT Publication No: WO | |
| | 2021/196268 A1 | |
| aa 319-532 of S- | P17-A11 | Blocking |
| protein RBD | VH: SEQ ID NO: 19 of PCT Publication No: WO | |
| domain | 2021/196268 A1 | |
| | VL: SEQ ID NO: 20 of PCT Publication No: WO | |
| | 2021/196268 A1 | |
| aa 319-532 of S- | R15-F7 | Blocking |
| protein RBD | VH: SEQ ID NO: 1 of PCT Publication No: WO | |
| domain | 2022/104918 A1 | |
| | VL: SEQ ID NO: 2 of PCT Publication No: WO | |
| | 2022/104918 A1 | |
| S-protein RBD | DH1041 | Blocking |
| domain | VH: SEQ ID NO: 1100 of PCT Publication No: WO | |
| | 2022/060906 A1 | |
| | VL: SEQ ID NO: 1102 of PCT Publication No: WO | |
| | 2022/060906 A1 | |
| aa 481-491 of S- | DH1042 | Blocking |
| protein RBD | VH: SEQ ID NO: 1104 of PCT Publication No: WO | |
| domain | 2022/060906 A1 | |
| | VL: SEQ ID NO: 1106 of PCT Publication No: WO | |
| | 2022/060906 A1 | |
| S-protein RBD | DH1043 | Blocking |
| domain | VH: SEQ ID NO: 1108 of PCT Publication No: WO | |
| | 2022/060906 A1 | |
| | VL: SEQ ID NO: 1110 of PCT Publication No: WO | |
| | 2022/060906 A1 | |
| S-protein RBD | DH1044 | Non-blocking |
| domain | VH: SEQ ID NO: 1112 of PCT Publication No: WO | |
| | 2022/060906 A1 | |
| | VL: SEQ ID NO: 1114 of PCT Publication No: WO | |
| | 2022/060906 A1 | |
| S-protein RBD | DH1221 | Blocking |
| domain | VH: SEQ ID NO: 1214 of PCT Publication No: WO | |
| | 2022/060906 A1 | |
| | VL: SEQ ID NO: 1226 of PCT Publication No: WO | |
| | 2022/060906 A1 | |
| S-protein RBD | DH1222 | Blocking |
| domain | VH: SEQ ID NO: 1216 of PCT Publication No: WO | |
| | 2022/060906 A1 | |
| | VL: SEQ ID NO: 1228 of PCT Publication No: WO | |
| | 2022/060906 A1 | |
| S-protein RBD | DH1223 | Blocking |
| domain | VH: SEQ ID NO: 1218 of PCT Publication No: WO | |
| | 2022/060906 A1 | |
| | VL: SEQ ID NO: 1230 of PCT Publication No: WO | |
| | 2022/060906 A1 | |
| S-protein RBD | DH1224 | Blocking |
| domain | VH: SEQ ID NO: 1220 of PCT Publication No: WO | |
| | 2022/060906 A1 | |
| | VL: SEQ ID NO: 1232 of PCT Publication No: WO | |
| | 2022/060906 A1 | |
| S-protein RBD | DH1225 | Blocking |
| domain | VH: SEQ ID NO: 1222 of PCT Publication No: WO | |
| | 2022/060906 A1 | |
| | VL: SEQ ID NO: 1234 of PCT Publication No: WO | |
| | 2022/060906 A1 | |
| S-protein RBD | DH1226 | Blocking |
| domain | VH: SEQ ID NO: 1224 of PCT Publication No: WO | |
| | 2022/060906 A1 | |
| | VL: SEQ ID NO: 1236 of PCT Publication No: WO | |
| | 2022/060906 A1 | |
| S-protein RBD | mAb S1D2 | Blocking |
| domain | VH: SEQ ID NO: 6 of PCT Publication No: WO | |
| SEQ ID NO: 5 of | 2022/032139 A1 | |
| PCT WO | VL: SEQ ID NO: 7 of PCT Publication No: WO | |
| 2022/032139 A1 | 2022/032139 A1 | |
| S-protein RBD | mAb S1D7270 | Blocking |
| domain | VH: SEQ ID NO: 28 of PCT Publication No: WO | |
| SEQ ID NO: 5 of | 2022/032139 A1 | |
| PCT WO | VL: SEQ ID NO: 29 of PCT Publication No: WO | |
| 2022/032139 A1 | 2022/032139 A1 | |
| aa 331-524 of S- | 413-2 | |
| protein RBD | VH: SEQ ID NO: 15 of PCT Publication No: WO | |
| domain | 2021/248279 A1 | |
| SEQ ID NO: 61 | VL: SEQ ID NO: 43 of PCT Publication No: WO | |
| | 2021/248279 A1 | |
| aa 331-524 of S- | 413-3 | |
| protein RBD | VH: SEQ ID NO: 16 of PCT Publication No: WO | |
| domain | 2021/248279 A1 | |
| SEQ ID NO: 61 | VL: SEQ ID NO: 44 of PCT Publication No: WO | |
| | 2021/248279 A1 | |
| aa 331-524 of S- | 505-1 | |
| protein RBD | VH: SEQ ID NO: 17 of PCT Publication No: WO | |
| domain | 2021/248279 A1 | |
| SEQ ID NO: 61 | VL: SEQ ID NO: 45 of PCT Publication No: WO | |
| | 2021/248279 A1 | |
| aa 331-524 of S- | 505-3 | |
| protein RBD | VH: SEQ ID NO: 18 of PCT Publication No: WO | |
| domain | 2021/248279 A1 | |
| SEQ ID NO: 61 | VL: SEQ ID NO: 46 of PCT Publication No: WO | |
| | 2021/248279 A1 | |
| aa 331-524 of S- | 505-5 | |
| protein RBD | VH: SEQ ID NO: 19 of PCT Publication No: WO | |
| domain | 2021/248279 A1 | |
| SEQ ID NO: 61 | VL: SEQ ID NO: 47 of PCT Publication No: WO | |
| | 2021/248279 A1 | |
| aa 331-524 of S- | 515-5 | |
| protein RBD | VH: SEQ ID NO: 20 of PCT Publication No: WO | |
| domain | 2021/248279 A1 | |
| SEQ ID NO: 61 | VL: SEQ ID NO: 48 of PCT Publication No: WO | |
| | 2021/248279 A1 | |
| aa 331-524 of S- | 553-13 | |
| protein RBD | VH: SEQ ID NO: 21 of PCT Publication No: WO | |
| domain | 2021/248279 A1 | |
| SEQ ID NO: 61 | VL: SEQ ID NO: 49 of PCT Publication No: WO | |
| | 2021/248279 A1 | |
| aa 331-524 of S- | 553-15 | |
| protein RBD | VH: SEQ ID NO: 22 of PCT Publication No: WO | |
| domain | 2021/248279 A1 | |
| SEQ ID NO: 61 | VL: SEQ ID NO: 50 of PCT Publication No: WO | |
| | 2021/248279 A1 | |
| aa 331-524 of S- | 553-17 | |
| protein RBD | VH: SEQ ID NO: 23 of PCT Publication No: WO | |
| domain | 2021/248279 A1 | |
| SEQ ID NO: 61 | VL: SEQ ID NO: 51 of PCT Publication No: WO | |
| | 2021/248279 A1 | |
| aa 331-524 of S- | 553-18 | |
| protein RBD | VH: SEQ ID NO: 24 of PCT Publication No: WO | |
| domain | 2021/248279 A1 | |
| SEQ ID NO: 61 | VL: SEQ ID NO: 52 of PCT Publication No: WO | |
| | 2021/248279 A1 | |
| aa 331-524 of S- | 553-20 | |
| protein RBD | VH: SEQ ID NO: 25 of PCT Publication No: WO | |
| domain | 2021/248279 A1 | |
| SEQ ID NO: 61 | VL: SEQ ID NO: 53 of PCT Publication No: WO | |
| | 2021/248279 A1 | |
| aa 331-524 of S- | 553-27 | |
| protein RBD | VH: SEQ ID NO: 26 of PCT Publication No: WO | |
| domain | 2021/248279 A1 | |
| SEQ ID NO: 61 | VL: SEQ ID NO: 54 of PCT Publication No: WO | |
| | 2021/248279 A1 | |
| aa 331-524 of S- | 553-60 | |
| protein RBD | VH: SEQ ID NO: 27 of PCT Publication No: WO | |
| domain | 2021/248279 A1 | |
| SEQ ID NO: 61 | VL: SEQ ID NO: 55 of PCT Publication No: WO | |
| | 2021/248279 A1 | |
| aa 331-524 of S- | 553-63 | |
| protein RBD | VH: SEQ ID NO: 28 of PCT Publication No: WO | |
| domain | 2021/248279 A1 | |
| SEQ ID NO: 61 | VL: SEQ ID NO: 56 of PCT Publication No: WO | |
| | 2021/248279 A1 | |
| SARS-CoV-2 | N3-1 | |
| Spike protein | VH: SEQ ID NO: 1 of PCT Publication No: WO | |
| RBD | 2022/192661 A1 | |
| | VL: SEQ ID NO: 2 of PCT Publication No: WO | |
| | 2022/192661 A1 | |
| SARS-CoV-2 | N3-3 | |
| Spike protein | VH: SEQ ID NO: 1 of PCT Publication No: WO | |
| RBD | 2022/192661 A1 | |
| | VL: SEQ ID NO: 15 of PCT Publication No: WO | |
| | 2022/192661 A1 | |
| SARS-CoV-2 | 1D4 | |
| Spike protein | VH: SEQ ID NO: 18 of PCT Publication No: WO | |
| RBD | 2022/192661 A1 | |
| | VL: SEQ ID NO: 19 of PCT Publication No: WO | |
| | 2022/192661 A1 | |
| SARS-CoV-2 | N3-7 | |
| Spike protein | VH: SEQ ID NO: 1 of PCT Publication No: WO | |
| RBD | 2022/192661 A1 | |
| | VL: SEQ ID NO: 10 of PCT Publication No: WO | |
| | 2022/192661 A1 | |
| SARS-CoV-2 | N3-63A | |
| Spike protein | VH: SEQ ID NO: 1 of PCT Publication No: WO | |
| RBD | 2022/192661 A1 | |
| | VL: SEQ ID NO: 21 of PCT Publication No: WO | |
| | 2022/192661 A1 | |
| SARS-CoV-2 | N3-63B | |
| Spike protein | VH: SEQ ID NO: 1 of PCT Publication No: WO | |
| RBD | 2022/192661 A1 | |
| | VL: SEQ ID NO: 27 of PCT Publication No: WO | |
| | 2022/192661 A1 | |
| SARS-CoV-2 | N3-63C | |
| Spike protein | VH: SEQ ID NO: 1 of PCT Publication No: WO | |
| RBD | 2022/192661 A1 | |
| | VL: SEQ ID NO: 37 of PCT Publication No: WO | |
| | 2022/192661 A1 | |
| SARS-CoV-2 | 8B5 | |
| Spike protein | VH: SEQ ID NO: 22 of PCT Publication No: WO | |
| RBD | 2022/192661 A1 | |
| | VL: SEQ ID NO: 23 of PCT Publication No: WO | |
| | 2022/192661 A1 | |
| SARS-CoV-2 | 4A5 | |
| Spike protein | VH: SEQ ID NO: 24 of PCT Publication No: WO | |
| RBD | 2022/192661 A1 | |
| | VL: SEQ ID NO: 23 of PCT Publication No: WO | |
| | 2022/192661 A1 | |
| SARS-CoV-2 | 1D1 | |
| Spike protein | VH: SEQ ID NO: 18 of PCT Publication No: WO | |
| RBD | 2022/192661 A1 | |
| | VL: SEQ ID NO: 2 of PCT Publication No: WO | |
| | 2022/192661 A1 | |
| SARS-CoV-2 | 1D9 | |
| Spike protein | VH: SEQ ID NO: 18 of PCT Publication No: WO | |
| RBD | 2022/192661 A1 | |
| | VL: SEQ ID NO: 26 of PCT Publication No: WO | |
| | 2022/192661 A1 | |
| SARS-CoV-2 | 4A7 | |
| Spike protein | VH: SEQ ID NO: 24 of PCT Publication No: WO | |
| RBD | 2022/192661 A1 | |
| | VL: SEQ ID NO: 10 of PCT Publication No: WO | |
| | 2022/192661 A1 | |
| SARS-CoV-2 | 1D5 | |
| Spike protein | VH: SEQ ID NO: 18 of PCT Publication No: WO | |
| RBD | 2022/192661 A1 | |
| | VL: SEQ ID NO: 23 of PCT Publication No: WO | |
| | 2022/192661 A1 | |

In some embodiments, the spike protein ABDs comprise an amino acid sequence or are encoded by a nucleotide sequence set forth in Table 2 below. In particular aspects, the spike protein ABD comprises both heavy and light chain CDRs of an antibody set forth in Table 2 below. In other embodiments, the spike protein ABD comprises at least the heavy chain CDR sequences and the light chain CDR sequences of a universal light chain. In further aspects, a spike protein ABD comprises a VH having the amino acid sequence of the VH of an antibody set forth in Table 2 and a VL having the amino acid sequence of the VL of the same antibody as set forth in Table 2. In other aspects, a spike protein ABD comprises a VH having the amino acid sequence of the VH of an antibody set forth in Table 2 and a universal light chain VL sequence.

TABLE 2

| Antibody | Component | |
|---|---|---|
| Designation | Part | Sequence |
| mAb10933 | Amino Acids | |
| | VH | QVQLVESGGGLVKPGGSLRLSCAASGFTFSDYYMSWIRQAP |
| | | GKGLEWVSYITYSGSTIYYADSVKGRFTISRDNAKSSLYLQ |
| | | MNSLRAEDTAVYYCARDRGTTMVPFDYWGQGTLVTVSS |
| | | (SEQ ID NO: 10) |
| | CDR-H1 | GFTFSDYY (SEQ ID NO: 11) |
| | CDR-H2 | ITYSGSTI (SEQ ID NO: 12) |
| | CDR-H3 | ARDRGTTMVPFDY (SEQ ID NO: 13) |
| | VL | DIQMTQSPSSLSASVGDRVTITCQASQDITNYLNWYQQKPG |
| | | KAPKLLIYAASNLETGVPSRFSGSGSGTDFTFTISGLQPED |
| | | IATYYCQQYDNLPLTFGGGTKVEIK (SEQ ID NO: 14) |
| | CDR-L1 | QDITNY (SEQ ID NO: 15) |
| | CDR-L2 | AAS |
| | CDR-L3 | QQYDNLPLT (SEQ ID NO: 16) |
| | HC | QVQLVESGGGLVKPGGSLRLSCAASGFTFSDYYMSWIRQAP |
| | | GKGLEWVSYITYSGSTIYYADSVKGRFTISRDNAKSSLYLQ |
| | | MNSLRAEDTAVYYCARDRGTTMVPFDYWGQGTLVTVSSAST |
| | | KGPSVFPLAPSSKSTSGGTAALGCLVKDYFPEPVTVSWNSG |
| | | ALTSGVHTFPAVLQSSGLYSLSSVVTVPSSSLGTQTYICNV |
| | | NHKPSNTKVDKKVEPKSCDKTHTCPPCPAPELLGGPSVFLF |
| | | PPKPKDTLMISRTPEVTCVVVDVSHEDPEVKFNWYVDGVEV |
| | | HNAKTKPREEQYNSTYRVVSVLTVLHQDWLNGKEYKCKVSN |
| | | KALPAPIEKTISKAKGQPREPQVYTLPPSRDELTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPGK |
| | | (SEQ ID NO: 17) |
| | LC | DIQMTQSPSSLSASVGDRVTITCQASQDITNYLNWYQQKPG |
| | | KAPKLLIYAASNLETGVPSRESGSGSGTDFTFTISGLQPED |
| | | IATYYCQQYDNLPLTFGGGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC (SEQ ID NO: 18) |
| | VH | CAGGTGCAGCTGGTGGAGTCTGGGGGAGGCTTGGTCAAGCC |
| | | TGGAGGGTCCCTGAGACTCTCCTGTGCAGCCTCTGGATTCA |
| | | CCTTCAGTGACTACTACATGAGCTGGATCCGCCAGGCTCCA |
| | | GGGAAGGGGCTGGAGTGGGTTTCATACATTACTTATAGTGG |
| | | TAGTACCATATACTACGCAGACTCTGTGAAGGGCCGATTCA |
| | | CCATCTCCAGGGACAACGCCAAGAGCTCACTGTATCTGCAA |
| | | ATGAACAGCCTGAGAGCCGAGGACACGGCCGTGTATTACTG |
| | | TGCGAGAGATCGCGGTACAACTATGGTCCCCTTTGACTACT |
| | | GGGGCCAGGGAACCCTGGTCACCGTCTCCTCA (SEQ ID |
| | | NO: 19) |
| | CDR-H1 | GGATTCACCTTCAGTGACTACTAC (SEQ ID NO: 20) |
| | CDR-H2 | ATTACTTATAGTGGTAGTACCATA (SEQ ID NO: 21) |
| | CDR-H3 | GCGAGAGATCGCGGTACAACTATGGTCCCCTTTGACTAC |
| | | (SEQ ID NO: 22) |
| | VL | GACATCCAGATGACCCAGTCTCCATCCTCCCTGTCTGCATC |
| | | TGTAGGAGACAGAGTCACCATCACTTGCCAGGCGAGTCAGG |
| | | ACATTACCAACTATTTAAATTGGTATCAGCAGAAACCAGGG |
| | | AAAGCCCCTAAGCTCCTGATCTACGCTGCATCCAATTTGGA |
| | | AACAGGGGTCCCATCAAGGTTCAGTGGAAGTGGATCTGGGA |
| | | CAGATTTTACTTTCACCATCAGCGGCCTGCAGCCTGAAGAT |
| | | ATTGCAACATATTACTGTCAACAGTATGATAATCTCCCTCT |
| | | CACTTTCGGCGGAGGGACCAAGGTGGAGATCAAA (SEQ |
| | | ID NO: 23) |
| | CDR-L1 | CAGGACATTACCAACTAT (SEQ ID NO: 24) |
| | CDR-L2 | GCTGCATCC |
| | CDR-L3 | CAACAGTATGATAATCTCCCTCTCACT (SEQ ID |
| | | NO: 25) |
| | HC | CAGGTGCAGCTGGTGGAGTCTGGGGGAGGCTTGGTCAAGCC |
| | | TGGAGGGTCCCTGAGACTCTCCTGTGCAGCCTCTGGATTCA |
| | | CCTTCAGTGACTACTACATGAGCTGGATCCGCCAGGCTCCA |
| | | GGGAAGGGGCTGGAGTGGGTTTCATACATTACTTATAGTGG |
| | | TAGTACCATATACTACGCAGACTCTGTGAAGGGCCGATTCA |
| | | CCATCTCCAGGGACAACGCCAAGAGCTCACTGTATCTGCAA |
| | | ATGAACAGCCTGAGAGCCGAGGACACGGCCGTGTATTACTG |
| | | TGCGAGAGATCGCGGTACAACTATGGTCCCCTTTGACTACT |
| | | GGGGCCAGGGAACCCTGGTCACCGTCTCCTCAGCCTCCACC |
| | | AAGGGCCCATCGGTCTTCCCCCTGGCACCCTCCTCCAAGAG |
| | | CACCTCTGGGGGCACAGCGGCCCTGGGCTGCCTGGTCAAGG |
| | | ACTACTTCCCCGAACCGGTGACGGTGTCGTGGAACTCAGGC |
| | | GCCCTGACCAGCGGCGTGCACACCTTCCCGGCTGTCCTACA |
| | | GTCCTCAGGACTCTACTCCCTCAGCAGCGTGGTGACCGTGC |
| | | CCTCCAGCAGCTTGGGCACCCAGACCTACATCTGCAACGTG |
| | | AATCACAAGCCCAGCAACACCAAGGTGGACAAGAAAGTTGA |
| | | GCCCAAATCTTGTGACAAAACTCACACATGCCCACCGTGCC |
| | | CAGCACCTGAACTCCTGGGGGGACCGTCAGTCTTCCTCTTC |
| | | CCCCCAAAACCCAAGGACACCCTCATGATCTCCCGGACCCC |
| | | TGAGGTCACATGCGTGGTGGTGGACGTGAGCCACGAAGACC |
| | | CTGAGGTCAAGTTCAACTGGTACGTGGACGGCGTGGAGGTG |
| | | CATAATGCCAAGACAAAGCCGCGGGAGGAGCAGTACAACAG |
| | | CACGTACCGTGTGGTCAGCGTCCTCACCGTCCTGCACCAGG |
| | | ACTGGCTGAATGGCAAGGAGTACAAGTGCAAGGTCTCCAAC |
| | | AAAGCCCTCCCAGCCCCCATCGAGAAAACCATCTCC |
| | | AAAGCCAAAGGGCAGCCCCGAGAACCACAGGTGTACACCCT |
| | | GCCCCCATCCCGGGATGAGCTGACCAAGAACCAGGTCAGCC |
| | | TGACCTGCCTGGTCAAAGGCTTCTATCCCAGCGACATCGCC |
| | | GTGGAGTGGGAGAGCAATGGGCAGCCGGAGAACAACTACAA |
| | | GACCACGCCTCCCGTGCTGGACTCCGACGGCTCCTTCTTCC |
| | | TCTACAGCAAGCTCACCGTGGACAAGAGCAGGTGGCAGCAG |
| | | GGGAACGTCTTCTCATGCTCCGTGATGCATGAGGCTCTGCA |
| | | CAACCACTACACGCAGAAGTCCCTCTCCCTGTCTCCGGGTA |
| | | AATGA (SEQ ID NO: 26) |
| | LC | GACATCCAGATGACCCAGTCTCCATCCTCCCTGTCTGCATC |
| | | TGTAGGAGACAGAGTCACCATCACTTGCCAGGCGAGTCAGG |
| | | ACATTACCAACTATTTAAATTGGTATCAGCAGAAACCAGGG |
| | | AAAGCCCCTAAGCTCCTGATCTACGCTGCATCCAATTTGGA |
| | | AACAGGGGTCCCATCAAGGTTCAGTGGAAGTGGATCTGGGA |
| | | CAGATTTTACTTTCACCATCAGCGGCCTGCAGCCTGAAGAT |
| | | ATTGCAACATATTACTGTCAACAGTATGATAATCTCCCTCT |
| | | CACTTTCGGCGGAGGGACCAAGGTGGAGATCAAACGAACTG |
| | | TGGCTGCACCATCTGTCTTCATCTTCCCGCCATCTGATGAG |
| | | CAGTTGAAATCTGGAACTGCCTCTGTTGTGTGCCTGCTGAA |
| | | TAACTTCTATCCCAGAGAGGCCAAAGTACAGTGGAAGGTGG |
| | | ATAACGCCCTCCAATCGGGTAACTCCCAGGAGAGTGTCACA |
| | | GAGCAGGACAGCAAGGACAGCACCTACAGCCTCAGCAGCAC |
| | | CCTGACGCTGAGCAAAGCAGACTACGAGAAACACAAAGTCT |
| | | ACGCCTGCGAAGTCACCCATCAGGGCCTGAGCTCGCCCGTC |
| | | ACAAAGAGCTTCAACAGGGGAGAGTGTTAG (SEQ ID |
| | | NO: 27) |
| mAb10934 | VH | EVQLVESGGGLVKPGGSLRLSCAASGITFSNAWMSWVRQAP |
| | | GKGLEWVGRIKSKTDGGTTDYAAPVKGRFTISRDDSKNTLY |
| | | LQMNSLKTEDTAVYYCTTARWDWYFDLWGRGTLVTVSS |
| | | (SEQ ID NO: 28) |
| | CDR-H1 | GITFSNAW (SEQ ID NO: 29) |
| | CDR-H2 | IKSKTDGGTT (SEQ ID NO: 30) |
| | CDR-H3 | TTARWDWYFDL (SEQ ID NO: 31) |
| | VL | DIQMTQSPSSLSASVGDRVTITCQASQDIWNYINWYQQKPG |
| | | KAPKLLIYDASNLKTGVPSRFSGSGSGTDFTFTISSLQPED |
| | | IATYYCQQHDDLPPTFGQGTKVEIK (SEQ ID NO: 32) |
| | CDR-L1 | QDIWNY (SEQ ID NO: 33) |
| | CDR-L2 | DAS |
| | CDR-L3 | QQHDDLPPT (SEQ ID NO: 34) |
| | HC | EVQLVESGGGLVKPGGSLRLSCAASGITESNAWMSWVRQAP |
| | | GKGLEWVGRIKSKTDGGTTDYAAPVKGRFTISRDDSKNTLY |
| | | LQMNSLKTEDTAVYYCTTARWDWYFDLWGRGTLVTVSSAST |
| | | KGPSVFPLAPSSKSTSGGTAALGCLVKDYFPEPVTVSWNSG |
| | | ALTSGVHTFPAVLQSSGLYSLSSVVTVPSSSLGTQTYICNV |
| | | NHKPSNTKVDKKVEPKSCDKTHTCPPCPAPELLGGPSVFLF |
| | | PPKPKDTLMISRTPEVTCVVVDVSHEDPEVKFNWYVDGVEV |
| | | HNAKTKPREEQYNSTYRVVSVLTVLHQDWLNGKEYKCKVSN |
| | | KALPAPIEKTISKAKGQPREPQVYTLPPSRDELTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPGK |
| | | (SEQ ID NO: 35) |
| | LC | DIQMTQSPSSLSASVGDRVTITCQASQDIWNYINWYQQKPG |
| | | KAPKLLIYDASNLKTGVPSRFSGSGSGTDFTFTISSLQPED |
| | | IATYYCQQHDDLPPTFGQGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC (SEQ ID NO: 36) |
| | VH | GAGGTGCAGCTGGTGGAGTCTGGGGGAGGCTTGGTAAAGCC |
| | | TGGGGGGTCCCTTAGACTCTCCTGTGCAGCCTCTGGAATCA |
| | | CTTTCAGTAACGCCTGGATGAGTTGGGTCCGCCAGGCTCCA |
| | | GGGAAGGGGCTGGAGTGGGTTGGCCGTATTAAAAGCAAAAC |
| | | TGATGGTGGGACAACAGACTACGCCGCACCCGTGAAAGGCA |
| | | GATTCACCATCTCAAGAGATGATTCAAAAAACACGCTGTAT |
| | | CTACAAATGAACAGCCTGAAAACCGAGGACACAGCCGTGTA |
| | | TTACTGTACCACAGCGAGGTGGGACTGGTACTTCGATCTCT |
| | | GGGGCCGTGGCACCCTGGTCACTGTCTCCTCA (SEQ ID |
| | | NO: 37) |
| | CDR-H1 | GGAATCACTTTCAGTAACGCCTGG (SEQ ID NO: 38) |
| | CDR-H2 | ATTAAAAGCAAAACTGATGGTGGGACAACA (SEQ ID |
| | | NO: 39) |
| | CDR-H3 | ACCACAGCGAGGTGGGACTGGTACTTCGATCTC (SEQ ID |
| | | NO: 40) |
| | VL | GACATCCAGATGACCCAGTCTCCATCCTCCCTGTCTGCATC |
| | | TGTAGGAGACAGAGTCACCATCACTTGCCAGGCGAGTCAGG |
| | | ACATTTGGAATTATATAAATTGGTATCAGCAGAAACCAGGG |
| | | AAGGCCCCTAAGCTCCTGATCTACGATGCATCCAATTTGAA |
| | | AACAGGGGTCCCATCAAGGTTCAGTGGAAGTGGATCTGGGA |
| | | CAGATTTTACTTTCACCATCAGCAGCCTGCAGCCTGAAGAT |
| | | ATTGCAACATATTACTGTCAACAGCATGATGATCTCCCTCC |
| | | GACCTTCGGCCAAGGGACCAAGGTGGAAATCAAA (SEQ |
| | | ID NO: 41) |
| | CDR-L1 | CAGGACATTTGGAATTAT (SEQ ID NO: 42) |
| | CDR-L2 | GATGCATCC |
| | CDR-L3 | CAACAGCATGATGATCTCCCTCCGACC (SEQ ID |
| | | NO: 43) |
| | HC | GAGGTGCAGCTGGTGGAGTCTGGGGGAGGCTTGGTAAAGCC |
| | | TGGGGGGTCCCTTAGACTCTCCTGTGCAGCCTCTGGAATCA |
| | | CTTTCAGTAACGCCTGGATGAGTTGGGTCCGCCAGGCTCCA |
| | | GGGAAGGGGCTGGAGTGGGTTGGCCGTATTAAAAGCAAAAC |
| | | TGATGGTGGGACAACAGACTACGCCGCACCCGTGAAAGGCA |
| | | GATTCACCATCTCAAGAGATGATTCAAAAAACACGCTGTAT |
| | | CTACAAATGAACAGCCTGAAAACCGAGGACACAGCCGTGTA |
| | | TTACTGTACCACAGCGAGGTGGGACTGGTACTTCGATCTCT |
| | | GGGGCCGTGGCACCCTGGTCACTGTCTCCTCAGCCTCCACC |
| | | AAGGGCCCATCGGTCTTCCCCCTGGCACCCTCCTCCAAGAG |
| | | CACCTCTGGGGGCACAGCGGCCCTGGGCTGCCTGGTCAAGG |
| | | ACTACTTCCCCGAACCGGTGACGGTGTCGTGGAACTCAGGC |
| | | GCCCTGACCAGCGGCGTGCACACCTTCCCGGCTGTCCTACA |
| | | GTCCTCAGGACTCTACTCCCTCAGCAGCGTGGTGACCGTGC |
| | | CCTCCAGCAGCTTGGGCACCCAGACCTACATCTGCAACGTG |
| | | AATCACAAGCCCAGCAACACCAAGGTGGACAAGAAAGTTGA |
| | LC | GCCCAAATCTTGTGACAAAACTCACACATGCCCACCGTGCC |
| | | CAGCACCTGAACTCCTGGGGGGACCGTCAGTCTTCCTCTTC |
| | | CCCCCAAAACCCAAGGACACCCTCATGATCTCCCGGACCCC |
| | | TGAGGTCACATGCGTGGTGGTGGACGTGAGCCACGAAGACC |
| | | CTGAGGTCAAGTTCAACTGGTACGTGGACGGCGTGGAGGTG |
| | | CATAATGCCAAGACAAAGCCGCGGGAGGAGCAGTACAACAG |
| | | CACGTACCGTGTGGTCAGCGTCCTCACCGTCCTGCACCAGG |
| | | ACTGGCTGAATGGCAAGGAGTACAAGTGCAAGGTCTCCAAC |
| | | AAAGCCCTCCCAGCCCCCATCGAGAAAACCATCTCC |
| | | AAAGCCAAAGGGCAGCCCCGAGAACCACAGGTGTACACCCT |
| | | GCCCCCATCCCGGGATGAGCTGACCAAGAACCAGGTCAGCC |
| | | TGACCTGCCTGGTCAAAGGCTTCTATCCCAGCGACATCGCC |
| | | GTGGAGTGGGAGAGCAATGGGCAGCCGGAGAACAACTACAA |
| | | GACCACGCCTCCCGTGCTGGACTCCGACGGCTCCTTCTTCC |
| | | TCTACAGCAAGCTCACCGTGGACAAGAGCAGGTGGCAGCAG |
| | | GGGAACGTCTTCTCATGCTCCGTGATGCATGAGGCTCTGCA |
| | | CAACCACTACACGCAGAAGTCCCTCTCCCTGTCTCCGGGTA |
| | | AATGA (SEQ ID NO: 44) |
| | | GACATCCAGATGACCCAGTCTCCATCCTCCCTGTCTGCATC |
| | | TGTAGGAGACAGAGTCACCATCACTTGCCAGGCGAGTCAGG |
| | | ACATTTGGAATTATATAAATTGGTATCAGCAGAAACCAGGG |
| | | AAGGCCCCTAAGCTCCTGATCTACGATGCATCCAATTTGAA |
| | | AACAGGGGTCCCATCAAGGTTCAGTGGAAGTGGATCTGGGA |
| | | CAGATTTTACTTTCACCATCAGCAGCCTGCAGCCTGAAGAT |
| | | ATTGCAACATATTACTGTCAACAGCATGATGATCTCCCTCC |
| | | GACCTTCGGCCAAGGGACCAAGGTGGAAATCAAACGAACTG |
| | | TGGCTGCACCATCTGTCTTCATCTTCCCGCCATCTGATGAG |
| | | CAGTTGAAATCTGGAACTGCCTCTGTTGTGTGCCTGCTGAA |
| | | TAACTTCTATCCCAGAGAGGCCAAAGTACAGTGGAAGGTGG |
| | | ATAACGCCCTCCAATCGGGTAACTCCCAGGAGAGTGTCACA |
| | | GAGCAGGACAGCAAGGACAGCACCTACAGCCTCAGCAGCAC |
| | | CCTGACGCTGAGCAAAGCAGACTACGAGAAACACAAAGTCT |
| | | ACGCCTGCGAAGTCACCCATCAGGGCCTGAGCTCGCCCGTC |
| | | ACAAAGAGCTTCAACAGGGGAGAGTGTTAG (SEQ ID |
| | | NO: 45) |
| mAb10987 | VH | QVQLVESGGGVVQPGRSLRLSCAASGFTFSNYAMYWVRQAP |
| | | GKGLEWVAVISYDGSNKYYADSVKGRFTISRDNSKNTLYLQ |
| | | MNSLRTEDTAVYYCASGSDYGDYLLVYWGQGTLVTVSS |
| | | (SEQ ID NO: 46) |
| | CDR-H1 | GFTFSNYA (SEQ ID NO: 47) |
| | CDR-H2 | ISYDGSNK (SEQ ID NO: 48) |
| | CDR-H3 | ASGSDYGDYLLVY (SEQ ID NO: 49) |
| | VL | QSALTQPASVSGSPGQSITISCTGTSSDVGGYNYVSWYQQH |
| | | PGKAPKLMIYDVSKRPSGVSNRFSGSKSGNTASLTISGLQS |
| | | EDEADYYCNSLTSISTWVEGGGTKLTVL (SEQ ID |
| | | NO: 50) |
| | CDR-L1 | SSDVGGYNY (SEQ ID NO: 51) |
| | CDR-L2 | DVS |
| | CDR-L3 | NSLTSISTWV (SEQ ID NO: 52) |
| | HC | QVQLVESGGGVVQPGRSLRLSCAASGFTFSNYAMYWVRQAP |
| | | GKGLEWVAVISYDGSNKYYADSVKGRFTISRDNSKNTLYLQ |
| | | MNSLRTEDTAVYYCASGSDYGDYLLVYWGQGTLVTVSSAST |
| | | KGPSVFPLAPSSKSTSGGTAALGCLVKDYFPEPVTVSWNSG |
| | | ALTSGVHTFPAVLQSSGLYSLSSVVTVPSSSLGTQTYICNV |
| | | NHKPSNTKVDKKVEPKSCDKTHTCPPCPAPELLGGPSVFLF |
| | | PPKPKDTLMISRTPEVTCVVVDVSHEDPEVKFNWYVDGVEV |
| | | HNAKTKPREEQYNSTYRVVSVLTVLHQDWLNGKEYKCKVSN |
| | | KALPAPIEKTISKAKGQPREPQVYTLPPSRDELTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPGK |
| | | (SEQ ID NO: 53) |
| | LC | QSALTQPASVSGSPGQSITISCTGTSSDVGGYNYVSWYQQH |
| | | PGKAPKLMIYDVSKRPSGVSNRFSGSKSGNTASLTISGLQS |
| | | EDEADYYCNSLTSISTWVFGGGTKLTVLGQPKAAPSVTLEP |
| | | PSSEELQANKATLVCLISDFYPGAVTVAWKADSSPVKAGVE |
| | | TTTPSKQSNNKYAASSYLSLTPEQWKSHRSYSCQVTHEGST |
| | | VEKTVAPTECS (SEQ ID NO: 55) |
| | VH | CAGGTGCAGCTGGTGGAGTCTGGGGGAGGCGTGGTCCAGCC |
| | | TGGGAGGTCCCTGAGACTCTCCTGTGCAGCCTCTGGATTCA |
| | | CCTTCAGTAACTATGCTATGTACTGGGTCCGCCAGGCTCCA |
| | | GGCAAGGGGCTGGAGTGGGTGGCAGTTATATCATATGATGG |
| | | AAGTAATAAATACTATGCAGACTCCGTGAAGGGCCGATTCA |
| | | CCATCTCCAGAGACAATTCCAAGAACACGCTGTATCTGCAA |
| | | ATGAACAGCCTGAGAACTGAGGACACGGCTGTGTATTACTG |
| | | TGCGAGTGGCTCCGACTACGGTGACTACTTATTGGTTTACT |
| | | GGGGCCAGGGAACCCTGGTCACCGTCTCCTCA (SEQ ID |
| | | NO: 55) |
| | CDR-H1 | GGATTCACCTTCAGTAACTATGCT (SEQ ID NO: 56) |
| | CDR-H2 | ATATCATATGATGGAAGTAATAAA (SEQ ID NO: 57) |
| | CDR-H3 | GCGAGTGGCTCCGACTACGGTGACTACTTATTGGTTTAC |
| | | (SEQ ID NO: 58) |
| | VL | CAGTCTGCCCTGACTCAGCCTGCCTCCGTGTCTGGGTCTCC |
| | | TGGACAGTCGATCACCATCTCCTGCACTGGAACCAGCAGTG |
| | | ACGTTGGTGGTTATAACTATGTCTCCTGGTACCAACAACAC |
| | | CCAGGCAAAGCCCCCAAACTCATGATTTATGATGTCAGTAA |
| | | GCGGCCCTCAGGGGTTTCTAATCGCTTCTCTGGCTCCAAGT |
| | | CTGGCAACACGGCCTCCCTGACCATCTCTGGGCTCCAGTCT |
| | | GAGGACGAGGCTGATTATTACTGCAACTCTTTGACAAGCAT |
| | | CAGCACTTGGGTGTTCGGCGGAGGGACCAAGCTGACCGTCC |
| | | TA (SEQ ID NO: 59) |
| | CDR-L1 | AGCAGTGACGTTGGTGGTTATAACTAT (SEQ ID |
| | | NO: 60) |
| | CDR-L2 | GATGTCAGT |
| | CDR-L3 | AACTCTTTGACAAGCATCAGCACTTGGGTG (SEQ ID |
| | | NO: 61) |
| | HC | CAGGTGCAGCTGGTGGAGTCTGGGGGAGGCGTGGTCCAGCC |
| | | TGGGAGGTCCCTGAGACTCTCCTGTGCAGCCTCTGGATTCA |
| | | CCTTCAGTAACTATGCTATGTACTGGGTCCGCCAGGCTCCA |
| | | GGCAAGGGGCTGGAGTGGGTGGCAGTTATATCATATGATGG |
| | | AAGTAATAAATACTATGCAGACTCCGTGAAGGGCCGATTCA |
| | | CCATCTCCAGAGACAATTCCAAGAACACGCTGTATCTGCAA |
| | | ATGAACAGCCTGAGAACTGAGGACACGGCTGTGTATTACTG |
| | | TGCGAGTGGCTCCGACTACGGTGACTACTTATTGGTTTACT |
| | | GGGGCCAGGGAACCCTGGTCACCGTCTCCTCAGCCTCCACC |
| | | AAGGGCCCATCGGTCTTCCCCCTGGCACCCTCCTCCAAGAG |
| | | CACCTCTGGGGGCACAGCGGCCCTGGGCTGCCTGGTCAAGG |
| | | ACTACTTCCCCGAACCGGTGACGGTGTCGTGGAACTCAGGC |
| | | GCCCTGACCAGCGGCGTGCACACCTTCCCGGCTGTCCTACA |
| | | GTCCTCAGGACTCTACTCCCTCAGCAGCGTGGTGACCGTGC |
| | | CCTCCAGCAGCTTGGGCACCCAGACCTACATCTGCAACGTG |
| | | AATCACAAGCCCAGCAACACCAAGGTGGACAAGAAAGTTGA |
| | | GCCCAAATCTTGTGACAAAACTCACACATGCCCACCGTGCC |
| | | CAGCACCTGAACTCCTGGGGGGACCGTCAGTCTTCCTCTTC |
| | | CCCCCAAAACCCAAGGACACCCTCATGATCTCCCGGACCCC |
| | | TGAGGTCACATGCGTGGTGGTGGACGTGAGCCACGAAGACC |
| | | CTGAGGTCAAGTTCAACTGGTACGTGGACGGCGTGGAGGTG |
| | | CATAATGCCAAGACAAAGCCGCGGGAGGAGCAGTACAACAG |
| | | CACGTACCGTGTGGTCAGCGTCCTCACCGTCCTGCACCAGG |
| | | ACTGGCTGAATGGCAAGGAGTACAAGTGCAAGGTCTCCAAC |
| | | AAAGCCCTCCCAGCCCCCATCGAGAAAACCATCTCC |
| | | AAAGCCAAAGGGCAGCCCCGAGAACCACAGGTGTACACCCT |
| | | GCCCCCATCCCGGGATGAGCTGACCAAGAACCAGGTCAGCC |
| | | TGACCTGCCTGGTCAAAGGCTTCTATCCCAGCGACATCGCC |
| | | GTGGAGTGGGAGAGCAATGGGCAGCCGGAGAACAACTACAA |
| | | GACCACGCCTCCCGTGCTGGACTCCGACGGCTCCTTCTTCC |
| | | TCTACAGCAAGCTCACCGTGGACAAGAGCAGGTGGCAGCAG |
| | | GGGAACGTCTTCTCATGCTCCGTGATGCATGAGGCTCTGCA |
| | | CAACCACTACACGCAGAAGTCCCTCTCCCTGTCTCCGGGTA |
| | | AATGA (SEQ ID NO: 62) |
| | LC | CAGTCTGCCCTGACTCAGCCTGCCTCCGTGTCTGGGTCTCC |
| | | TGGACAGTCGATCACCATCTCCTGCACTGGAACCAGCAGTG |
| | | ACGTTGGTGGTTATAACTATGTCTCCTGGTACCAACAACAC |
| | | CCAGGCAAAGCCCCCAAACTCATGATTTATGATGTCAGTAA |
| | | GCGGCCCTCAGGGGTTTCTAATCGCTTCTCTGGCTCCAAGT |
| | | CTGGCAACACGGCCTCCCTGACCATCTCTGGGCTCCAGTCT |
| | | GAGGACGAGGCTGATTATTACTGCAACTCTTTGACAAGCAT |
| | | CAGCACTTGGGTGTTCGGCGGAGGGACCAAGCTGACCGTCC |
| | | TAGGCCAGCCCAAGGCCGCCCCCTCCGTGACCCTGTTCCCC |
| | | CCCTCCTCCGAGGAGCTGCAGGCCAACAAGGCCACCCTGGT |
| | | GTGCCTGATCTCCGACTTCTACCCCGGCGCCGTGACCGTGG |
| | | CCTGGAAGGCCGACTCCTCCCCCGTGAAGGCCGGCGTGGAG |
| | | ACCACCACCCCCTCCAAGCAGTCCAACAACAAGTACGCCGC |
| | | CTCCTCCTACCTGTCCCTGACCCCCGAGCAGTGGAAGTCCC |
| | | ACCGGTCCTACTCCTGCCAGGTGACCCACGAGGGCTCCACC |
| | | GTGGAGAAGACCGTGGCCCCCACCGAGTGCTCCTGA (SEQ |
| | | ID NO: 63) |
| mAb10989 | VH | QVQLVQSGAEVKKPGASVKVSCKASGYIFTGYYMHWVRQAP |
| | | GQGLEWMGWINPNSGGANYAQKFQGRVTLTRDTSITTVYME |
| | | LSRLREDDTAVYYCARGSRYDWNQNNWEDPWGQGTLVTVSS |
| | | (SEQ ID NO: 64) |
| | CDR-H1 | GYIFTGYY (SEQ ID NO: 65) |
| | CDR-H2 | INPNSGGA (SEQ ID NO: 66) |
| | CDR-H3 | ARGSRYDWNQNNWFDP (SEQ ID NO: 67) |
| | VL | QSALTQPASVSGSPGQSITISCTGTSSDVGTYNYVSWYQQH |
| | | PGKAPKLMIFDVSNRPSGVSDRFSGSKSGNTASLTISGLQA |
| | | EDEADYYCSSFTTSSTVVFGGGTKLTVL (SEQ ID |
| | | NO: 68) |
| | CDR-L1 | SSDVGTYNY (SEQ ID NO: 69) |
| | CDR-L2 | DVS |
| | CDR-L3 | SSFTTSSTVV (SEQ ID NO: 70) |
| | HC | QVQLVQSGAEVKKPGASVKVSCKASGYIFTGYYMHWVRQAP |
| | | GQGLEWMGWINPNSGGANYAQKFQGRVTLTRDTSITTVYME |
| | | LSRLREDDTAVYYCARGSRYDWNQNNWEDPWGQGTLVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYI |
| | | CNVNHKPSNTKVDKKVEPKSCDKTHTCPPCPAPELLGGPSV |
| | | FLFPPKPKDTLMISRTPEVTCVVVDVSHEDPEVKFNWYVDG |
| | | VEVHNAKTKPREEQYNSTYRVVSVLTVLHQDWLNGKEYKCK |
| | | VSNKALPAPIEKTISKAKGQPREPQVYTLPPSRDELTKNQV |
| | | SLTCLVKGFYPSDIAVEWESNGQPENNYKTTPPVLDSDGSF |
| | | FLYSKLTVDKSRWQQGNVFSCSVMHEALHNHYTQKSLSLSP |
| | | GK (SEQ ID NO: 71) |
| | LC | QSALTQPASVSGSPGQSITISCTGTSSDVGTYNYVSWYQQH |
| | | PGKAPKLMIFDVSNRPSGVSDRFSGSKSGNTASLTISGLQA |
| | | EDEADYYCSSFTTSSTVVFGGGTKLTVLGQPKAAPSVTLEP |
| | | PSSEELQANKATLVCLISDFYPGAVTVAWKADSSPVKAGVE |
| | | TTTPSKQSNNKYAASSYLSLTPEQWKSHRSYSCQVTHEGST |
| | | VEKTVAPTECS (SEQ ID NO: 72) |
| | VH | CAGGTGCAGCTGGTGCAGTCTGGGGCTGAGGTGAAGAAGCC |
| | | TGGGGCCTCAGTGAAGGTCTCCTGCAAGGCTTCTGGATACA |
| | | TCTTCACCGGCTACTATATGCACTGGGTGCGACAGGCCCCT |
| | | GGACAGGGGCTTGAGTGGATGGGATGGATCAACCCTAACAG |
| | | TGGTGGCGCAAACTATGCACAGAAGTTTCAGGGCAGGGTCA |
| | | CCCTGACCAGGGACACGTCCATCACCACAGTCTACATGGAA |
| | | CTGAGCAGGCTGAGATTTGACGACACGGCCGTGTATTACTG |
| | | TGCGAGAGGATCCCGGTATGACTGGAACCAGAACAACTGGT |
| | | TCGACCCCTGGGGCCAGGGAACCCTGGTCACCGTCTCCTCA |
| | | (SEQ ID NO: 73) |
| | CDR-H1 | GGATACATCTTCACCGGCTACTAT (SEQ ID NO: 74) |
| | CDR-H2 | ATCAACCCTAACAGTGGTGGCGCA (SEQ ID NO: 75) |
| | CDR-H3 | GCGAGAGGATCCCGGTATGACTGGAACCAGAACAACTGGTT |
| | | CGACCCC (SEQ ID NO: 76) |
| | VL | CAGTCTGCCCTGACTCAGCCTGCCTCCGTGTCTGGGTCTCC |
| | | TGGACAGTCGATCACCATCTCCTGCACTGGAACCAGCAGTG |
| | | ACGTTGGTACTTATAACTATGTCTCCTGGTACCAACAACAC |
| | | CCAGGCAAAGCCCCCAAACTCATGATTTTTGATGTCAGTAA |
| | | TCGGCCCTCAGGGGTTTCTGATCGCTTCTCTGGCTCCAAGT |
| | | CTGGCAACACGGCCTCCCTGACCATCTCTGGGCTCCAGGCT |
| | | GAGGACGAGGCTGATTATTACTGCAGCTCATTTACAACCAG |
| | | CAGCACTGTGGTTTTCGGCGGAGGGACCAAGCTGACCGTCC |
| | | TA (SEQ ID NO: 77) |
| | CDR-L1 | AGCAGTGACGTTGGTACTTATAACTAT (SEQ ID |
| | | NO: 78) |
| | CDR-L2 | GATGTCAGT |
| | CDR-L3 | AGCTCATTTACAACCAGCAGCACTGTGGTT (SEQ ID |
| | | NO: 79) |
| | HC | CAGGTGCAGCTGGTGCAGTCTGGGGCTGAGGTGAAGAAGCC |
| | | TGGGGCCTCAGTGAAGGTCTCCTGCAAGGCTTCTGGATACA |
| | | TCTTCACCGGCTACTATATGCACTGGGTGCGACAGGCCCCT |
| | | GGACAGGGGCTTGAGTGGATGGGATGGATCAACCCTAACAG |
| | | TGGTGGCGCAAACTATGCACAGAAGTTTCAGGGCAGGGTCA |
| | | CCCTGACCAGGGACACGTCCATCACCACAGTCTACATGGAA |
| | | CTGAGCAGGCTGAGATTTGACGACACGGCCGTGTATTACTG |
| | | TGCGAGAGGATCCCGGTATGACTGGAACCAGAACAACTGGT |
| | | TCGACCCCTGGGGCCAGGGAACCCTGGTCACCGTCTCCTCA |
| | | GCCTCCACCAAGGGCCCATCGGTCTTCCCCCTGGCACCCTC |
| | | CTCCAAGAGCACCTCTGGGGGCACAGCGGCCCTGGGCTGCC |
| | | TGGTCAAGGACTACTTCCCCGAACCGGTGACGGTGTCGTGG |
| | | AACTCAGGCGCCCTGACCAGCGGCGTGCACACCTTCCCGGC |
| | | TGTCCTACAGTCCTCAGGACTCTACTCCCTCAGCAGCGTGG |
| | | TGACCGTGCCCTCCAGCAGCTTGGGCACCCAGACCTACATC |
| | | TGCAACGTGAATCACAAGCCCAGCAACACCAAGGTGGACAA |
| | | GAAAGTTGAGCCCAAATCTTGTGACAAAACTCACACATGCC |
| | | CACCGTGCCCAGCACCTGAACTCCTGGGGGGACCGTCAGTC |
| | | TTCCTCTTCCCCCCAAAACCCAAGGACACCCTCATGATCTC |
| | | CCGGACCCCTGAGGTCACATGCGTGGTGGTGGACGTGAGCC |
| | | ACGAAGACCCTGAGGTCAAGTTCAACTGGTACGTGGACGGC |
| | | GTGGAGGTGCATAATGCCAAGACAAAGCCGCGGGAGGAGCA |
| | | GTACAACAGCACGTACCGTGTGGTCAGCGTCCTCACCGTCC |
| | | TGCACCAGGACTGGCTGAATGGCAAGGAGTACAAGTGCAAG |
| | | GTCTCCAACAAAGCCCTCCCAGCCCCCATCGAGAAA |
| | | ACCATCTCCAAAGCCAAAGGGCAGCCCCGAGAACCACAGGT |
| | | GTACACCCTGCCCCCATCCCGGGATGAGCTGACCAAGAACC |
| | | AGGTCAGCCTGACCTGCCTGGTCAAAGGCTTCTATCCCAGC |
| | | GACATCGCCGTGGAGTGGGAGAGCAATGGGCAGCCGGAGAA |
| | | CAACTACAAGACCACGCCTCCCGTGCTGGACTCCGACGGCT |
| | | CCTTCTTCCTCTACAGCAAGCTCACCGTGGACAAGAGCAGG |
| | | TGGCAGCAGGGGAACGTCTTCTCATGCTCCGTGATGCATGA |
| | | GGCTCTGCACAACCACTACACGCAGAAGTCCCTCTCCCTGT |
| | | CTCCGGGTAAATGA (SEQ ID NO: 80) |
| | LC | CAGTCTGCCCTGACTCAGCCTGCCTCCGTGTCTGGGTCTCC |
| | | TGGACAGTCGATCACCATCTCCTGCACTGGAACCAGCAGTG |
| | | ACGTTGGTACTTATAACTATGTCTCCTGGTACCAACAACAC |
| | | CCAGGCAAAGCCCCCAAACTCATGATTTTTGATGTCAGTAA |
| | | TCGGCCCTCAGGGGTTTCTGATCGCTTCTCTGGCTCCAAGT |
| | | CTGGCAACACGGCCTCCCTGACCATCTCTGGGCTCCAGGCT |
| | | GAGGACGAGGCTGATTATTACTGCAGCTCATTTACAACCAG |
| | | CAGCACTGTGGTTTTCGGCGGAGGGACCAAGCTGACCGTCC |
| | | TAGGCCAGCCCAAGGCCGCCCCCTCCGTGACCCTGTTCCCC |
| | | CCCTCCTCCGAGGAGCTGCAGGCCAACAAGGCCACCCTGGT |
| | | GTGCCTGATCTCCGACTTCTACCCCGGCGCCGTGACCGTGG |
| | | CCTGGAAGGCCGACTCCTCCCCCGTGAAGGCCGGCGTGGAG |
| | | ACCACCACCCCCTCCAAGCAGTCCAACAACAAGTACGCCGC |
| | | CTCCTCCTACCTGTCCCTGACCCCCGAGCAGTGGAAGTCCC |
| | | ACCGGTCCTACTCCTGCCAGGTGACCCACGAGGGCTCCACC |
| | | GTGGAGAAGACCGTGGCCCCCACCGAGTGCTCCTGA (SEQ |
| | | ID NO: 81) |

In further embodiments, the spike protein ABDs comprise an amino acid sequence set forth in Table 3 below. In particular aspects, the spike protein ABD comprises both heavy and light chain CDRs of an antibody set forth in Table 3 below. In other embodiments, the spike protein ABD comprises at least the heavy chain CDR sequences and the light chain CDR sequences of a universal light chain. In further aspects, a spike protein ABD comprises a VH having the amino acid sequence of the VH of an antibody set forth in Table 3 and a VL having the amino acid sequence of the VL of the same antibody as set forth in Table 3. In other aspects, a spike protein ABD comprises a VH having the amino acid sequence of the VH of an antibody set forth in Table 3 and a universal light chain VL sequence. The initial sequence identifiers for Table 3 are in relation to the sequence listing in WO 2021/045836A1, which sequence identifiers are incorporated by reference herein, while sequence identifiers presented parenthetically are those of the disclosure.

TABLE 3
SEQ ID NO in WO2021/045836A1 (SEQ ID NO of the disclosure)

| Antibody | | | | | | | | |
|---|---|---|---|---|---|---|---|---|
| Designation | VH | CDR-H1 | CDR-H2 | CDR-H3 | VL | CDR-L1 | CDR-L2 | CDR-L3 |
| mAb10913 | 2 | 4 | 6 | 8 | 10 | 12 | 14 | 16 |
| | (88) | (89) | (90) | (91) | (92) | (93) | (94) | (95) |
| mAb10915 | 22 | 24 | 26 | 28 | 30 | 32 | 34 | 36 |
| | (96) | (97) | (98) | (99) | (100) | (101) | (102) | (103) |
| mAb10916 | 2 | 4 | 6 | 8 | 10 | 12 | 14 | 16 |
| | (88) | (89) | (90) | (91) | (92) | (93) | (94) | (95) |
| mAb10917 | 44 | 46 | 26 | 49 | 51 | 53 | 55 | 57 |
| | (104) | (105) | (98) | (106) | (107) | (108) | (109) | (110) |
| mAb10918 | 22 | 24 | 26 | 28 | 30 | 32 | 34 | 36 |
| | (96) | (97) | (98) | (99) | (100) | (101) | (102) | (103) |
| mAb10920 | 65 | 67 | 69 | 71 | 73 | 75 | 55 | 77 |
| | (111) | (112) | (113) | (114) | (115) | (116) | (109) | (117) |
| mAb10921 | 83 | 85 | 26 | 87 | 89 | 91 | 55 | 93 |
| | (118) | (119) | (98) | (120) | (121) | (122) | (109) | (123) |
| mAb10922 | 99 | 101 | 103 | 105 | 107 | 109 | 111 | 113 |
| | (124) | (125) | (126) | (127) | (128) | (129) | (130) | (131) |
| mAb10923 | 119 | 121 | 123 | 125 | 127 | 129 | 55 | 131 |
| | (132) | (133) | (134) | (135) | (136) | (137) | (109) | (138) |
| mAb10924 | 137 | 139 | 141 | 143 | 145 | 147 | 149 | 151 |
| | (139) | (140) | (141) | (142) | (143) | (144) | (145) | (146) |
| mAb10925 | 65 | 67 | 69 | 71 | 73 | 75 | 55 | 77 |
| | (111) | (112) | (113) | (114) | (115) | (116) | (109) | (117) |
| mAb10926 | 83 | 85 | 26 | 87 | 89 | 91 | 55 | 93 |
| | (118) | (119) | (98) | (120) | (121) | (122) | (109) | (123) |
| mAb10927 | 99 | 101 | 103 | 105 | 107 | 109 | 111 | 113 |
| | (124) | (125) | (126) | (127) | (128) | (129) | (130) | (131) |
| mAb10928 | 119 | 121 | 123 | 125 | 127 | 129 | 55 | 131 |
| | (132) | (133) | (134) | (135) | (136) | (137) | (109) | (138) |
| mAb10929 | 137 | 139 | 141 | 143 | 145 | 147 | 149 | 151 |
| | (139) | (140) | (141) | (142) | (143) | (144) | (145) | (146) |
| mAb10930 | 167 | 169 | 171 | 173 | 175 | 129 | 55 | 177 |
| | (147) | (148) | (149) | (150) | (151) | (137) | (109) | (152) |
| mAb10931 | 167 | 169 | 171 | 173 | 175 | 129 | 55 | 177 |
| | (147) | (148) | (149) | (150) | (151) | (137) | (109) | (152) |
| mAb10932 | 185 | 187 | 26 | 189 | 191 | 75 | 194 | 196 |
| | (153) | (154) | (98) | (155) | (156) | (116) | (157) | (158) |
| mAb10933 | 202 | 204 | 206 | 208 | 210 | 212 | 55 | 214 |
| | (10) | (11) | (12) | (13) | (14) | (15) | (109) | (16) |
| mAb10934 | 220 | 222 | 224 | 226 | 228 | 230 | 194 | 232 |
| | (28) | (29) | (30) | (31) | (32) | (33) | (157) | (34) |
| mAb10935 | 238 | 24 | 26 | 240 | 242 | 244 | 194 | 246 |
| | (173) | (97) | (98) | (174) | (175) | (176) | (157) | (177) |
| mAb10936 | 252 | 254 | 256 | 258 | 260 | 129 | 55 | 262 |
| | (178) | (179) | (180) | (181) | (182) | (137) | (109) | (183) |
| mAb10937 | 268 | 270 | 272 | 274 | 276 | 129 | 55 | 278 |
| | (184) | (185) | (186) | (187) | (188) | (137) | (109) | (189) |
| mAb10940 | 284 | 169 | 286 | 288 | 290 | 292 | 294 | 296 |
| | (190) | (148) | (191) | (192) | (193) | (194) | (195) | (196) |
| mAb10938 | 302 | 24 | 26 | 304 | 306 | 308 | 194 | 310 |
| | (197) | (97) | (98) | (198) | (199) | (200) | (157) | (201) |
| mAb10939 | 316 | 187 | 319 | 321 | 323 | 325 | 55 | 327 |
| | (202) | (154) | (203) | (204) | (205) | (206) | (109) | (207) |
| mAb10941 | 333 | 85 | 26 | 336 | 338 | 340 | 294 | 296 |
| | (208) | (119) | (98) | (209) | (210) | (211) | (195) | (196) |
| mAb10942 | 185 | 187 | 26 | 189 | 191 | 75 | 194 | 196 |
| | (153) | (154) | (98) | (155) | (156) | (116) | (157) | (158) |
| mAb10943 | 202 | 204 | 206 | 208 | 210 | 212 | 55 | 214 |
| | (10) | (11) | (12) | (13) | (14) | (15) | (109) | (16) |
| mAb10944 | 220 | 222 | 224 | 226 | 228 | 230 | 194 | 232 |
| | (28) | (29) | (30) | (31) | (32) | (33) | (157) | (34) |
| mAb10945 | 238 | 24 | 26 | 240 | 242 | 244 | 194 | 246 |
| | (173) | (97) | (98) | (174) | (175) | (176) | (157) | (177) |
| mAb10946 | 252 | 254 | 256 | 258 | 260 | 129 | 55 | 262 |
| | (178) | (179) | (180) | (181) | (182) | (137) | (109) | (183) |
| mAb10947 | 268 | 270 | 272 | 274 | 276 | 129 | 55 | 278 |
| | (184) | (185) | (186) | (187) | (188) | (137) | (109) | (189) |
| mAb10948 | 302 | 24 | 26 | 304 | 306 | 308 | 194 | 310 |
| | (197) | (97) | (98) | (198) | (199) | (200) | (157) | (201) |
| mAb10949 | 316 | 187 | 319 | 321 | 323 | 325 | 55 | 327 |
| | (202) | (154) | (203) | (204) | (205) | (206) | (109) | (207) |
| mAb10951 | 333 | 85 | 26 | 336 | 338 | 340 | 294 | 296 |
| | (208) | (119) | (98) | (209) | (210) | (211) | (195) | (196) |
| mAb10950 | 284 | 169 | 286 | 288 | 290 | 292 | 294 | 296 |
| | (190) | (148) | (191) | (192) | (193) | (194) | (195) | (196) |
| mAb10954 | 366 | 85 | 26 | 370 | 372 | 244 | 194 | 375 |
| | (212) | (119) | (98) | (213) | (214) | (176) | (157) | (215) |
| mAb10955 | 381 | 383 | 26 | 385 | 387 | 389 | 194 | 310 |
| | (216) | (217) | (98) | (218) | (219) | (220) | (157) | (201) |
| mAb10956 | 396 | 187 | 26 | 399 | 401 | 389 | 194 | 403 |
| | (221) | (154) | (98) | (222) | (223) | (220) | (157) | (224) |
| mAb10957 | 409 | 411 | 26 | 414 | 416 | 53 | 55 | 418 |
| | (225) | (226) | (98) | (227) | (228) | (108) | (109) | (229) |
| mAb10958 | 366 | 85 | 26 | 370 | 372 | 244 | 194 | 375 |
| | (212) | (119) | (98) | (213) | (214) | (176) | (157) | (215) |
| mAb10959 | 381 | 383 | 26 | 385 | 387 | 389 | 194 | 310 |
| | (216) | (217) | (98) | (218) | (219) | (220) | (157) | (201) |
| mAb10960 | 396 | 187 | 26 | 399 | 401 | 389 | 194 | 403 |
| | (221) | (154) | (98) | (222) | (223) | (220) | (157) | (224) |
| mAb10961 | 409 | 411 | 26 | 414 | 416 | 53 | 55 | 418 |
| | (225) | (226) | (98) | (227) | (228) | (108) | (109) | (229) |
| mAb10964 | 432 | 434 | 436 | 438 | 440 | 442 | 55 | 445 |
| | (230) | (231) | (232) | (233) | (234) | (235) | (109) | (236) |
| mAb10965 | 451 | 453 | 26 | 455 | 457 | 459 | 34 | 462 |
| | (237) | (238) | (98) | (239) | (240) | (241) | (102) | (242) |
| mAb10966 | 468 | 187 | 26 | 470 | 472 | 389 | 194 | 474 |
| | (243) | (154) | (98) | (244) | (245) | (220) | (157) | (246) |
| mAb10967 | 480 | 24 | 483 | 485 | 487 | 389 | 194 | 489 |
| | (247) | (97) | (248) | (249) | (250) | (220) | (157) | (251) |
| mAb10969 | 495 | 497 | 499 | 501 | 503 | 389 | 194 | 214 |
| | (252) | (253) | (254) | (255) | (256) | (220) | (157) | (165) |
| mAb10970 | 510 | 24 | 26 | 512 | 514 | 516 | 194 | 518 |
| | (257) | (97) | (98) | (258) | (259) | (260) | (157) | (261) |
| mAb10971 | 524 | 411 | 26 | 528 | 530 | 532 | 55 | 534 |
| | (262) | (226) | (98) | (263) | (264) | (265) | (109) | (266) |
| mAb10973 | 432 | 434 | 436 | 438 | 440 | 442 | 55 | 445 |
| | (230) | (231) | (232) | (233) | (234) | (235) | (109) | (236) |
| mAb10974 | 451 | 453 | 26 | 455 | 457 | 459 | 34 | 462 |
| | (237) | (238) | (98) | (239) | (240) | (241) | (102) | (242) |
| mAb10975 | 468 | 187 | 26 | 470 | 472 | 389 | 194 | 474 |
| | (243) | (154) | (98) | (244) | (245) | (220) | (157) | (246) |
| mAb10976 | 480 | 24 | 483 | 485 | 487 | 389 | 194 | 489 |
| | (247) | (97) | (248) | (249) | (250) | (220) | (157) | (251) |
| mAb10977 | 548 | 550 | 552 | 554 | 556 | 558 | 294 | 560 |
| | (267) | (268) | (269) | (270) | (271) | (272) | (195) | (273) |
| mAb10978 | 495 | 497 | 499 | 501 | 503 | 389 | 194 | 214 |
| | (252) | (253) | (254) | (255) | (256) | (220) | (157) | (165) |
| mAb10979 | 510 | 24 | 26 | 512 | 514 | 516 | 194 | 518 |
| | (257) | (97) | (98) | (258) | (259) | (260) | (157) | (261) |
| mAb10980 | 524 | 411 | 26 | 528 | 530 | 532 | 55 | 534 |
| | (262) | (226) | (98) | (263) | (264) | (265) | (109) | (266) |
| mAb10981 | 548 | 550 | 552 | 554 | 556 | 558 | 294 | 560 |
| | (267) | (268) | (269) | (270) | (271) | (272) | (195) | (273) |
| mAb10982 | 574 | 187 | 576 | 578 | 580 | 582 | 584 | 586 |
| | (274) | (154) | (275) | (276) | (277) | (278) | (279) | (280) |
| mAb10983 | 574 | 187 | 576 | 578 | 580 | 582 | 584 | 586 |
| | (274) | (154) | (275) | (276) | (277) | (278) | (279) | (280) |
| mAb10984 | 594 | 596 | 26 | 598 | 600 | 12 | 14 | 602 |
| | (281) | (282) | (98) | (283) | (284) | (93) | (94) | (285) |
| mAb10985 | 608 | 169 | 610 | 612 | 614 | 616 | 584 | 618 |
| | (286) | (148) | (287) | (288) | (289) | (290) | (279) | (291) |
| mAb10986 | 624 | 626 | 26 | 628 | 630 | 582 | 632 | 634 |
| | (292) | (293) | (98) | (294) | (295) | (278) | (296) | (297) |
| mAb10987 | 640 | 642 | 499 | 644 | 646 | 648 | 650 | 652 |
| | (46) | (47) | (48) | (49) | (50) | (51) | (303) | (52) |
| mAb10988 | 658 | 660 | 662 | 664 | 666 | 668 | 670 | 672 |
| | (305) | (306) | (307) | (308) | (309) | (310) | (311) | (312) |
| mAb10989 | 678 | 680 | 682 | 684 | 686 | 688 | 650 | 690 |
| | (64) | (65) | (66) | (67) | (68) | (69) | (303) | (70) |
| mAb10990 | 594 | 596 | 26 | 598 | 600 | 12 | 14 | 602 |
| | (281) | (282) | (98) | (283) | (284) | (93) | (94) | (285) |
| mAb10991 | 608 | 169 | 610 | 612 | 614 | 616 | 584 | 618 |
| | (286) | (148) | (287) | (288) | (289) | (290) | (279) | (291) |
| mAb10992 | 624 | 626 | 26 | 628 | 630 | 582 | 632 | 634 |
| | (292) | (293) | (98) | (294) | (295) | (278) | (296) | (297) |
| mAb10993 | 640 | 642 | 499 | 644 | 646 | 648 | 650 | 652 |
| | (46) | (47) | (48) | (49) | (50) | (51) | (303) | (52) |
| mAb10994 | 658 | 660 | 662 | 664 | 666 | 668 | 670 | 672 |
| | (305) | (306) | (307) | (308) | (309) | (310) | (311) | (312) |
| mAb10995 | 678 | 680 | 682 | 684 | 686 | 688 | 650 | 690 |
| | (313) | (314) | (315) | (316) | (317) | (318) | (303) | (319) |
| mAb10996 | 708 | 24 | 26 | 711 | 713 | 129 | 55 | 715 |
| | (320) | (97) | (98) | (321) | (322) | (137) | (109) | (323) |
| mAb10997 | 708 | 24 | 26 | 711 | 713 | 129 | 55 | 715 |
| | (320) | (97) | (98) | (321) | (322) | (137) | (109) | (323) |
| mAb10998 | 723 | 187 | 26 | 725 | 727 | 129 | 55 | 729 |
| | (324) | (154) | (98) | (325) | (326) | (137) | (109) | (327) |
| mAb10999 | 723 | 187 | 26 | 725 | 727 | 129 | 55 | 729 |
| | (324) | (154) | (98) | (325) | (326) | (137) | (109) | (327) |
| mAb11000 | 737 | 24 | 26 | 739 | 741 | 743 | 55 | 745 |
| | (328) | (97) | (98) | (329) | (340) | (341) | (109) | (342) |
| mAb11001 | 737 | 24 | 26 | 739 | 741 | 743 | 55 | 745 |
| | (328) | (97) | (98) | (329) | (340) | (341) | (109) | (342) |
| mAb11002 | 753 | 24 | 26 | 755 | 713 | 129 | 55 | 715 |
| | (343) | (97) | (98) | (344) | (322) | (137) | (109) | (323) |
| mAb11003 | 753 | 24 | 26 | 755 | 713 | 129 | 55 | 715 |
| | (343) | (97) | (98) | (344) | (322) | (137) | (109) | (323) |
| mAb10914 | 44 | 46 | 26 | 49 | 51 | 53 | 55 | 57 |
| | (104) | (105) | (98) | (106) | (107) | (108) | (109) | (110) |
| mAb11004 | 764 | 766 | 499 | 768 | 770 | 91 | 55 | 772 |
| | (345) | (346) | (254) | (347) | (348) | (122) | (109) | (349) |
| mAb11005 | 764 | 766 | 499 | 768 | 770 | 91 | 55 | 772 |
| | (345) | (346) | (254) | (347) | (348) | (122) | (109) | (349) |
| mAb11006 | 780 | 782 | 26 | 784 | 786 | 53 | 55 | 788 |
| | (350) | (351) | (98) | (352) | (353) | (108) | (109) | (354) |
| mAb11007 | 780 | 782 | 26 | 784 | 786 | 53 | 55 | 788 |
| | (350) | (351) | (98) | (352) | (353) | (108) | (109) | (354) |
| mAb11008 | 796 | 24 | 26 | 798 | 800 | 53 | 55 | 802 |
| | (355) | (97) | (98) | (356) | (357) | (108) | (109) | (358) |
| mAb11009 | 796 | 24 | 26 | 798 | 800 | 53 | 55 | 802 |
| | (355) | (97) | (98) | (356) | (357) | (108) | (109) | (358) |
| mAb11010 | 810 | 812 | 814 | 816 | 818 | 129 | 820 | 822 |
| | (359) | (360) | (361) | (362) | (363) | (137) | (364) | (365) |
| mAb11011 | 810 | 812 | 814 | 816 | 818 | 129 | 820 | 822 |
| | (359) | (360) | (361) | (362) | (363) | (137) | (364) | (365) |

In some embodiments, the spike protein ABDs comprise an amino acid sequence set forth in Table 4 below. In some aspects, the spike protein ABD comprises both heavy and light chain CDRs of an antibody set forth in Table 4 below. In certain aspects, a spike protein ABD comprises a VH having the amino acid sequence of the VH of an antibody set forth in Table 4 and a VL having the amino acid sequence of the VL of the same antibody as set forth in Table 4. In other aspects, a spike protein ABD comprises a VH having the amino acid sequence of the VH of an antibody set forth in Table 4 and a universal light chain VL sequence. The initial sequence identifiers for Table 4 are in relation to the sequence listing in WO 2023/287875A1, which sequence identifiers are incorporated by reference herein, while sequence identifiers presented parenthetically are those of the disclosure.

TABLE 4
SEQ ID NO in WO2023/287875A1 (SEQ ID NO of the disclosure)

| Antibody | | | | | | | | |
|---|---|---|---|---|---|---|---|---|
| Designation | VH | CDR-H1 | CDR-H2 | CDR-H3 | VL | CDR-L1 | CDR-L2 | CDR-L3 |
| mAb15163 | 2 | 4 | 6 | 8 | 10 | 12 | 14 | 16 |
| | (366) | (367) | (368) | (369) | (370) | (371) | (372) | (373) |
| mAb15164 | 22 | 24 | 26 | 28 | 30 | 32 | 34 | 36 |
| | (374) | (375) | (376) | (377) | (378) | (379) | (380) | (381) |
| mAb15165 | 42 | 44 | 26 | 47 | 49 | 51 | 34 | 36 |
| | (382) | (383) | (376) | (384) | (385) | (386) | (380) | (381) |
| mAb15166 | 58 | 60 | 62 | 64 | 66 | 51 | 68 | 36 |
| | (387) | (388) | (389) | (390) | (391) | (386) | (392) | (381) |
| mAb15167 | 74 | 76 | 78 | 80 | 82 | 84 | 86 | 88 |
| | (393) | (394) | (395) | (396) | (397) | (398) | (399) | (400) |
| mAb15170 | 94 | 96 | 98 | 100 | 102 | 104 | 106 | 108 |
| | (401) | (402) | (403) | (404) | (405) | (406) | (407) | (408) |
| mAb14296 | 114 | 116 | 118 | 120 | 122 | 124 | 126 | 128 |
| | (409) | (410) | (411) | (412) | (413) | (414) | (415) | (417) |
| mAb14297 | 134 | 136 | 138 | 140 | 142 | 144 | 126 | 146 |
| | (418) | (419) | (420) | (421) | (422) | (423) | (415) | (424) |
| mAb14312 | 152 | 154 | 156 | 158 | 160 | 162 | 164 | 166 |
| | (425) | (426) | (427) | (428) | (429) | (430) | (431) | (432) |
| mAb14313 | 172 | 174 | 176 | 178 | 180 | 182 | 184 | 186 |
| | (433) | (434) | (435) | (436) | (437) | (438) | (439) | (440) |
| mAb14314 | 192 | 194 | 196 | 198 | 200 | 202 | 204 | 206 |
| | (441) | (442) | (443) | (444) | (445) | (446) | (447) | (448) |
| mAb14315 | 212 | 214 | 216 | 218 | 220 | 222 | 126 | 224 |
| | (449) | (450) | (451) | (452) | (453) | (454) | (415) | (455) |
| mAb14316 | 230 | 232 | 234 | 236 | 238 | 240 | 242 | 244 |
| | (456) | (457) | (458) | (459) | (460) | (461) | (462) | (463) |
| mAb15150 | 250 | 252 | 254 | 256 | 258 | 260 | 262 | 264 |
| | (464) | (465) | (466) | (467) | (468) | (469) | (470) | (471) |
| mAb15151 | 270 | 272 | 274 | 276 | 278 | 280 | 106 | 282 |
| | (472) | (473) | (474) | (475) | (476) | (477) | (407) | (478) |
| mAb15156 | 288 | 290 | 292 | 294 | 296 | 298 | 300 | 302 |
| | (479) | (480) | (481) | (482) | (483) | (484) | (485) | (486) |
| mAb15157 | 308 | 310 | 312 | 314 | 316 | 318 | 320 | 322 |
| | (487) | (488) | (489) | (490) | (491) | (492) | (493) | (494) |
| mAb15158 | 328 | 330 | 332 | 334 | 336 | 338 | 106 | 340 |
| | (495) | (496) | (497) | (498) | (499) | (500) | (407) | (501) |
| mAb15159 | 346 | 96 | 98 | 350 | 352 | 354 | 106 | 356 |
| | (752) | (402) | (403) | (502) | (503) | (504) | (407) | (505) |
| mAb15160 | 362 | 364 | 366 | 368 | 370 | 372 | 106 | 374 |
| | (506) | (507) | (508) | (509) | (510) | (511) | (407) | (512) |
| mAb15161 | 380 | 382 | 384 | 386 | 388 | 390 | 392 | 394 |
| | (513) | (514) | (515) | (516) | (517) | (518) | (519) | (520) |
| mAb15162 | 400 | 402 | 98 | 405 | 407 | 409 | 242 | 411 |
| | (521) | (522) | (403) | (523) | (524) | (525) | (462) | (526) |
| mAb14280 | 417 | 419 | 421 | 423 | 425 | 427 | 14 | 429 |
| | (527) | (528) | (529) | (530) | (531) | (532) | (372) | (533) |
| mAb14281 | 435 | 437 | 138 | 440 | 442 | 444 | 446 | 448 |
| | (534) | (535) | (420) | (536) | (537) | (538) | (539) | (540) |
| mAb14282 | 454 | 456 | 458 | 460 | 462 | 84 | 465 | 467 |
| | (541) | (542) | (543) | (544) | (545) | (398) | (546) | (547) |
| mAb14283 | 473 | 475 | 477 | 479 | 481 | 483 | 485 | 487 |
| | (548) | (549) | (550) | (551) | (552) | (553) | (554) | (555) |
| mAb14284 | 493 | 495 | 497 | 499 | 501 | 503 | 505 | 507 |
| | (556) | (557) | (558) | (559) | (560) | (561) | (562) | (563) |
| mAb14285 | 513 | 515 | 517 | 519 | 521 | 523 | 525 | 36 |
| | (564) | (565) | (566) | (567) | (568) | (569) | (570) | (381) |
| mAb14286 | 531 | 533 | 535 | 537 | 539 | 483 | 542 | 544 |
| | (571) | (572) | (573) | (574) | (575) | (553) | (576) | (577) |
| mAb14287 | 550 | 552 | 554 | 556 | 558 | 84 | 14 | 560 |
| | (578) | (579) | (580) | (581) | (582) | (398) | (372) | (583) |
| mAb14288 | 566 | 568 | 570 | 572 | 574 | 576 | 578 | 580 |
| | (584) | (585) | (586) | (587) | (588) | (589) | (590) | (591) |
| mAb14289 | 586 | 588 | 590 | 592 | 594 | 596 | 126 | 598 |
| | (592) | (593) | (594) | (595) | (596) | (597) | (415) | (598) |
| mAb14290 | 604 | 606 | 608 | 610 | 612 | 614 | 126 | 616 |
| | (599) | (600) | (601) | (602) | (603) | (604) | (415) | (605) |
| mAb14291 | 622 | 624 | 626 | 628 | 630 | 632 | 634 | 636 |
| | (606) | (607) | (608) | (609) | (610) | (611) | (612) | (613) |
| mAb14292 | 642 | 644 | 646 | 648 | 650 | 652 | 634 | 655 |
| | (614) | (615) | (616) | (617) | (618) | (619) | (612) | (620) |
| mAb14293 | 661 | 663 | 665 | 667 | 669 | 124 | 126 | 671 |
| | (621) | (622) | (623) | (624) | (625) | (414) | (415) | (626) |
| mAb14295 | 677 | 679 | 78 | 682 | 684 | 686 | 126 | 688 |
| | (627) | (628) | (395) | (629) | (630) | (631) | (415) | (632) |
| mAb13459 | 694 | 696 | 698 | 700 | 702 | 704 | 706 | 708 |
| | (633) | (634) | (635) | (636) | (637) | (638) | (639) | (640) |
| mAb14230 | 714 | 696 | 716 | 718 | 720 | 722 | 126 | 724 |
| | (641) | (634) | (642) | (643) | (644) | (645) | (415) | (646) |
| mAb14231 | 730 | 732 | 734 | 736 | 738 | 740 | 106 | 742 |
| | (647) | (648) | (649) | (650) | (651) | (652) | (407) | (653) |
| mAb14232 | 748 | 750 | 497 | 752 | 754 | 756 | 505 | 758 |
| | (654) | (655) | (558) | (656) | (657) | (658) | (562) | (659) |
| mAb14233 | 764 | 766 | 768 | 770 | 772 | 774 | 776 | 778 |
| | (660) | (661) | (662) | (663) | (664) | (665) | (666) | (667) |
| mAb14234 | 784 | 786 | 788 | 790 | 792 | 794 | 796 | 798 |
| | (668) | (669) | (670) | (671) | (672) | (673) | (674) | (675) |
| mAb14235 | 804 | 806 | 497 | 808 | 810 | 812 | 505 | 814 |
| | (676) | (677) | (558) | (678) | (679) | (680) | (562) | (681) |
| mAb14247 | 820 | 822 | 497 | 825 | 827 | 756 | 14 | 829 |
| | (682) | (683) | (558) | (684) | (685) | (658) | (372) | (686) |
| mAb14248 | 835 | 837 | 839 | 841 | 843 | 845 | 847 | 849 |
| | (687) | (688) | (689) | (690) | (691) | (692) | (693) | (694) |
| mAb14249 | 855 | 857 | 859 | 861 | 863 | 865 | 106 | 867 |
| | (695) | (696) | (697) | (698) | (699) | (700) | (407) | (701) |
| mAb14255 | 873 | 76 | 876 | 878 | 880 | 84 | 86 | 36 |
| | (702) | (394) | (703) | (704) | (705) | (398) | (399) | (381) |
| mAb14256 | 887 | 889 | 891 | 893 | 895 | 897 | 164 | 899 |
| | (706) | (707) | (708) | (709) | (710) | (711) | (431) | (712) |
| mAb14257 | 905 | 154 | 908 | 910 | 912 | 914 | 916 | 166 |
| | (713) | (426) | (714) | (715) | (716) | (717) | (718) | (432) |
| mAb14258 | 922 | 924 | 926 | 928 | 930 | 576 | 933 | 935 |
| | (719) | (720) | (721) | (722) | (723) | (589) | (724) | (725) |
| mAb14259 | 941 | 943 | 945 | 947 | 949 | 951 | 634 | 655 |
| | (726) | (727) | (728) | (729) | (730) | (731) | (612) | (620) |
| mAb14260 | 957 | 959 | 961 | 963 | 965 | 967 | 969 | 971 |
| | (732) | (733) | (734) | (735) | (736) | (737) | (738) | (739) |
| mAb13457 | 694 | 696 | 698 | 700 | 977 | 704 | 706 | 979 |
| | (633) | (634) | (635) | (636) | (740) | (638) | (639) | (741) |
| mAb13458 | 694 | 696 | 698 | 700 | 983 | 704 | 706 | 985 |
| | (633) | (634) | (635) | (636) | (742) | (638) | (639) | (743) |
| mAb14294 | 989 | 991 | 993 | 995 | 997 | 999 | 1001 | 1003 |
| | (744) | (745) | (746) | (747) | (748) | (749) | (750) | (751) |
| mAb17090 | 493 | 495 | 497 | 499 | 501 | 503 | 505 | 507 |
| | (556) | (557) | (558) | (559) | (560) | (561) | (562) | (563) |
| mAb15160_2 | 362 | 364 | 366 | 368 | 370 | 372 | 106 | 374 |
| | (506) | (507) | (508) | (509) | (510) | (511) | (407) | (512) |

In some embodiments, a spike protein ABD of a multivalent anti-spike protein binding molecule comprises the heavy and light chain CDRs of antibody “mAb14287” as set forth in Table 4. Accordingly, in some embodiments, the spike protein ABD comprises a VH comprising CDR-H1, CDR-H2, and CDR-H3 having the amino acid sequences of SEQ ID NOs: 579, 580, and 581, respectively, and a VL comprising CDR-L1, CDR-L2, and CDR-L3 having the amino acid sequences of SEQ ID NOs: 398, 372, and 583, respectively. In some embodiments, the spike protein ABD comprises a VH having at least 90%, at least 95%, at least 96%, at least 97%, at least 98%, at least 99%, or 100% sequence identity to the amino acid sequence of SEQ ID NO:578 and a VL having at least 90%, at least 95%, at least 96%, at least 97%, at least 98%, at least 99%, or 100% sequence identity to the amino acid sequence of SEQ ID NO:582. In some embodiments, the spike protein ABD comprises a VH comprising the amino acid sequence of SEQ ID NO:578 and a VL comprising the amino acid sequence of SEQ ID NO:582.

In some embodiments, a spike protein ABD of a multivalent anti-spike protein binding molecule comprises the heavy and light chain CDRs of antibody “mAb15160” as set forth in Table 4. Accordingly, in some embodiments, the spike protein ABD comprises a VH comprising CDR-H1, CDR-H2, and CDR-H3 having the amino acid sequences of SEQ ID NOs: 507, 508, and 509, respectively, and a VL comprising CDR-L1, CDR-L2, and CDR-L3 having the amino acid sequences of SEQ ID NOs: 511, 407, and 512, respectively. In some embodiments, the spike protein ABD comprises a VH having at least 90%, at least 95%, at least 96%, at least 97%, at least 98%, at least 99%, or 100% sequence identity to the amino acid sequence of SEQ ID NO:506 and a VL having at least 90%, at least 95%, at least 96%, at least 97%, at least 98%, at least 99%, or 100% sequence identity to the amino acid sequence of SEQ ID NO:510. In some embodiments, the spike protein ABD comprises a VH comprising the amino acid sequence of SEQ ID NO:506 and a VL comprising the amino acid sequence of SEQ ID NO:510.

In some embodiments, a spike protein ABD of a multivalent anti-spike protein binding molecule comprises the heavy and light chain CDRs of antibody “mAb14315” as set forth in Table 4. Accordingly, in some embodiments, the spike protein ABD comprises a VH comprising CDR-H1, CDR-H2, and CDR-H3 having the amino acid sequences of SEQ ID NOs: 450, 451, and 452, respectively, and a VL comprising CDR-L1, CDR-L2, and CDR-L3 having the amino acid sequences of SEQ ID NOs: 454, 415, and 455, respectively. In some embodiments, the spike protein ABD comprises a VH having at least 90%, at least 95%, at least 96%, at least 97%, at least 98%, at least 99%, or 100% sequence identity to the amino acid sequence of SEQ ID NO:449 and a VL having at least 90%, at least 95%, at least 96%, at least 97%, at least 98%, at least 99%, or 100% sequence identity to the amino acid sequence of SEQ ID NO:453. In some embodiments, the spike protein ABD comprises a VH comprising the amino acid sequence of SEQ ID NO:449 and a VL comprising the amino acid sequence of SEQ ID NO:453.

TABLE S
Sequences of Tables 3 and 4 Excluded
from ST.26-Formatted Sequence Listing

| SEQ ID NO | Sequence |
|---|---|
| 94 | DNN |
| 102 | KAS |
| 109 | AAS |
| 130 | WAS |
| 145 | GVS |
| 157 | DAS |
| 195 | GAS |
| 279 | GNS |
| 296 | GNT |
| 303 | DVS |
| 311 | SNN |
| 364 | TAS |
| 372 | EVS |
| 380 | EGN |
| 392 | EGT |
| 399 | EDS |
| 407 | AAS |
| 415 | GAS |
| 431 | GNS |
| 439 | SND |
| 447 | DND |
| 462 | DAS |
| 470 | GAT |
| 485 | SDN |
| 493 | VNN |
| 519 | KAS |
| 539 | DKN |
| 546 | ELT |
| 554 | DVT |
| 562 | EVT |
| 570 | EGS |
| 576 | DVS |
| 590 | ENN |
| 612 | LGS |
| 639 | SAS |
| 666 | KDS |
| 674 | GNT |
| 693 | KIS |
| 718 | GHT |
| 724 | RNN |
| 738 | WAS |
| 750 | GAS |

### 6.3. Spike Protein Antigen Binding Domain Formats

In certain aspects, the multivalent anti-spike protein binding molecules of the disclosure comprise an ABD of an anti-spike protein antibody that retains specific binding to an antigenic determinant. In one embodiment, the spike protein ABD is a naturally occurring (e.g., by protease cleavage) or engineered fragment of an immunoglobulin. Antibody fragments include, but are not limited to, VH (or VH) fragments, VL (or VL) fragments, Fab fragments, F(ab′)2 fragments, scFv fragments, Fv fragments, minibodies, diabodies, triabodies, and tetrabodies.

In some embodiments, the spike protein ABD is in the form of a Fab or an scFv.

### 6.3.1. Fab

Fab domains were traditionally produced by proteolytic cleavage of immunoglobulin molecules using enzymes such as papain. The Fab domains can comprise constant domain and variable region sequences from any suitable species, and thus can be murine, chimeric, human or humanized.

Fab domains typically comprise a CH1 domain attached to a VH domain which pairs with a CL domain attached to a VL domain. In a wild-type immunoglobulin, the VH domain is paired with the VL domain to constitute the Fv region, and the CH1 domain is paired with the CL domain to further stabilize the binding site. A disulfide bond between the two constant domains can further stabilize the Fab domain.

For the anti-spike protein binding antibodies of the disclosure that are not homodimeric, particularly when the light chains of the anti-spike protein antibody are not common or universal light chains, it is advantageous to use Fab heterodimerization strategies to permit the correct association of Fab domains belonging to the same antigen-binding domain and minimize aberrant pairing of Fab domains belonging to different antigen-binding domains. For example, the Fab heterodimerization strategies shown in Table 5 below can be used:

TABLE 5
Fab Heterodimerization Strategies

| STRATEGY | VH | CH1 | VL | CL | REFERENCE |
|---|---|---|---|---|---|
| CrossMabCH1- | WT | CL domain | WT | CH1 domain | Schaefer et al., 2011, |
| CL | | | | | Cancer Cell 2011; |
| | | | | | 20: 472-86; |
| | | | | | PMID: 22014573. |
| orthogonal Fab | 39K, 62E | H172A, F174G | 1R, 38D, (36F) | L135Y, S176W | Lewis et al., 2014, Nat |
| VHVRD1CH1CRD2 - | | | | | Biotechnol 32: 191-8 |
| VLVRD1CACRD2 | | | | | |
| orthogonal Fab | 39Y | WT | 38R | WT | Lewis et al., 2014, Nat |
| VHVRD2CH1wt - | | | | | Biotechnol 32: 191-8 |
| VLVRD2Cλwt | | | | | |
| TCR CαCβ | 39K | TCR Cα | 38D | TCR Cβ | Wu et al., 2015, MABD |
| | | | | | 7: 364-76 |
| CR3 | WT | T192E | WT | N137K, S114A | Golay at al., 2016, J |
| | | | | | Immunol 196: 3199-211. |
| MUT4 | WT | L143Q, S188V | WT | V133T, S176V | Golay at al., 2016, J |
| | | | | | Immunol 196: 3199-211. |
| DuetMab | WT | F126C | WT | S121C | Mazor et al., 2015, |
| | | | | | MABD 7: 377-89; Mazor |
| | | | | | et al., 2015, MABD |
| | | | | | 7: 461-669. |
| Domain | WT | CH3 + knob or | WT | CH3 + hole or | Wozniak-Knopp et al., |
| exchanged | | hole mutation | | knob mutation | 2018, PloS |
| | | | | | ONE13(4): e0195442 |

Accordingly, in certain embodiments, correct association between the two polypeptides of a Fab is promoted by exchanging the VL and VH domains of the Fab for each other or exchanging the CH1 and CL domains for each other, e.g., as described in WO 2009/080251.

Correct Fab pairing can also be promoted by introducing one or more amino acid modifications in the CH1 domain and one or more amino acid modifications in the CL domain of the Fab and/or one or more amino acid modifications in the VH domain and one or more amino acid modifications in the VL domain. The amino acids that are modified are typically part of the VH:VL and CH1:CL interface such that the Fab components preferentially pair with each other rather than with components of other Fabs.

In one embodiment, the one or more amino acid modifications are limited to the conserved framework residues of the variable (VH, VL) and constant (CH1, CL) domains as indicated by the Kabat numbering of residues. Almagro, 2008, Frontiers In Bioscience 13:1619-1633 provides a definition of the framework residues on the basis of Kabat, Chothia, and IMGT numbering schemes.

In one embodiment, the modifications introduced in the VH and CH1 and/or VL and CL domains are complementary to each other. Complementarity at the heavy and light chain interface can be achieved on the basis of steric and hydrophobic contacts, electrostatic/charge interactions or a combination of the variety of interactions. The complementarity between protein surfaces is broadly described in the literature in terms of lock and key fit, knob into hole, protrusion and cavity, donor and acceptor etc., all implying the nature of structural and chemical match between the two interacting surfaces.

In one embodiment, the one or more introduced modifications introduce a new hydrogen bond across the interface of the Fab components. In one embodiment, the one or more introduced modifications introduce a new salt bridge across the interface of the Fab components. Exemplary substitutions are described in WO 2014/150973 and WO 2014/082179, the contents of which are hereby incorporated by reference.

In some embodiments, the Fab domain comprises a 192E substitution in the CH1 domain and 114A and 137K substitutions in the CL domain, which introduces a salt-bridge between the CH1 and CL domains (see, e.g., Golay et al., 2016, J Immunol 196:3199-211).

In some embodiments, the Fab domain comprises a 143Q and 188V substitutions in the CH1 domain and 113T and 176V substitutions in the CL domain, which serves to swap hydrophobic and polar regions of contact between the CH1 and CL domain (see, e.g., Golay et al., 2016, J Immunol 196:3199-211).

In some embodiments, the Fab domain can comprise modifications in some or all of the VH, CH1, VL, CL domains to introduce orthogonal Fab interfaces which promote correct assembly of Fab domains (Lewis et al., 2014, Nature Biotechnology 32:191-198). In an embodiment, 39K, 62E modifications are introduced in the VH domain, H172A, F174G modifications are introduced in the CH1 domain, 1 R, 38D, (36F) modifications are introduced in the VL domain, and L135Y, S176W modifications are introduced in the CL domain. In another embodiment, a 39Y modification is introduced in the VH domain and a 38R modification is introduced in the VL domain.

Fab domains can also be modified to replace the native CH1:CL disulfide bond with an engineered disulfide bond, thereby increasing the efficiency of Fab component pairing. For example, an engineered disulfide bond can be introduced by introducing a 126C in the CH1 domain and a 121 C in the CL domain (see, e.g., Mazor et al., 2015, MABD 7:377-89).

Fab domains can also be modified by replacing the CH1 domain and CL domain with alternative domains that promote correct assembly. For example, Wu et al., 2015, MABD 7:364-76, describes substituting the CH1 domain with the constant domain of the T cell receptor and substituting the CL domain with the b domain of the T cell receptor, and pairing these domain replacements with an additional charge-charge interaction between the VL and VH domains by introducing a 38D modification in the VL domain and a 39K modification in the VH domain.

6.3.2. scFv

Single chain Fv or “scFv” antibody fragments comprise the VH and VL domains of an antibody in a single polypeptide chain, are capable of being expressed as a single chain polypeptide and retain the specificity of the intact antibodies from which they are derived. Generally, the scFv polypeptide further comprises a polypeptide linker between the VH and VL domain that enables the scFv to form the desired structure for target binding. Examples of linkers suitable for connecting the VH and VL chains of an scFv are the linkers identified in Section 6.5.

Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The scFv can comprise VH and VL sequences from any suitable species, such as murine, human or humanized VH and VL sequences.

To create an scFv-encoding nucleic acid, the VH and VL-encoding DNA fragments are operably linked to another fragment encoding a linker, e.g., encoding any of the linkers described in Section 6.5 (typically a repeat of a sequence containing the amino acids glycine and serine, such as the amino acid sequence (Gly4˜Ser)3, such that the VH and VL sequences can be expressed as a contiguous single-chain protein, with the VL and VH regions joined by the flexible linker (see, e.g., Bird et al., 1988, Science 242:423-426; Huston et al., 1988, Proc. Natl. Acad. Sci. USA 85:5879-5883; McCafferty et al., 1990, Nature 348:552-554).

### 6.4. Multimerization Moiety

In some embodiments, the multivalent anti-spike protein binding molecules of the disclosure include one or more multimerization moieties, for example one or more multimerization moieties that are or comprise an Fc domain. In certain embodiments, an multivalent anti-spike protein binding molecule of the disclosure comprises a single multimerization moiety (e.g., a single Fc domain) and/or an multivalent anti-spike protein binding molecule of the disclosure comprises two or more multimerization moieties (e.g., two or more Fc domains that can associate to form an Fc region). In some embodiments, the ACE fusion protein is a pentamer or hexamer of five or six IgM-derived dimeric Fc regions, for example as described in Section 6.4.1.

### 6.4.1. Fc Domains

The multivalent anti-spike protein binding molecules of the disclosure can include an Fc domain, or a pair of Fc domains that associate to form an Fc region, derived from any suitable species operably linked to an ACE2 moiety. In one embodiment the Fc domain is derived from a human Fc domain. In preferred embodiments, the ACE2 moiety is fused to an IgM Fc domain.

The Fc domains that can be incorporated into multivalent anti-spike protein binding molecules can be derived from any suitable class of antibody, including IgA (including subclasses IgA1 and IgA2), IgD, IgE, IgG (including subclasses IgG1, IgG2, IgG3 and IgG4), and IgM. In one embodiment, the Fc domain is derived from IgM.

In native antibodies, the heavy chain Fc domain of IgA, IgD and IgG is composed of two heavy chain constant domains (Cμ2 and Cμ3) and that of IgE and IgM is composed of three heavy chain constant domains (Cμ2, Cμ3 and Cμ4). These dimerize to create an Fc region.

In the multivalent anti-spike protein binding molecules of the present disclosure, the Fc region, and/or the Fc domains within it, can comprise heavy chain constant domains from one or more different classes of antibody, for example one, two or three different classes.

### 6.4.2. IgM Fc Domains

In some embodiments, the multivalent anti-spike protein binding molecules of the present disclosure comprise Fc domains derived from IgM. IgM occurs naturally in humans as covalent multimers of heavy chain (H) light chain (L) assemblies forming a common H2L2 antibody unit. In addition to the heavy and light chains, IgMs also possess a third chain, known as the joining (J)-chain (Keyt et al., 2020, Antibodies. 9(4):53). IgM occurs as a pentamer when it has incorporated a J chain, or as a hexamer when it lacks a J chain.

The heavy chain constant domains for use in producing an IgM Fc region for the multivalent anti-spike protein binding molecules of the present disclosure may include variants of the naturally occurring constant domains described above. In one example, the Fc region of the present disclosure comprises at least one constant domain that varies in sequence from the wildtype constant domain. It will be appreciated that the variant constant domains may be longer or shorter than their counterpart wild type constant domains.

The heavy chains of IgM possess an 18 amino acid extension to the C-terminal constant domain, known as a tailpiece. The tailpiece includes a cysteine residue that forms a disulfide bond between heavy chains in the polymer and is believed to have an important role in polymerization. The tailpiece also contains a glycosylation site. In certain embodiments, the multivalent anti-spike protein binding molecules of the present disclosure comprise a tailpiece.

IgM assembly typically starts with the association of a heavy (H) and a light (L) chain into a H-L arrangement, which then dimerizes to form H2L2 subunits. A critical site for this intra-subunit assembly is Cys337, which forms a disulfide bond between two Cμ2 domains and stabilizes the H2L2. Next, these subunits are brought together by disulfide bridges to form multimers. A residue involved in this multimerization is Cys575 on tail domains of Cμ4, which forms disulfide bonds and enables noncovalent Cμ4 interactions. Another important residue is Cys414 on Cμ3, which further connects two Cμ3 domains of neighboring H2L2 subunits, in series to the disulfide bond between Cys337 residues of Cμ2. In the presence of the J chain, IgM assembly results in a pentamer, in which Cys337 disulfide bonds is in series with both Cys414 disulfide bonds and Cys575 disulfide bonds (Pasalic et al., 2017, Proc. Nat'l Acad. Sci USA 114 (41) E8575-E8584; Keyt et al., 2020, Antibodies. 9(4):53; Casali, 1998. Encyclopedia of Immunology (2nd Ed), p1212-1217). For an IgM assembly to include a J chain, a J chain polypeptide needs to be co-expressed with the polypeptides encoding H2L2 subunits domains.

In certain embodiments, the multimerization moiety provided by this disclosure is a pentameric or hexameric binding molecule that includes dimeric IgM heavy chain constant regions, or multimerizing fragments thereof.

An exemplary sequence of a full-length human IgM heavy chain constant domain is reproduced below.

(SEQ ID NO: 3)
GSASAPTLFP LVSCENSPSD TSSVAVGCLA QDFLPDSITF
SWKYKNNSDI SSTRGFPSVL RGGKYAATSQ VLLPSKDVMQ
GTDEHVVCKV QHPNGNKEKN VPLPVIAELP PKVSVFVPPR
DGFFGNPRKS KLICQATGES PRQIQVSWLR EGKQVGSGVT
TDQVQAEAKE SGPTTYKVTS TLTIKESDWL SQSMFTCRVD
HRGLTFQQNA SSMCVPDQDT AIRVFAIPPS FASIFLTKST
KLTCLVTDLT TYDSVTISWT RQNGEAVKTH TNISESHPNA
TFSAVGEASI CEDDWNSGER FTCTVTHTDL PSPLKQTISR
PKGVALHRPD VYLLPPAREQ LNLRESATIT CLVTGFSPAD
VFVQWMQRGQ PLSPEKYVTS APMPEPQAPG RYFAHSILTV
SEEEWNTGET YTCVVAHEAL PNRVTERTVD KSTGKPTLYN
VSLVMSDTAG TCY

While not wishing to be bound by theory, the assembly of dimeric IgM Fc regions into a pentameric or hexameric structure is thought to involve at least the Cμ4, and/or tailpiece (TP) domains (Braathen, R., et al., 2002. J. Biol. Chem. 277:42755-42762). Accordingly, a multimerization moiety based on the IgM Fc domain typically includes at least the Cμ4 and/or TP domain sequences.

An IgM heavy chain constant domain can additionally include a Cμ3 domain or a fragment thereof, a Cμ2 domain or a fragment thereof, and/or other IgM or other immunoglobulin heavy chain domains.

Exemplary sequences of human IgM heavy chain constant domains are reproduced in Table 6 below.

TABLE 6
Exemplary Configurations of IgM Heavy Chain Constant Domains

| | | SEQ ID |
|---|---|---|
| Domain(s) | Sequence | NO |
| Cμ4 | DVYLLPPAREQLNLRESATITCLVTGFSPADVFVQWMQRG | 4 |
| | QPLSPEKYVTSAPMPEPQAPGRYFAHSILTVSEEEWNTGE | |
| | TYTCVVAHEALPNRVTERTVD | |
| Cμ4-TP | DVYLLPPAREQLNLRESATITCLVTGFSPADVEVQWMQRG | 5 |
| | QPLSPEKYVTSAPMPEPQAPGRYFAHSILTVSEEEWNTGE | |
| | TYTCVVAHEALPNRVTERTVDKSTGKPTLYNVSLVMSDTA | |
| | GTCY | |
| Cμ3-Cμ4 | VFAIPPSFASIFLTKSTKLTCLVTDLTTYDSVTISWTRQN | 6 |
| | GEAVKTHTNISESHPNATFSAVGEASICEDDWNSGERFTC | |
| | TVTHTDLPSPLKQTISRPKGVALHRPDVYLLPPAREQLNL | |
| | RESATITCLVTGFSPADVFVQWMQRGQPLSPEKYVTSAPM | |
| | PEPQAPGRYFAHSILTVSEEEWNTGETYTCVVAHEALPNR | |
| | VTERTVD | |
| Cμ3-Cμ4-TP | VFAIPPSFASIFLTKSTKLTCLVTDLTTYDSVTISWTRQN | 7 |
| | GEAVKTHTNISESHPNATFSAVGEASICEDDWNSGERFTC | |
| | TVTHTDLPSPLKQTISRPKGVALHRPDVYLLPPAREQLNL | |
| | RESATITCLVTGFSPADVEVQWMQRGQPLSPEKYVTSAPM | |
| | PEPQAPGRYFAHSILTVSEEEWNTGETYTCVVAHEALPNR | |
| | VTERTVDKSTGKPTLYNVSLVMSDTAGTCY | |
| Cμ2-Cμ3-Cμ4 | SVFVPPRDGFFGNPRKSKLICQATGESPRQIQVSWLREGK | 8 |
| | QVGSGVTTDQVQAEAKESGPTTYKVTSTLTIKESDWLSQS | |
| | MFTCRVDHRGLTFQQNASSMCVPDQDTAIRVFAIPPSFAS | |
| | IFLTKSTKLTCLVTDLTTYDSVTISWTRQNGEAVKTHTNI | |
| | SESHPNATESAVGEASICEDDWNSGERFTCTVTHTDLPSP | |
| | LKQTISRPKGVALHRPDVYLLPPAREQLNLRESATITCLV | |
| | TGFSPADVFVQWMQRGQPLSPEKYVTSAPMPEPQAPGRYF | |
| | AHSILTVSEEEWNTGETYTCVVAHEALPNRVTERTVD | |
| Cμ2-Cμ3-Cμ4- | SVFVPPRDGFFGNPRKSKLICQATGESPRQIQVSWLREGK | 9 |
| TP | QVGSGVTTDQVQAEAKESGPTTYKVTSTLTIKESDWLSQS | |
| | MFTCRVDHRGLTFQQNASSMCVPDQDTAIRVFAIPPSFAS | |
| | IFLTKSTKLTCLVTDLTTYDSVTISWTRQNGEAVKTHTNI | |
| | SESHPNATFSAVGEASICEDDWNSGERFTCTVTHTDLPSP | |
| | LKQTISRPKGVALHRPDVYLLPPAREQLNLRESATITCLV | |
| | TGFSPADVFVQWMQRGQPLSPEKYVTSAPMPEPQAPGRYF | |
| | AHSILTVSEEEWNTGETYTCVVAHEALPNRVTERTVDKST | |
| | GKPTLYNVSLVMSDTAGTCY | |

In some embodiments, the Fc domain comprises the amino acid sequence of the Cμ4 domain of IgM or an amino acid sequence having at least 85%, at least 90%, at least 93%, at least 95%, at least 98% or at least 99% sequence identity thereto. In some embodiments, the Fc domain comprises an amino acid sequence having at least 85%, at least 90%, at least 93%, at least 95%, at least 98% sequence identity, at least 99% sequence identity or 100% sequence identity to the amino acid sequence SEQ ID NO:4.

In some embodiments, the Fc domain comprises the amino acid sequence of the Cμ4 and tailpiece domains of IgM or an amino acid sequence having at least 85%, at least 90%, at least 93%, at least 95%, at least 98% or at least 99% sequence identity thereto. In some embodiments, the Fc domain comprises an amino acid sequence having at least 85%, at least 90%, at least 93%, at least 95%, at least 98% sequence identity, at least 99% sequence identity or 100% sequence identity to the amino acid sequence of SEQ ID NO:5.

In some embodiments, the Fc domain comprises the amino acid sequence of the Cμ3 and Cμ4 domains of IgM or an amino acid sequence having at least 85%, at least 90%, at least 93%, at least 95%, at least 98% or at least 99% sequence identity thereto. In some embodiments, the Fc domain comprises an amino acid sequence having at least 85%, at least 90%, at least 93%, at least 95%, at least 98% sequence identity, at least 99% sequence identity or 100% sequence identity to the amino acid sequence of SEQ ID NO:6.

In some embodiments, the Fc domain comprises the amino acid sequence of the Cμ3 and Cμ4 and tailpiece domains of IgM or an amino acid sequence having at least 85%, at least 90%, at least 93%, at least 95%, at least 98% or at least 99% sequence identity thereto. In some embodiments, the Fc domain comprises an amino acid sequence having at least 85%, at least 90%, at least 93%, at least 95%, at least 98% sequence identity, at least 99% sequence identity or 100% sequence identity to the amino acid sequence of SEQ ID NO:7.

In further embodiments, the Fc domain comprises the amino acid sequence of the Cμ2, Cμ3, and Cμ4 domains of IgM or an amino acid sequence having at least 85%, at least 90%, at least 93%, at least 95%, at least 98% or at least 99% sequence identity thereto. In some embodiments, the Fc domain comprises an amino acid sequence having at least 85%, at least 90%, at least 93%, at least 95%, at least 98%, at least 99% sequence identity or 100% sequence identity to the amino acid sequence of SEQ ID NO:8.

In yet further embodiments, the Fc domain comprises the amino acid sequence of the Cμ2, Cμ3, Cμ4 and tailpiece domains of IgM or an amino acid sequence having at least 85%, at least 90%, at least 93%, at least 95%, at least 98% or at least 99% sequence identity thereto. In some embodiments, the Fc domain comprises an amino acid sequence having at least 85%, at least 90%, at least 93%, at least 95%, at least 98% sequence identity, at least 99% sequence identity or 100% sequence identity to the amino acid sequence of SEQ ID NO:9.

### 6.4.3. J Chains

A J chain is a small, 137-residue polypeptide, which is associated with IgM via forming disulfide bonds with Cμ4 tailpieces. The incorporation of the J chain into pentameric IgM closes the ring structure by bridging the first and fifth monomeric units, thereby excluding addition of a sixth IgM monomer.

An exemplary amino acid sequence of human mature wild type J chain is reproduced below.

(SEQ ID NO: 1)
QEDERIVLVD NKCKCARITS RIIRSSEDPN EDIVERNIRI
IVPLNNRENI SDPTSPLRTR FVYHLSDLCK KCDPTEVELD
NQIVTATQSN ICDEDSATET CYTYDRNKCY TAVVPLVYGG
ETKMVETALT PDACYPD

In some embodiments, an engineered J chain is incorporated into IgM pentamers. An exemplary amino sequence of human mature engineered J chain is reproduced below.

(SEQ ID NO: 2)
QEDERIVLVD NKCKCARITS RIIRSSEDPN EDIVERNIRI
IVPLNNRENI ADPTSPLRTR FVYHLSDLCK KCDPTEVELD
NQIVTATQSN ICDEDSATET CYTYDRNKCY TAVVPLVYGG
ETKMVETALT PDACYPD

The multivalent anti-spike protein binding molecules (e.g., pentameric multivalent anti-spike protein binding molecules) of the disclosure may further comprise a J chain polypeptide associated with the CH4 tailpieces. In various embodiments, the J chain polypeptide comprises the amino acid sequence of a mature naturally occurring or engineered J chain polypeptide or an amino acid having at least 85%, at least 90%, at least 93%, at least 95%, at least 98% or at least 99% sequence identity thereto. In some embodiments, the J chain polypeptide comprises an amino acid sequence having at least 85%, at least 90%, at least 93%, at least 95% or at least 98% sequence identity, at least 99% sequence identity or 100% sequence identity to the amino acid sequence of SEQ ID NO:1 or SEQ ID NO:2.

### 6.4.3.1. IgG Fc-Linked J Chains

In some embodiments, a multivalent anti-spike protein binding molecule of the disclosure comprises a J chain polypeptide operably linked to an IgG Fc domain, optionally via a polypeptide linker. Examples of linkers suitable for connecting a J chain and an IgG Fc domain are the linkers identified in Section 6.5. In some embodiments, the IgG Fc domain is connected to the N-terminus of the J chain polypeptide. In some embodiments, the IgG Fc domain is connected to the C-terminus of the J chain polypeptide. Examples of such multivalent anti-spike protein binding molecules are illustrated in FIGS. 1C-1E.

In one embodiment, the IgG Fc domain is derived from IgG1, IgG2, IgG3 or IgG4. In one embodiment the Fc IgG domain is derived from IgG1. In one embodiment the Fc domain is derived from IgG4.

Exemplary sequences of IgG Fc domains from IgG1, IgG2, IgG3, and IgG4 are provided in Table Y-1, below.

TABLE Y-1
Fc Sequences

| | | SEQ |
|---|---|---|
| Fc | Sequence | ID NO |
| hIgG1 Fc (amino | APELLGGPSVFLFPPKPKDTLMISRTPEVTCVVVDVSHEDPEVKFNWYVDGVEV | 10 |
| acids 114-330 of | HNAKTKPREEQYNSTYRVVSVLTVLHQDWLNGKEYKCKVSNKALPAPIEKTISK | |
| UniprotKB | AKGQPREPQVYTLPPSRDELTKNQVSLTCLVKGFYPSDIAVEWESNGQPENNYK | |
| P01857-1) | TTPPVLDSDGSFFLYSKLTVDKSRWQQGNVFSCSVMHEALHNHYTQKSLSLSPG | |
| | K | |
| hIgG2 Fc (amino | APPVAGPSVFLFPPKPKDTLMISRTPEVTCVVVDVSHEDPEVQFNWYVDGVEVH | 11 |
| acids 111-326 of | NAKTKPREEQFNSTFRVVSVLTVVHQDWLNGKEYKCKVSNKGLPAPIEKTISKT | |
| UniprotKB | KGQPREPQVYTLPPSREEMTKNQVSLTCLVKGFYPSDISVEWESNGQPENNYKT | |
| P01859-1) | TPPMLDSDGSFFLYSKLTVDKSRWQQGNVFSCSVMHEALHNHYTQKSLSLSPGK | |
| hIgG3 Fc (amino | APELLGGPSVFLFPPKPKDTLMISRTPEVTCVVVDVSHEDPEVQFKWYVDGVEV | 12 |
| acids 161-377 of | HNAKTKPREEQYNSTFRVVSVLTVLHQDWLNGKEYKCKVSNKALPAPIEKTISK | |
| UniprotKB | TKGQPREPQVYTLPPSREEMTKNQVSLTCLVKGFYPSDIAVEWESSGQPENNYN | |
| P01860-1) | TTPPMLDSDGSFFLYSKLTVDKSRWQQGNIFSCSVMHEALHNRFTQKSLSLSPG | |
| | K | |
| hIgG4 Fc (amino | APEFLGGPSVFLFPPKPKDTLMISRTPEVTCVVVDVSQEDPEVQFNWYVDGVEV | 13 |
| acids 111-327 of | HNAKTKPREEQFNSTYRVVSVLTVLHQDWLNGKEYKCKVSNKGLPSSIEKTISK | |
| UniprotKB | AKGQPREPQVYTLPPSQEEMTKNQVSLTCLVKGFYPSDIAVEWESNGQPENNYK | |
| P01861-1) | TTPPVLDSDGSFFLYSRLTVDKSRWQEGNVFSCSVMHEALHNHYTQKSLSLSLG | |
| | K | |

In some embodiments, the IgG Fc domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:10.

In some embodiments, the IgG Fc domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:11.

In some embodiments, the IgG Fc domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:12.

In some embodiments, the IgG Fc domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:13.

In some embodiments, the IgG Fc domain is a non-dimerizing (or “monomeric”) Fc domain, which refers to an Fc domain that has a reduced ability to self-associate relative to a wild type Fc domain, or which lacks the ability to self-associate entirely, e.g., as described in Helm et al., 1996, J. Biol. Chem. 271: 7494-7500 or Ying et al., 2012, J Biol Chem. 287(23): 19399-19408. An example non-dimerizing Fc domain comprises amino acid substitutions in the positions corresponding to T366 and/or Y407 in CH3 (numberings according to Kabat EU index), as described in U.S. Patent Publication No. 2019/0367611, incorporated herein by reference. Particular amino acid substitutions which may be included in a non-dimerizing Fc domain include, for example, L351S, T366R, L368H, P395K, L242C, K334C, L351S, P343C, A431C, L351Y, T366Y, L368A, P395R, F405R, Y407M, K409A, F405E, Y407K, L351K, T366S, P395V, Y407A, and K409Y (numberings according to Kabat EU index). A non-dimerizing Fc domain of the present disclosure may include any 1, 2, 3, 4, 5, 6, 7, 8, 9, or 10 of the abovementioned substitutions, or more.

Exemplary sequences of non-dimerizing Fc domains are provided in Table Y-2, below. Bolded residues show locations of amino acid substitutions relative to wild type human IgG sequence.

TABLE Y-2
Non-dimerizing Fc Sequences

| | | SEQ |
|---|---|---|
| Non-dimerizing Fc | Sequence | ID NO |
| IgG1 mFc (L351S, | APELLGGPSVFLFPPKPKDTLMISRTPEVTCVVVDVSHEDPEVKFNWYV | 14 |
| T366R, L368H, P395K; | DGVEVHNAKTKPREEQYNSTYRVVSVLTVLHQDWLNGKEYKCKVSNKAL | |
| Kabat EU numbering) | PAPIEKTISKAKGQPREPQVYTSPPSRDELTKNQVSLRCHVKGFYPSDI | |
| | AVEWESNGQPENNYKTTKPVLDSDGSFFLYSKLTVDKSRWQQGNVFSCS | |
| | VMHEALHNHYTQKSLSLSPGK | |
| IgG1 smFc (L242C, | APELLGGPSVFCFPPKPKDTLMISRTPEVTCVVVDVSHEDPEVKFNWYV | 15 |
| K334C, L351S, T366R, | DGVEVHNAKTKPREEQYNSTYRVVSVLTVLHQDWLNGKEYKCKVSNKAL | |
| L368H, P395K; Kabat | PAPIECTISKAKGQPREPQVYTSPPSRDELTKNQVSLRCHVKGFYPSDI | |
| EU numbering) | AVEWESNGQPENNYKTTKPVLDSDGSFFLYSKLTVDKSRWQQGNVFSCS | |
| | VMHEALHNHYTQKSLSLSPGK | |
| IgG1 ssmFc (L242C, | APELLGGPSVFCFPPKPKDTLMISRTPEVTCVVVDVSHEDPEVKFNWYV | 16 |
| K334C, P343C, L351S, | DGVEVHNAKTKPREEQYNSTYRVVSVLTVLHQDWLNGKEYKCKVSNKAL | |
| T366R, L368H, P395K, | PAPIECTISKAKGQCREPQVYTSPPSRDELTKNQVSLRCHVKGFYPSDI | |
| A431C; Kabat EU | AVEWESNGQPENNYKTTKPVLDSDGSFFLYSKLTVDKSRWQQGNVFSCS | |
| numbering) | VMHECLHNHYTQKSLSLSPGK | |
| IgG1 mFc.1 (L351Y, | APELLGGPSVFLFPPKPKDTLMISRTPEVTCVVVDVSHEDPEVKFNWYV | 17 |
| T366Y, L368A, P395R, | DGVEVHNAKTKPREEQYNSTYRVVSVLTVLHQDWLNGKEYKCKVSNKAL | |
| F405R, Y407M, K409A; | PAPIEKTISKAKGQPREPQVYTYPPSRDELTKNQVSLYCAVKGFYPSDI | |
| Kabat EU numbering) | AVEWESNGQPENNYKTTRPVLDSDGSFRLMSALTVDKSRWQQGNVFSCS | |
| | VMHEALHNHYTQKSLSLSPGK | |
| IgG1 mFc.23 (L351S, | APELLGGPSVFLFPPKPKDTLMISRTPEVTCVVVDVSHEDPEVKFNWYV | 18 |
| T366R, L368H, P395K, | DGVEVHNAKTKPREEQYNSTYRVVSVLTVLHQDWLNGKEYKCKVSNKAL | |
| F405E, Y407K, K409A; | PAPIEKTISKAKGQPREPQVYTSPPSRDELTKNQVSLRCHVKGFYPSDI | |
| Kabat EU numbering) | AVEWESNGQPENNYKTTKPVLDSDGSFELKSALTVDKSRWQQGNVESCS | |
| | VMHEALHNHYTQKSLSLSPGK | |
| IgG1 mFc.67 (L351K, | APELLGGPSVFLFPPKPKDTLMISRTPEVTCVVVDVSHEDPEVKFNWYV | 19 |
| T366S, P395V, F405R, | DGVEVHNAKTKPREEQYNSTYRVVSVLTVLHQDWLNGKEYKCKVSNKAL | |
| Y407A, K409Y; Kabat | PAPIEKTISKAKGQPREPQVYTKPPSRDELTKNQVSLSCLVKGFYPSDI | |
| EU numbering) | AVEWESNGQPENNYKTTVPVLDSDGSFRLASYLTVDKSRWQQGNVFSCS | |
| | VMHEALHNHYTQKSLSLSPGK | |

In some embodiments, the non-dimerizing Fc domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:14. In some embodiments, the non-dimerizing Fc domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:15. In some embodiments, the non-dimerizing Fc domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:16. In some embodiments, the non-dimerizing Fc domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:17. In some embodiments, the non-dimerizing Fc domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:18. In some embodiments, the non-dimerizing Fc domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:19.

In some embodiments, the IgG Fc domain further comprises, in addition to a CH2 and CH3 domain, an additional CH3 domain connected to the first CH3 domain via a linker (e.g., a linker as described in Section 6.5). An Fc domain comprising such a configuration (CH2-CH3-linker-CH3) is sometimes referred to herein as an “Fc1.5 domain” or simply “Fc1.5” for convenience. The linker between the first and second CH3 domains of an Fc1.5 domain is preferably of sufficient length and flexibility so as to permit dimerization of the first CH3 domain with the second CH3 domain. Thus, in some embodiments, the Fc1.5 domain comprises a linker of at least 5, at least 10, at least 15, or at least 20 amino acids in length connecting the first and second CH3 domains.

Exemplary sequences of Fc1.5 domains are provided in Table Y-3, below.

TABLE Y-3
Fc1.5 Sequences

| | | SEQ |
|---|---|---|
| Fc1.5 | Sequence | ID NO |
| Fc1.5 (IgG1; (G₄S)₈) | GGPSVFLFPPKPKDTLMISRTPEVTCVVVDVSHEDPEVKFNWYVDGVEV | 20 |
| | HNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWE | |
| | SNGQPENNYKTTPPVLDSDGSFFLYSKLTVDKSRWQQGNVFSCSVMHEA | |
| | LHNHYTQKSLSLSPGKGGGGSGGGGSGGGGSGGGGSGGGGSGGGGSGGG | |
| | GSGGGGSGQPREPQVYTLPPSRDELTKNQVSLTCLVKGFYPSDIAVEWE | |
| | SNGQPENNYKTTPPVLDSDGSFFLYSKLTVDKSRWQQGNVFSCSVMHEA | |
| | LHNHYTQKSLSLSPGK | |
| Fc1.5 (IgG1; (G₄S)₇) | GGPSVFLFPPKPKDTLMISRTPEVTCVVVDVSHEDPEVKFNWYVDGVEV | 21 |
| | HNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWE | |
| | SNGQPENNYKTTPPVLDSDGSFFLYSKLTVDKSRWQQGNVFSCSVMHEA | |
| | LHNHYTQKSLSLSPGKGGGGSGGGGSGGGGSGGGGSGGGGSGGGGSGGG | |
| | GSGQPREPQVYTLPPSRDELTKNQVSLTCLVKGFYPSDIAVEWESNGQP | |
| | ENNYKTTPPVLDSDGSFFLYSKLTVDKSRWQQGNVFSCSVMHEALHNHY | |
| | TQKSLSLSPGK | |
| Fc1.5 (IgG1; (G₄S)₆) | GGPSVFLFPPKPKDTLMISRTPEVTCVVVDVSHEDPEVKFNWYVDGVEV | 22 |
| | HNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWE | |
| | SNGQPENNYKTTPPVLDSDGSFFLYSKLTVDKSRWQQGNVFSCSVMHEA | |
| | LHNHYTQKSLSLSPGKGGGGSGGGGSGGGGSGGGGSGGGGSGGGGSGQP | |
| | REPQVYTLPPSRDELTKNQVSLTCLVKGFYPSDIAVEWESNGQPENNYK | |
| | TTPPVLDSDGSFFLYSKLTVDKSRWQQGNVFSCSVMHEALHNHYTQKSL | |
| | SLSPGK | |
| Fc1.5 (IgG1; (G₄S)₅) | GGPSVFLFPPKPKDTLMISRTPEVTCVVVDVSHEDPEVKFNWYVDGVEV | 23 |
| | HNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWE | |
| | SNGQPENNYKTTPPVLDSDGSFFLYSKLTVDKSRWQQGNVFSCSVMHEA | |
| | LHNHYTQKSLSLSPGKGGGGSGGGGSGGGGSGGGGSGGGGSGQPREPQV | |
| | YTLPPSRDELTKNQVSLTCLVKGFYPSDIAVEWESNGQPENNYKTTPPV | |
| | LDSDGSFFLYSKLTVDKSRWQQGNVFSCSVMHEALHNHYTQKSLSLSPG | |
| | K | |
| Fc1.5 (IgG1; (G₄S)₄) | GGPSVFLFPPKPKDTLMISRTPEVTCVVVDVSHEDPEVKFNWYVDGVEV | 24 |
| | HNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWE | |
| | SNGQPENNYKTTPPVLDSDGSFFLYSKLTVDKSRWQQGNVFSCSVMHEA | |
| | LHNHYTQKSLSLSPGKGGGGSGGGGSGGGGSGGGGSGQPREPQVYTLPP | |
| | SRDELTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGK | |
| Fc1.5 (IgG1; (G₄S)₃) | GGPSVFLFPPKPKDTLMISRTPEVTCVVVDVSHEDPEVKFNWYVDGVEV | 25 |
| | HNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWE | |
| | SNGQPENNYKTTPPVLDSDGSFFLYSKLTVDKSRWQQGNVFSCSVMHEA | |
| | LHNHYTQKSLSLSPGKGGGGSGGGGSGGGGSGQPREPQVYTLPPSRDEL | |
| | TKNQVSLTCLVKGFYPSDIAVEWESNGQPENNYKTTPPVLDSDGSFFLY | |
| | SKLTVDKSRWQQGNVFSCSVMHEALHNHYTQKSLSLSPGK | |
| Fc1.5 (IgG1; (G₄S)₂) | GGPSVFLFPPKPKDTLMISRTPEVTCVVVDVSHEDPEVKFNWYVDGVEV | 26 |
| | HNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWE | |
| | SNGQPENNYKTTPPVLDSDGSFFLYSKLTVDKSRWQQGNVFSCSVMHEA | |
| | LHNHYTQKSLSLSPGKGGGGSGGGGSGQPREPQVYTLPPSRDELTKNQV | |
| | SLTCLVKGFYPSDIAVEWESNGQPENNYKTTPPVLDSDGSFFLYSKLTV | |
| | DKSRWQQGNVFSCSVMHEALHNHYTQKSLSLSPGK | |
| Fc1.5 (IgG1; (G₄S)) | GGPSVFLFPPKPKDTLMISRTPEVTCVVVDVSHEDPEVKFNWYVDGVEV | 27 |
| | HNAKTKPREEQYNSTYRVVSVLTVLHQDWLNGKEYKCKVSNKALPAPIE | |
| | KTISKAKGQPREPQVYTLPPSRDELTKNQVSLTCLVKGFYPSDIAVEWE | |
| | SNGQPENNYKTTPPVLDSDGSFFLYSKLTVDKSRWQQGNVFSCSVMHEA | |
| | LHNHYTQKSLSLSPGKGGGGSGQPREPQVYTLPPSRDELTKNQVSLTCL | |
| | VKGFYPSDIAVEWESNGQPENNYKTTPPVLDSDGSFFLYSKLTVDKSRW | |
| | QQGNVFSCSVMHEALHNHYTQKSLSLSPGK | |

In some embodiments, the Fc1.5 domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:20. In some embodiments, the Fc1.5 domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:21. In some embodiments, the Fc1.5 domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:22. In some embodiments, the Fc1.5 domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:23. In some embodiments, the Fc1.5 domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:24. In some embodiments, the Fc1.5 domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:25. In some embodiments, the Fc1.5 domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:26. In some embodiments, the Fc1.5 domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:27.

Exemplary sequences of J chains linked to IgG Fc domains are provided in Table Y-4, below.

TABLE Y-4
IgG Fc-linked J chain

| | | SEQ |
|---|---|---|
| Molecule | Sequence | ID NO |
| IgG Fc domain-J | EPKSCDKTHTCPPCPAPELLGGPSVFLFPPKPKDTLMISRTPEVTCVVV | 28 |
| chain | DVSHEDPEVKFNWYVDGVEVHNAKTKPREEQYNSTYRVVSVLTVLHQDW | |
| | LNGKEYKCKVSNKALPAPIEKTISKAKGQPREPQVYTLPPSRDELTKNQ | |
| | VSLTCLVKGFYPSDIAVEWESNGQPENNYKTTPPVLDSDGSFFLYSKLT | |
| | VDKSRWQQGNVFSCSVMHEALHNHYTQKSLSLSPGKGGGGSQEDERIVL | |
| | VDNKCKCARITSRIIRSSEDPNEDIVERNIRIIVPLNNRENISDPTSPL | |
| | RTRFVYHLSDLCKKCDPTEVELDNQIVTATQSNICDEDSATETCYTYDR | |
| | NKCYTAVVPLVYGGETKMVETALTPDACYPD | |
| J chain-monomeric | QEDERIVLVDNKCKCARITSRIIRSSEDPNEDIVERNIRIIVPLNNREN | 29 |
| IgG Fc | ISDPTSPLRTRFVYHLSDLCKKCDPTEVELDNQIVTATQSNICDEDSAT | |
| | ETCYTYDRNKCYTAVVPLVYGGETKMVETALTPDACYPDGGGGSGGGGS | |
| | APELLGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKVSNKAL | |
| | PAPIEKTISKAKGQPREPQVYTSPPSRDELTKNQVSLRCHVKGFYPSDI | |
| | AVEWESNGQPENNYKTTKPVLDSDGSFFLYSKLTVDKSRWQQGNVFSCS | |
| | VMHEALHNHYTQKSLSLSPGK | |
| monomeric IgG Fc-J | APELLGGPSVFLFPPKPKDTLMISRTPEVTCVVVDVSHEDPEVKFNWYV | 30 |
| chain | DGVEVHNAKTKPREEQYNSTYRVVSVLTVLHQDWLNGKEYKCKVSNKAL | |
| | PAPIEKTISKAKGQPREPQVYTSPPSRDELTKNQVSLRCHVKGFYPSDI | |
| | AVEWESNGQPENNYKTTKPVLDSDGSFFLYSKLTVDKSRWQQGNVFSCS | |
| | VMHEALHNHYTQKSLSLSPGKGGGGSGGGGSQEDERIVLVDNKCKCARI | |
| | TSRIIRSSEDPNEDIVERNIRIIVPLNNRENISDPTSPLRTRFVYHLSD | |
| | LCKKCDPTEVELDNQIVTATQSNICDEDSATETCYTYDRNKCYTAVVPL | |
| | VYGGETKMVETALTPDACYPD | |
| J chain-Fc1.5 | QEDERIVLVDNKCKCARITSRIIRSSEDPNEDIVERNIRIIVPLNNREN | 31 |
| | ISDPTSPLRTRFVYHLSDLCKKCDPTEVELDNQIVTATQSNICDEDSAT | |
| | ETCYTYDRNKCYTAVVPLVYGGETKMVETALTPDACYPDGGGGSGGGGS | |
| | APELLGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKVSNKAL | |
| | PAPIEKTISKAKGQPREPQVYTLPPSRDELTKNQVSLTCLVKGFYPSDI | |
| | AVEWESNGQPENNYKTTPPVLDSDGSFFLYSKLTVDKSRWQQGNVFSCS | |
| | VMHEALHNHYTQKSLSLSPGKGGGGSGGGGSGGGGSGGGGSGQPREPQV | |
| | YTLPPSRDELTKNQVSLTCLVKGFYPSDIAVEWESNGQPENNYKTTPPV | |
| | LDSDGSFFLYSKLTVDKSRWQQGNVFSCSVMHEALHNHYTQKSLSLSPG | |
| | K | |
| Fc1.5-J chain | APELLGGPSVFLFPPKPKDTLMISRTPEVTCVVVDVSHEDPEVKFNWYV | 32 |
| | DGVEVHNAKTKPREEQYNSTYRVVSVLTVLHQDWLNGKEYKCKVSNKAL | |
| | PAPIEKTISKAKGQPREPQVYTLPPSRDELTKNQVSLTCLVKGFYPSDI | |
| | AVEWESNGQPENNYKTTPPVLDSDGSFFLYSKLTVDKSRWQQGNVFSCS | |
| | VMHEALHNHYTQKSLSLSPGKGGGGSGGGGSGGGGSGGGGSGQPREPQV | |
| | YTLPPSRDELTKNQVSLTCLVKGFYPSDIAVEWESNGQPENNYKTTPPV | |
| | LDSDGSFFLYSKLTVDKSRWQQGNVFSCSVMHEALHNHYTQKSLSLSPG | |
| | KGGGGSGGGGSQEDERIVLVDNKCKCARITSRIIRSSEDPNEDIVERNI | |
| | RIIVPLNNRENISDPTSPLRTRFVYHLSDLCKKCDPTEVELDNQIVTAT | |
| | QSNICDEDSATETCYTYDRNKCYTAVVPLVYGGETKMVETALTPDACYP | |
| | D | |

In some embodiments, the IgG Fc-linked J chain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:28. In some embodiments, the Fc1.5 domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:29. In some embodiments, the Fc1.5 domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:30. In some embodiments, the Fc1.5 domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:31. In some embodiments, the Fc1.5 domain comprises an amino acid sequence having at least about 90%, at least about 91%, at least about 92%, about at least 93%, at least about 94%, at least about 95%, at least about 96%, at least about 97%, at least about 98%, at least about 99%, or 100% sequence identity to SEQ ID NO:32.

6.4.3.1.1. IgG Fc Domains with Altered Effector Function

In some embodiments, the IgG Fc domain of IgG Fc-linked J chains of the disclosure comprises one or more amino acid substitutions that alter (e.g., reduce) binding to an Fc receptor and/or effector function.

In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

In one embodiment, the Fc domain (e.g., an Fc domain of an IgG Fc-linked J chain) comprises an amino acid substitution at a position selected from the group of E233, L234, L235, N297, P331 and P329 (numberings according to Kabat EU index). In a more specific embodiment, the Fc domain comprises an amino acid substitution at a position selected from the group of L234, L235 and P329 (numberings according to Kabat EU index). In some embodiments, the Fc domain comprises the amino acid substitutions L234A and L235A (numberings according to Kabat EU index). In one such embodiment, the Fc domain or region is an Igd Fc domain or region, particularly a human Igd Fc domain or region. In one embodiment, the Fc domain or the Fc region comprises an amino acid substitution at position P329. In a more specific embodiment, the amino acid substitution is P329A or P329G, particularly P329G (numberings according to Kabat EU index). In one embodiment, the Fc domain or the Fc region comprises an amino acid substitution at position P329 and a further amino acid substitution at a position selected from E233, L234, L235, N297 and P331 (numberings according to Kabat EU index). In a more specific embodiment, the further amino acid substitution is E233P, L234A, L235A, L235E, N297A, N297D or P331S. In particular embodiments, the Fc domain or the Fc region comprises amino acid substitutions at positions P329, L234 and L235 (numberings according to Kabat EU index). In more particular embodiments, the Fc domain comprises the amino acid mutations L234A, L235A and P329G (“P329G LALA”, “PGLALA” or “LALAPG”).

Typically, the same one or more amino acid substitution is present in each of the two Fc domains of an Fc region. Thus, in a particular embodiment, each Fc domain of the Fc region comprises the amino acid substitutions L234A, L235A and P329G (Kabat EU index numbering), i.e. in each of the first and the second Fc domains in the Fc region the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In one embodiment, the Fc domain is an IgG1 Fc domain, particularly a human IgG1 Fc domain. In some embodiments, the IgG1 Fc domain is a variant IgG1 comprising D265A, N297A mutations (EU numbering) to reduce effector function.

In another embodiment, the Fc domain is an IgG4 Fc domain with reduced binding to Fc receptors. Exemplary IgG4 Fc domains with reduced binding to Fc receptors may comprise an amino acid sequence selected from Table H below: In some embodiments, the Fc domain includes only the bolded portion of the sequences shown below:

TABLE H

| Fc Domain | Sequence |
|---|---|
| SEQ ID NO: 1 of | DKRVESKYGP PCPPCPAPPV AGPSVFLFPP KPKDTLMISR |
| WO2014/121087 | TPEVTCVVVD VSQEDPEVQF NWYVDGVEVH NAKTKPREEQ |
| | FNSTYRVVSV LTVLHQDWLN GKEYKCKVSN KGLPSSIEKT |
| | ISKAKGQPRE PQVYTLPPSQ EEMTKNQVSL TCLVKGFYPS |
| | DIAVEWESNG QPENNYKTTP PVLDSDGSFF LYSRLTVDKS |
| | RWQEGNVFSC SVMHEALHNH YTQKSLSLSL GK |
| SEQ ID NO: 2 of | DKKVEPKSCD KTHTCPPCPA PPVAGPSVFL FPPKPKDTLM |
| WO2014/121087 | ISRTPEVTCV VVDVSQEDPE VQFNWYVDGV EVHNAKTKPR |
| | EEQFNSTYRV VSVLTVLHQD WLNGKEYKCK VSNKGLPSSI |
| | EKTISKAKGQ PREPQVYTLP PSRDELTKNQ VSLTCLVKGF |
| | YPSDIAVEWE SNGQPENNYK TTPPVLDSDG SFFLYSKLTV |
| | DKSRWQQGNV FSCSVMHEAL HNHYTQKSLS LSPGK |
| SEQ ID NO: 30 of | ASTKGPSVFP LAPSSKSTSG GTAALGCLVK DYFPEPVTVS |
| WO2014/121087 | WNSGALTSGV HTFPAVLQSS GLYSLSSVVT VPSSSLGTQT |
| | YICNVNHKPS NTKVDKKVEP KSCDKTHTCP PCPAPPVAGP |
| | SVFLFPPKPK DTLMISRTPE VTCVVVDVSQ EDPEVQFNWY |
| | VDGVEVHNAK TKPREEQFNS TYRVVSVLTV LHQDWLNGKE |
| | YKCKVSNKGL PSSIEKTISK AKGQPREPQV YTLPPSRDEL |
| | TKNQVSLTCL VKGFYPSDIA VEWESNGQPE NNYKTTPPVL |
| | DSDGSFFLYS KLTVDKSRWQ QGNVFSCSVM HEALHNHYTQ |
| | KSLSLSPGK |
| SEQ ID NO: 31 of | ASTKGPSVFP LAPCSRSTSE STAALGCLVK DYFPEPVTVS |
| WO2014/121087 | WNSGALTSGV HTFPAVLQSS GLYSLSSVVT VPSSSLGTKT |
| | YTCNVDHKPS NTKVDKRVES KYGPPCPPCP APPVAGPSVF |
| | LFPPKPKDTL MISRTPEVTC VVVDVSQEDP EVQFNWYVDG |
| | VEVHNAKTKP REEQFNSTYR VVSVLTVLHQ DWLNGKEYKC |
| | KVSNKGLPSS IEKTISKAKG QPREPQVYTL PPSQEEMTKN |
| | QVSLTCLVKG FYPSDIAVEW ESNGQPENNY KTTPPVLDSD |
| | GSFFLYSRLT VDKSRWQEGN VFSCSVMHEA LHNHYTQKSL |
| | SLSLGK |
| SEQ ID NO: 37 of | ASTKGPSVFP LAPSSKSTSG GTAALGCLVK DYFPEPVTVS |
| WO2014/121087 | WNSGALTSGV HTFPAVLQSS GLYSLSSVVT VPSSSLGTQT |
| | YICNVNHKPS NTKVDKKVEP KSCDKTHTCP PCPAPPVAGP |
| | SVFLFPPKPK DTLMISRTPE VTCVVVDVSQ EDPEVQFNWY |
| | VDGVEVHNAK TKPREEQENS TYRVVSVLTV LHQDWLNGKE |
| | YKCKVSNKGL PSSIEKTISK AKGQPREPQV YTLPPSRDEL |
| | TKNQVSLTCL VKGFYPSDIA VEWESNGQPE NNYKTTPPVL |
| | DSDGSFFLYS KLTVDKSRWQ QGNVFSCSVM HEALHNRFTQ |
| | KSLSLSPGK |
| SEQ ID NO: 38 of | ASTKGPSVFP LAPCSRSTSE STAALGCLVK DYFPEPVTVS |
| WO2014/121087 | WNSGALTSGV HTFPAVLQSS GLYSLSSVVT VPSSSLGTKT |
| | YTCNVDHKPS NTKVDKRVES KYGPPCPPCP APPVAGPSVF |
| | LFPPKPKDTL MISRTPEVTC VVVDVSQEDP EVQFNWYVDG |
| | VEVHNAKTKP REEQFNSTYR VVSVLTVLHQ DWLNGKEYKC |
| | KVSNKGLPSS IEKTISKAKG QPREPQVYTL PPSQEEMTKN |
| | QVSLTCLVKG FYPSDIAVEW ESNGQPENNY KTTPPVLDSD |
| | GSFFLYSRLT VDKSRWQEGN VFSCSVMHEA LHNRFTQKSL |
| | SLSLGK |

In a particular embodiment, the IgG4 with reduced effector function comprises the bolded portion of the amino acid sequence of SEQ ID NO:31 of WO2014/121087, sometimes referred to herein as IgG4s or hIgG4s.

### 6.4.3.1.2. Hinge Domains

The IgG Fc-linked J chains of the disclosure can comprise an Fc domain comprising a hinge domain at its N-terminus. The hinge region can be a native or a modified hinge region. Hinge regions are typically found at the N-termini of Fc regions. The term “hinge domain”, unless the context dictates otherwise, refers to a naturally or non-naturally occurring hinge sequence that in the context of a single or monomeric polypeptide chain is a monomeric hinge domain and in the context of a dimeric polypeptide (e.g., an Fc region formed by the association of two Fc domains) can comprise two associated hinge sequences on separate polypeptide chains. Sometimes, the two associated hinge sequences are referred to as a “hinge region”. In certain embodiments of IgG Fc-linked J chains, additional iterations of hinge regions may be incorporated into the polypeptide sequence.

A native hinge region is the hinge region that would normally be found between Fab and Fc domains in a naturally occurring antibody. A modified hinge region is any hinge that differs in length and/or composition from the native hinge region. Such hinges can include hinge regions from other species, such as human, mouse, rat, rabbit, shark, pig, hamster, camel, llama, or goat hinge regions. Other modified hinge regions may comprise a complete hinge region derived from an antibody of a different class or subclass from that of the heavy chain Fc domain or Fc region. Alternatively, the modified hinge region may comprise part of a natural hinge or a repeating unit in which each unit in the repeat is derived from a natural hinge region. In a further alternative, the natural hinge region may be altered by converting one or more cysteine or other residues into neutral residues, such as serine or alanine, or by converting suitably placed residues into cysteine residues. By such means the number of cysteine residues in the hinge region may be increased or decreased. Other modified hinge regions may be entirely synthetic and may be designed to possess desired properties such as length, cysteine composition and flexibility.

A number of modified hinge regions have already been described for example, in U.S. Pat. No. 5,677,425, WO 99/15549, WO 2005/003170, WO 2005/003169, WO 2005/003170, WO 98/25971 and WO 2005/003171 and these are incorporated herein by reference.

In one embodiment, an IgG Fc-linked J chain comprises an Fc region in which one or both Fc domains possesses an intact hinge domain at its N-terminus.

In various embodiments, positions 233-236 within a hinge region may be G, G, G and unoccupied; G, G, unoccupied, and unoccupied; G, unoccupied, unoccupied, and unoccupied; or all unoccupied, with positions numbered by EU numbering.

In some embodiments, the IgG Fc-linked J chain comprises a modified hinge region that reduces binding affinity for an Fcγ receptor relative to a wild-type hinge region of the same isotype (e.g., human IgG1 or human IgG4).

In one embodiment, the IgG Fc-linked J chain comprises an Fc region in which each Fc domain possesses an intact hinge domain at its N-terminus, where each Fc domain and hinge domain is derived from IgG4 and each hinge domain comprises the modified sequence CPPC. The core hinge region of human IgG4 contains the sequence CPSC compared to IgG1 that contains the sequence CPPC. The serine residue present in the IgG4 sequence leads to increased flexibility in this region, and therefore a proportion of molecules form disulfide bonds within the same protein chain (an intrachain disulfide) rather than bridging to the other heavy chain in the IgG molecule to form the interchain disulfide. (Angel et al., 1993, Mol Immunol 30(1): 105-108). Changing the serine residue to a proline to give the same core sequence as IgG1 allows complete formation of inter-chain disulfides in the IgG4 hinge region, thus reducing heterogeneity in the purified product. This altered isotype is termed IgG4P.

### 6.4.3.1.2.1. Chimeric Hinge Sequences

The hinge domain can be a chimeric hinge domain. A “chimeric” hinge domain describes a hinge domain comprising a first region from a first type of IgG (e.g., IgG1, IgG2, IgG3, or IgG4), and a second region from a second, different type of IgG (e.g., IgG1, IgG2, IgG3, or IgG4).

For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region.

In particular embodiments, a chimeric hinge region comprises the amino acid sequence EPKSCDKTHTCPPCPAPPVA (previously disclosed as SEQ ID NO:8 of WO 2014/121087, which is incorporated by reference in its entirety herein) or ESKYGPPCPPCPAPPVA (previously disclosed as SEQ ID NO:9 of WO 2014/121087). Such chimeric hinge sequences can be suitably linked to an IgG4 CH2 region (for example by incorporation into an IgG4 Fc domain, for example a human or murine Fc domain, which can be further modified in the CH2 and/or CH3 domain to reduce effector function, for example as described in Section 6.4.3.1.1).

### 6.5. Linkers

In certain aspects, the present disclosure provides multivalent anti-spike protein binding molecules in which two or more components are connected to one another by a peptide linker. By way of example and not limitation, linkers can be used to connect a spike protein ABD to a multimerization moiety.

A peptide linker can range from 2 amino acids to 60 or more amino acids, and in certain aspects a peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length.

In particular aspects, a peptide linker is at least 5 amino acids, at least 6 amino acids or at least 7 amino acids in length and optionally is up to 30 amino acids, up to 40 amino acids, up to 50 amino acids or up to 60 amino acids in length.

In some embodiments of the foregoing, the linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

In some embodiments, the linker is a G4S linker. In some embodiments the linker comprises two consecutive G4S sequences, three consecutive G4S sequences, four consecutive G4S sequences, five consecutive G4S sequences, or six consecutive G4S sequences.

### 6.6. Nucleic Acids and Host Cells

In another aspect, the disclosure provides nucleic acids encoding multivalent anti-spike protein binding molecules of the disclosure. In some embodiments, the multivalent anti-spike protein binding molecules are encoded by a single nucleic acid. In other embodiments, the multivalent anti-spike protein binding molecules can be encoded by a plurality (e.g., two, three, four or more) nucleic acids.

A single nucleic acid can encode a multivalent anti-spike protein binding molecule that comprises a single polypeptide chain, a multivalent anti-spike protein binding molecule that comprises two or more polypeptide chains, or a portion of a multivalent anti-spike protein binding molecule that comprises more than two polypeptide chains (for example, a single nucleic acid can encode two polypeptide chains of a multivalent anti-spike protein binding molecule comprising three, four or more polypeptide chains, or three polypeptide chains of a multivalent anti-spike protein binding molecule comprising four or more polypeptide chains). For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements and separated by internal ribosome entry site (IRES) sequences allowing for translation into separate polypeptides.

In some embodiments, a multivalent anti-spike protein binding molecule comprising two or more polypeptide chains is encoded by two or more nucleic acids. The number of nucleic acids encoding a multivalent anti-spike protein binding molecule can be equal to or less than the number of polypeptide chains in the multivalent anti-spike protein binding molecule (for example, when two or more polypeptide chains are encoded by a single nucleic acid).

The nucleic acids of the disclosure can be DNA or RNA (e.g., mRNA).

In another aspect, the disclosure provides host cells and vectors containing the nucleic acids of the disclosure. The nucleic acids may be present in a single vector or separate vectors present in the same host cell or separate host cell, as described in more detail herein below.

### 6.6.1. Vectors

The disclosure provides vectors comprising nucleotide sequences encoding a multivalent anti-spike protein binding molecule or a component thereof described herein, for example one or two of the polypeptide chains of a multivalent anti-spike protein binding molecule. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes can be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed or introduced into the same cell by co-transformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid-based transfection, or other conventional techniques. Methods and conditions for culturing the resulting transfected cells and for recovering the expressed polypeptides are known to those skilled in the art and may be varied or optimized depending upon the specific expression vector and mammalian host cell employed, based upon the present description.

### 6.6.2. Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### 6.7. Pharmaceutical Compositions

The multivalent anti-spike protein binding molecules of the disclosure may be in the form of compositions comprising the multivalent anti-spike protein binding molecule and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the multivalent anti-spike protein binding molecules and, for therapeutic uses, the mode of administration.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intratumorally, intrathecally, topically, or locally. The most suitable route for administration in any given case will depend on the particular antibody, the subject, and the nature and severity of the disease and the physical condition of the subject. Typically, the pharmaceutical composition will be administered intravenously or subcutaneously.

Pharmaceutical compositions can be conveniently presented in unit dosage forms containing a predetermined amount of a multivalent anti-spike protein binding molecule of the disclosure per dose. The quantity of a multivalent anti-spike protein binding molecule included in a unit dose will depend on the disease being treated, as well as other factors as are well known in the art. Such unit dosages may be in the form of a lyophilized dry powder containing an amount of multivalent anti-spike protein binding molecule suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of multivalent anti-spike protein binding molecule suitable for a single administration.

The pharmaceutical compositions may also be supplied in bulk from containing quantities of multivalent anti-spike protein binding molecules suitable for multiple administrations.

Pharmaceutical compositions may be prepared for storage as lyophilized formulations or aqueous solutions by mixing a multivalent anti-spike protein binding molecule having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives should be nontoxic to the recipients at the dosages and concentrations employed.

Buffering agents help to maintain the pH in the range which approximates physiological conditions. They may be present at a wide variety of concentrations but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol, and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10 wt % per wt of multivalent anti-spike protein binding molecule.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188, etc.), and pluronic polyols. Non-ionic surfactants may be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

The multivalent anti-spike protein binding molecules of the disclosure can be formulated as pharmaceutical compositions comprising the multivalent anti-spike protein binding molecules, for example containing one or more pharmaceutically acceptable excipients or carriers. To prepare pharmaceutical or sterile compositions comprising the multivalent anti-spike protein binding molecules of the present disclosure, a multivalent anti-spike protein binding molecule preparation can be combined with one or more pharmaceutically acceptable excipient or carrier.

For example, formulations of multivalent anti-spike protein binding molecules can be prepared by mixing multivalent anti-spike protein binding molecules with physiologically acceptable carriers, excipients, or stabilizers in the form of, e.g., lyophilized powders, slurries, aqueous solutions, lotions, or suspensions (see, e.g., Hardman et al., 2001, Goodman and Gilman's The Pharmacological Basis of Therapeutics, McGraw-Hill, New York, N.Y.; Gennaro, 2000, Remington: The Science and Practice of Pharmacy, Lippincott, Williams, and Wilkins, New York, N.Y.; Avis, et al. (eds.), 1993, Pharmaceutical Dosage Forms: General Medications, Marcel Dekker, NY; Lieberman, et al. (eds.), 1990, Pharmaceutical Dosage Forms: Tablets, Marcel Dekker, NY; Lieberman, et al. (eds.), 1990, Pharmaceutical Dosage Forms: Disperse Systems, Marcel Dekker, NY; Weiner and Kotkoskie, 2000, Excipient Toxicity and Safety, Marcel Dekker, Inc., New York, N.Y.).

### 6.8. Therapeutic Indications and Methods of Treatment

The present disclosure provides methods for using and applications for multivalent anti-spike protein binding molecules of the disclosure.

In certain aspects, the disclosure provides a method of preventing or treating a disease or condition in which an interaction between a RBD of a coronavirus and cellular ACE2 is implicated. In some embodiments, the disease or condition is prevented or treated by neutralization of the spike protein. In various embodiments, neutralization of the spike proteins comprises (a) inhibiting the ability of spike protein to bind to a receptor such as ACE2, (b) inhibiting cleavage of the spike protein by a protease such as TMPRSS2, (c) inhibiting the spike protein from mediating (i) viral entry into a host cell or (ii) viral reproduction in a host cell, or (d) any combination of two, three, or all four of (a), (b), (c)(i), and (c)(ii).

Accordingly, in some embodiments, the multivalent anti-spike protein binding molecules and pharmaceutical compositions of the disclosure can be used to inhibit an interaction between a RBD of a coronavirus and cellular ACE2. In some embodiments, the disclosure provides methods of inhibiting the interaction between the RBD of SARS-CoV. In other embodiments, the disclosure provides methods of inhibiting the interaction between the RBD of SARS-CoV-2. Accordingly, in some embodiments, the disclosure provides methods of inhibiting an interaction between a RBD of a coronavirus and cellular ACE2, comprising administering to a subject in need thereof a multivalent anti-spike protein binding molecule pharmaceutical composition as described herein.

In some embodiments, the disclosure provides methods of administrating a multivalent anti-spike protein binding molecule pharmaceutical composition as described herein to a subject who has been exposed to a coronavirus but is not diagnosed with an infection. In other embodiments, the subject has been tested positive for a coronavirus but is asymptomatic. In yet other embodiments, the subject has been tested positive for a coronavirus and is presymptomatic. In further embodiments, the subject has been tested positive for a coronavirus and is symptomatic. In other embodiments, the subject has developed COVID-19 or another coronavirus-mediated disease or condition.

In some embodiments, the disclosure provides a method of reducing the severity of coronavirus infection, comprising administering to a subject in need thereof the multivalent anti-spike protein binding molecule pharmaceutical composition as described herein.

In some other embodiments, the disclosure provides a method of reducing the viral load of a coronavirus, comprising administering to a subject in need thereof the multivalent anti-spike protein binding molecule pharmaceutical composition as described herein.

In further embodiments, the disclosure provides a method of preventing disease progression in a subject with a coronavirus infection, comprising administering to a subject in need thereof the multivalent anti-spike protein binding molecule pharmaceutical composition as described herein.

In some embodiments, the disclosure provides a method of reducing the duration of a coronavirus infection, comprising administering to a subject in need thereof the multivalent anti-spike protein binding molecule pharmaceutical composition as described herein.

In other embodiments, the disclosure provides a method of reducing the risk of severe disease or death in a subject with a coronavirus infection, comprising administering to a subject in need thereof the multivalent anti-spike protein binding molecule pharmaceutical composition as described herein.

### 7. NUMBERED EMBODIMENTS

While various specific embodiments have been illustrated and described, it will be appreciated that various changes can be made without departing from the spirit and scope of the disclosure(s). The present disclosure is exemplified by the numbered embodiments set forth below. Unless otherwise specified, features of any of the concepts, aspects and/or embodiments described in the detailed description above are applicable mutatis mutandis to any of the following numbered embodiments.

In the numbered embodiments that follow, the multimerization moieties are preferably derived from a mammalian multimerization moiety (e.g., a human Fc domain), the antigen binding domains are preferably from a human or humanized antibody, and the subjects are preferably mammals (e.g., humans).

1. A multivalent anti-spike protein binding molecule comprising at least 5 anti-spike protein antigen-binding domains (ABDs) operably linked by one or more multimerization moieties.

2. The multivalent anti-spike protein binding molecule of embodiment 1, which comprises at least 10 anti-spike protein ABDs.

3. The multivalent anti-spike protein binding molecule of embodiment 1 or embodiment 2, which is decavalent or dodecavalent.

4. The multivalent anti-spike protein binding molecule of any one of embodiments 1 to 3, wherein the antigen-binding domains (ABDs) are human or humanized.

5. The multivalent anti-spike protein binding molecule of any one of embodiments 1 to 4, wherein the antigen-binding domains (ABDs) are Fabs.

6. The multivalent anti-spike protein binding molecule of any one of embodiments 1 to 5, wherein one or more (or all) ABDs comprise CDR sequences set forth in any one of Tables 1-3.

7. The multivalent anti-spike protein binding molecule of any one of embodiments 1 to 5, wherein one or more (or all) ABDs comprise CDR sequences set forth in any one of Tables 1-4.

8. The multivalent anti-spike protein binding molecule of any one of embodiments 1 to 5, wherein one or more (or all) ABDs comprise the CDR-H1, CDR-H2, CDR-H3, CDR-L1, CDR-L2, and CDR-L3 sequences of an antibody set forth in Table 1.

9. The multivalent anti-spike protein binding molecule of any one of embodiments 1 to 5, wherein one or more (or all) ABDs comprise the CDR-H1, CDR-H2, CDR-H3, CDR-L1, CDR-L2, and CDR-L3 sequences of an antibody set forth in Table 2.

10. The multivalent anti-spike protein binding molecule of any one of embodiments 1 to 5, wherein one or more (or all) ABDs comprise the CDR-H1, CDR-H2, CDR-H3, CDR-L1, CDR-L2, and CDR-L3 sequences of an antibody set forth in Table 3.

11. The multivalent anti-spike protein binding molecule of any one of embodiments 1 to 5, wherein one or more (or all) ABDs comprise the CDR-H1, CDR-H2, CDR-H3, CDR-L1, CDR-L2, and CDR-L3 sequences of an antibody set forth in Table 4.

12. The multivalent anti-spike protein binding molecule of any one of embodiments 1 to 5, wherein one or more (or all) ABDs comprise:
(a) a VH comprising CDR-H1, CDR-H2, and CDR-H3 having the amino acid sequences of SEQ ID NOs: 579, 580, and 581, respectively; and
(b) a VL comprising CDR-L1, CDR-L2, and CDR-L3 having the amino acid sequences of SEQ ID NOs: 398, 372, and 583, respectively.

13. The multivalent anti-spike protein binding molecule of any one of embodiments 1 to 5, wherein one or more (or all) ABDs comprise:
(a) a VH comprising CDR-H1, CDR-H2, and CDR-H3 having the amino acid sequences of SEQ ID NOs: 507, 508, and 509, respectively; and
(b) a VL comprising CDR-L1, CDR-L2, and CDR-L3 having the amino acid sequences of SEQ ID NOs: 511, 407, and 512, respectively.

14. The multivalent anti-spike protein binding molecule of any one of embodiments 1 to 5, wherein one or more (or all) ABDs comprise:
(a) a VH comprising CDR-H1, CDR-H2, and CDR-H3 having the amino acid sequences of SEQ ID NOs: 450, 451, and 452, respectively; and
(b) a VL comprising CDR-L1, CDR-L2, and CDR-L3 having the amino acid sequences of SEQ ID NOs: 454, 415, and 455, respectively.

15. The multivalent anti-spike protein binding molecule of any one of embodiments 1 to 5, wherein one or more (or all) ABDs comprise VH and VL sequences set forth in any one of Tables 1-3.

16. The multivalent anti-spike protein binding molecule of any one of embodiments 1 to 5, wherein one or more (or all) ABDs comprise VH and VL sequences set forth in any one of Tables 1-4.

17. The multivalent anti-spike protein binding molecule of any one of embodiments 1 to 5, wherein one or more (or all) ABDs comprise VH and VL sequences of an antibody set forth in Table 1.

18. The multivalent anti-spike protein binding molecule of any one of embodiments 1 to 5, wherein one or more (or all) ABDs comprise VH and VL sequences of an antibody set forth in Table 2.

19. The multivalent anti-spike protein binding molecule of any one of embodiments 1 to 5, wherein one or more (or all) ABDs comprise VH and VL sequences of an antibody set forth in Table 3.

20. The multivalent anti-spike protein binding molecule of any one of embodiments 1 to 5, wherein one or more (or all) ABDs comprise VH and VL sequences of an antibody set forth in Table 4.

21. The multivalent anti-spike protein binding molecule of any one of embodiments 1 to 5, wherein one or more (or all) ABDs comprise (a) a VH having at least 90%, at least 95%, at least 96%, at least 97%, at least 98%, at least 99%, or 100% sequence identity to the amino acid sequence of SEQ ID NO:578 and (b) a VL having at least 90%, at least 95%, at least 96%, at least 97%, at least 98%, at least 99%, or 100% sequence identity to the amino acid sequence of SEQ ID NO:582.

22. The multivalent anti-spike protein binding molecule of any one of embodiments 1 to 5, wherein one or more (or all) ABDs comprise (a) a VH comprising the amino acid sequence of SEQ ID NO:578 and (b) a VL comprising the amino acid sequence of SEQ ID NO:582.

23. The multivalent anti-spike protein binding molecule of any one of embodiments 1 to 5, wherein one or more (or all) ABDs comprise (a) a VH having at least 90%, at least 95%, at least 96%, at least 97%, at least 98%, at least 99%, or 100% sequence identity to the amino acid sequence of SEQ ID NO:506 and (b) a VL having at least 90%, at least 95%, at least 96%, at least 97%, at least 98%, at least 99%, or 100% sequence identity to the amino acid sequence of SEQ ID NO:510.

24. The multivalent anti-spike protein binding molecule of any one of embodiments 1 to 5, wherein one or more (or all) ABDs comprise (a) a VH comprising the amino acid sequence of SEQ ID NO:506 and (b) a VL comprising the amino acid sequence of SEQ ID NO:510.

25. The multivalent anti-spike protein binding molecule of any one of embodiments 1 to 5, wherein one or more (or all) ABDs comprise (a) a VH having at least 90%, at least 95%, at least 96%, at least 97%, at least 98%, at least 99%, or 100% sequence identity to the amino acid sequence of SEQ ID NO:449 and (b) a VL having at least 90%, at least 95%, at least 96%, at least 97%, at least 98%, at least 99%, or 100% sequence identity to the amino acid sequence of SEQ ID NO:453.

26. The multivalent anti-spike protein binding molecule of any one of embodiments 1 to 5, wherein one or more (or all) ABDs comprise (a) a VH comprising the amino acid sequence of SEQ ID NO:449 and (b) a VL comprising the amino acid sequence of SEQ ID NO:453.

27. The multivalent anti-spike protein binding molecule of any one of embodiments 1 to 26, wherein one or more (or all) ABDs are neutralizing.

28. The multivalent anti-spike protein binding molecule of any one of embodiments 1 to 26, which is capable of neutralizing the SARS-CoV-2 variant BA. 1.

29. The multivalent anti-spike protein binding molecule of any one of embodiments 1 to 28, which is capable of neutralizing the SARS-CoV-2 variant BA.2.

30. The multivalent anti-spike protein binding molecule of any one of embodiments 1 to 29, which is monospecific.

31. The multivalent anti-spike protein binding molecule of any one of embodiments 1 to 30, in which the antigen-binding domains (ABDs) are all the same.

32. The multivalent anti-spike protein binding molecule of any one of embodiments 1 to 29, which is multispecific.

33. The multivalent anti-spike protein binding molecule of embodiment 32, which is bispecific.

34. The multivalent anti-spike protein binding molecule of embodiment 32 or embodiment 33, which comprises two types of ABDs.

35. The multivalent anti-spike protein binding molecule of any one of embodiments 1 to 34, wherein the one or more multimerization moieties comprise an Fc domain.

36. The multivalent anti-spike protein binding molecule of embodiment 34, wherein both types of ABDs bind to spike protein.

37. The multivalent anti-spike protein binding molecule of embodiment 34, wherein one type of ABD binds to spike protein and the other type of ABD binds to a different target.

38. The multivalent anti-spike protein binding molecule of embodiment 35, wherein the Fc domain is an IgM Fc domain.

39. The multivalent anti-spike protein binding molecule of embodiment 38, wherein the Fc domain comprises a Cμ3 domain and a Cμ4 domain.

40. The multivalent anti-spike protein binding molecule of embodiment 38 or embodiment 39, wherein the Fc domain comprises a Cμ2 domain.

41. The multivalent anti-spike protein binding molecule of any one of embodiments 38 to 40, which is a pentamer.

42. The multivalent anti-spike protein binding molecule of embodiment 41, which is a pentamer of five dimers, each dimer comprising two polypeptides, each polypeptide comprising an anti-spike protein ABD and an IgM Fc domain.

43. The multivalent anti-spike protein binding molecule of embodiment 41 or embodiment 42, which is a homopentamer.

44. The multivalent anti-spike protein binding molecule of any one of embodiments 41 to 43, in which a portion or all Cμ3 and/or Cμ4 domains are disulfide linked.

45. The multivalent anti-spike protein binding molecule of any one of embodiments 41 to 44, which comprises a J chain.

46. The multivalent anti-spike protein binding molecule of embodiment 45, wherein the J chain is operably linked to an IgG Fc domain.

47. The multivalent anti-spike protein binding molecule of embodiment 46, wherein the IgG Fc domain is N-terminal to the J chain.

48. The multivalent anti-spike protein binding molecule of embodiment 46, wherein the IgG Fc domain is C-terminal to the J chain.

49. The multivalent anti-spike protein binding molecule of any one of embodiments 46 to 48, wherein the IgG Fc domain and the J chain are connected via a linker.

50. The multivalent anti-spike protein binding molecule of embodiment 49, wherein the linker is or comprises the amino acid sequence G4S.

51. The multivalent anti-spike protein binding molecule of any one of embodiments 46 to 50, wherein the IgG Fc domain is an IgG1, IgG2, IgG3, or IgG4 Fc domain.

52. The multivalent anti-spike protein binding molecule of any one of embodiments 46 to 50, wherein the IgG Fc domain is an IgG1 domain.

53. The multivalent anti-spike protein binding molecule of any one of embodiments 46 to 50, wherein the IgG Fc domain is an IgG4 domain.

54. The multivalent anti-spike protein binding molecule of embodiment 33 or embodiment 53, wherein the Fc domain comprises a hinge at its N-terminus.

55. The multivalent anti-spike protein binding molecule of embodiment 54, wherein the hinge is a chimeric hinge.

56. The multivalent anti-spike protein binding molecule of any one of embodiments 46 to 55, wherein the IgG Fc domain is a non-dimerizing Fc domain.

57. The multivalent anti-spike protein binding molecule of embodiment 56, wherein the non-dimerizing Fc domain comprises the amino acid sequence of any one of SEQ ID NOs: 14-19.

58. The multivalent anti-spike protein binding molecule of any one of embodiments 46 to 51, wherein the IgG Fc domain is an Fc 1.5 domain.

59. The multivalent anti-spike protein binding molecule of embodiment 58, wherein the Fc 1.5 domain comprises the amino acid sequence of any one of SEQ ID NOs:20-27.

60. The multivalent anti-spike protein binding molecule of any one of embodiments 46 to 59, wherein the J chain operably linked to the IgG Fc domain comprises the amino acid sequence of any one of SEQ ID NOs:28-32.

61. A multivalent anti-spike protein binding molecule, which is optionally a multivalent anti-spike protein binding molecule according to any one of embodiments 1 to 60, which has the configuration depicted in FIG. 1A.

62. The multivalent anti-spike protein binding molecule of any one of embodiments 38 to 40, which is a hexamer.

63. The multivalent anti-spike protein binding molecule of embodiment 62, which is a hexamer of six dimers, each dimer comprising two polypeptides, each polypeptide comprising an anti-spike protein ABD and an IgM Fc domain.

64. The multivalent anti-spike protein binding molecule of embodiment 62 or embodiment 63 which is a homohexamer.

65. The multivalent anti-spike protein binding molecule of any one of embodiments 62 to 64, in which a portion or all Cμ3 and/or Cμ4 domains are disulfide linked.

66. The multivalent anti-spike protein binding molecule of any one of embodiments 62 to 65, which lacks a J chain.

67. A multivalent anti-spike protein binding molecule, which is optionally a multivalent anti-spike protein binding molecule according to any one of embodiments 1 to 40 and 62 to 66, which has the configuration depicted in FIG. 1B.

68. A multivalent anti-spike protein binding molecule, which is optionally a multivalent anti-spike protein binding molecule according to any one of embodiments 1 to 61, which has the configuration depicted in FIG. 1C.

69. A multivalent anti-spike protein binding molecule, which is optionally a multivalent anti-spike protein binding molecule according to any one of embodiments 1 to 61, which has the configuration depicted in FIG. 1D.

70. A multivalent anti-spike protein binding molecule, which is optionally a multivalent anti-spike protein binding molecule according to any one of embodiments 1 to 61, which has the configuration depicted in FIG. 1E.

71. A multivalent anti-spike protein binding molecule comprising at least 5 means for binding spike protein operably linked by one or more multimerization moieties.

72. The multivalent anti-spike protein binding molecule of embodiment 71, which comprises at least 10 means for binding spike protein.

73. The multivalent anti-spike protein binding molecule of embodiment 71 or 72, which is decavalent or dodecavalent for the means for binding spike protein.

74. The multivalent anti-spike protein binding molecule of any one of embodiments 71 to 73, which comprises at least five Fabs, each comprising means for binding spike protein.

75. The multivalent anti-spike protein binding molecule of any one of embodiments 71 to 74, which is monospecific.

76. The multivalent anti-spike protein binding molecule of embodiment 75, wherein the at least 5 means for binding spike protein are the same.

77. The multivalent anti-spike protein binding molecule of any one of embodiments 71 to 73, which is multispecific.

78. The multivalent anti-spike protein binding molecule of embodiment 77, which is bispecific.

79. The multivalent anti-spike protein binding molecule of any one of embodiments 71 to 78, wherein the one or more multimerization moieties comprise an Fc domain.

80. The multivalent anti-spike protein binding molecule of embodiment 79, wherein the Fc domain is an IgM Fc domain.

81. The multivalent anti-spike protein binding molecule of embodiment 80, wherein the Fc domain comprises a Cμ3 domain and a Cμ4 domain.

82. The multivalent anti-spike protein binding molecule of embodiment 80 or embodiment 81, wherein the Fc domain comprises a Cμ2 domain.

83. The multivalent anti-spike protein binding molecule of any one of embodiments 80 to 82, which is a pentamer.

84. The multivalent anti-spike protein binding molecule of embodiment 83, which is a pentamer of five dimers, each dimer comprising two polypeptides, each polypeptide comprising means for binding spike protein and an IgM Fc domain.

85. The multivalent anti-spike protein binding molecule of embodiment 83 or embodiment 84, which is a homopentamer.

86. The multivalent anti-spike protein binding molecule of any one of embodiments 83 to 85, in which a portion or all Cμ3 and/or Cμ4 domains are disulfide linked.

87. The multivalent anti-spike protein binding molecule of any one of embodiments 83 to 86, which comprises a J chain.

88. The multivalent anti-spike protein binding molecule of embodiment 87, wherein the J chain is operably linked to an IgG Fc domain.

89. The multivalent anti-spike protein binding molecule of embodiment 87, wherein the IgG Fc domain is N-terminal to the J chain.

90. The multivalent anti-spike protein binding molecule of embodiment 87, wherein the IgG Fc domain is C-terminal to the J chain.

91. The multivalent anti-spike protein binding molecule of any one of embodiments 87 to 90, wherein the IgG Fc domain and the J chain are connected via a linker.

92. The multivalent anti-spike protein binding molecule of embodiment 91, wherein the linker is or comprises the amino acid sequence G4S.

93. The multivalent anti-spike protein binding molecule of any one of embodiments 88 to 92, wherein the IgG Fc domain is an IgG1, IgG2, IgG3, or IgG4 Fc domain.

94. The multivalent anti-spike protein binding molecule of any one of embodiments 88 to 93, wherein the IgG Fc domain is an IgG1 domain.

95. The multivalent anti-spike protein binding molecule of any one of embodiments 88 to 94, wherein the IgG Fc domain is an IgG4 domain.

96. The multivalent anti-spike protein binding molecule of embodiment 33 or embodiment 95, wherein the Fc domain comprises a hinge at its N-terminus.

97. The multivalent anti-spike protein binding molecule of embodiment 96, wherein the hinge is a chimeric hinge.

98. The multivalent anti-spike protein binding molecule of any one of embodiments 88 to 97, wherein the IgG Fc domain is a non-dimerizing Fc domain.

99. The multivalent anti-spike protein binding molecule of embodiment 98, wherein the non-dimerizing Fc domain comprises the amino acid sequence of any one of SEQ ID NOs: 14-19.

100. The multivalent anti-spike protein binding molecule of any one of embodiments 88 to 97, wherein the IgG Fc domain is an Fc 1.5 domain.

101. The multivalent anti-spike protein binding molecule of embodiment 100, wherein the Fc 1.5 domain comprises the amino acid sequence of any one of SEQ ID NOs:20-27.

102. The multivalent anti-spike protein binding molecule of any one of embodiments 88 to 101, wherein the J chain operably linked to the IgG Fc domain comprises the amino acid sequence of any one of SEQ ID NOs:28-32.

103. A nucleic acid or plurality of nucleic acids encoding the multivalent anti-spike protein binding molecule of any one of embodiments 1 to 102.

104. A host cell engineered to express the multivalent anti-spike protein binding molecule of any one of embodiments 1 to 102 or the nucleic acid(s) of embodiment 103.

105. A method of producing the multivalent anti-spike protein binding molecule of any one of embodiments 1 to 102, comprising culturing the host cell of embodiment 104 and recovering the multivalent anti-spike protein binding molecule expressed thereby.

106. A pharmaceutical composition comprising the multivalent anti-spike protein binding molecule of any one of embodiments 1 to 102 and an excipient.

107. A method of treating a coronavirus disease, comprising administering to a subject in need thereof the multivalent anti-spike protein binding molecule of any one of embodiments 1 to 102 or the pharmaceutical composition of embodiment 106.

108. A method of inhibiting an interaction between a RBD of a coronavirus and cellular ACE2, comprising administering to a subject in need thereof the multivalent anti-spike protein binding molecule of any one of embodiments 1 to 102 or the pharmaceutical composition of embodiment 106.

109. A method of neutralizing a coronavirus spike protein in vivo, comprising administering to a subject in need thereof the multivalent anti-spike protein binding molecule of any one of embodiments 1 to 102 or the pharmaceutical composition of embodiment 106.

110. A method of inhibiting protease-mediated cleavage (e.g., TMPRSS2-mediated cleavage) of a coronavirus spike protein in vivo, comprising administering to a subject in need thereof the multivalent anti-spike protein binding molecule of any one of embodiments 1 to 102 or the pharmaceutical composition of embodiment 106.

111. A method of inhibiting viral entry of a coronavirus into a host cell in a subject, comprising administering to a subject in need thereof the multivalent anti-spike protein binding molecule of any one of embodiments 1 to 102 or the pharmaceutical composition of embodiment 106.

112. A method of inhibiting reproduction of a coronavirus spike protein in a host cell in a subject, comprising administering to a subject in need thereof the multivalent anti-spike protein binding molecule of any one of embodiments 1 to 102 or the pharmaceutical composition of embodiment 106.

113. A method reducing the severity of coronavirus infection, comprising administering to a subject in need thereof the multivalent anti-spike protein binding molecule of any one of embodiments 1 to 102 or the pharmaceutical composition of embodiment 106.

114. A method of reducing the viral load of a coronavirus, comprising administering to a subject in need thereof the multivalent anti-spike protein binding molecule of any one of embodiments 1 to 102 or the pharmaceutical composition of embodiment 106.

115. A method of preventing disease progression in a subject with a coronavirus infection, comprising administering to a subject in need thereof the multivalent anti-spike protein binding molecule of any one of embodiments 1 to 102 or the pharmaceutical composition of embodiment 106.

116. A method of reducing the duration of a coronavirus infection, comprising administering to a subject in need thereof the multivalent anti-spike protein binding molecule of any one of embodiments 1 to 102 or the pharmaceutical composition of embodiment 106.

117. A method of reducing the risk of severe disease or death in a subject with a coronavirus infection, comprising administering to a subject in need thereof the multivalent anti-spike protein binding molecule of any one of embodiments 1 to 102 or the pharmaceutical composition of embodiment 106.

118. The method of any one of embodiments 107 to 117, wherein the coronavirus is SARS-CoV.

119. The method of any one of embodiments 107 to 117, wherein the coronavirus is SARS-CoV-2.

### 8. EXAMPLES

### 8.1. Materials and Methods

### 8.1.1. Construction and Production of Anti-SARS-CoV-2 IgM Constructs

IgM heavy chain constructs were designed as DNA fragments with the following components from 5′ to 3′ end: an mROR1 signal sequence, a heavy chain variable region, and the constant region of IgM heavy chain (Uniprot ID: P01871). All IgM antibodies were expressed in FreeStyle™ 293-F cells (ThermoFisher) by transient transfection, following the manufacturer's protocol, whereby 250 ml of cells were transfected for each IgM antibody constructs with three chains (H, L, J) at a ratio of 1:1:1.

Antibodies were purified from the supernatant using POROS CaptureSelect IgM Affinity Matrix (ThermoFisher). First, the columns were equilibrated with 5 column volumes (CV) of PBS. Next, sterile filtered supernatant containing fusion proteins was loaded over the pre-equilibrated column at a flow rate of ˜2.0 mL/min. Any non-specifically bound materials were washed out of the column using 50 mM Tris-HCl, 500 mM NaCl, pH7.5 at a flow rate of 2.0 mL/min for 5 CV. The affinity-bound fusion protein was eluted from the column using Pierce™ IgG Elution Buffer (pH 2.8, Thermo Fisher). After elution, the proteins were neutralized using 1/10 v/v 1M Tris-HCl, pH8.0, The elution fraction material was further polished to increase the purity of the species of interest by SEC. Hence, a Superose 6 10/300GL column (Cytiva) was employed at a flow rate of 0.75 mL/min, in 1×DPBS, pH7.1 running buffer. Fractions of interest were pooled and concentrated. Each fraction pool was analyzed by UV-Vis to determine the protein concentration. Each fraction pool was further analyzed by SE-UPLC to determine the relative purity of the species of interest. The proteins isolated from each fraction pool were analyzed under denaturing conditions using SDS-PAGE. Furthermore, the samples were also run for 1 hour at 200 V constant on 4-20% Tris-Glycine gels that were loaded with 2 μg of sample per well.

### 8.1.2. SARS-CoV-2 Pseudovirus/Variant Neutralization Assay

Vero cells were cultured in DMEM high glucose medium with sodium pyruvate and without glutamine, supplemented with 10% heat-inactivated FBS and Penicillin/Streptomycin/L-glutamine (Complete DMEM) and seeded at 20,000 cells/well in 96-well black/clear bottom cell culture plates. On the day of the assay, the antibodies were diluted to 2×assay concentration and serially diluted 3-fold, for a total of 11 concentrations (e.g., 40 nM to 677.4 fM for IgG controls. For IgM molecules, concentrations used were 13.3 nM to 225.8 fM for experiment in Table 7 and 1.3 nM to 22.5 fM for experiments in Tables 8 and 9). All dilutions were performed using infection media consisting of DMEM high glucose medium without sodium pyruvate/with glutamine that was supplemented with Sodium Pyruvate, 0.2% IgG-free BSA, and Gentamicin.

The pVSV-Luc-SARS-CoV-2-S pseudoviruses used herein were non-replicating VSV-DG, that expressed a dual GFP/firefly luciferase reporter in place of its native glycoprotein, and pseudotyped with SARS-CoV-2 Spike. The SARS-CoV-2 pseudoviruses or variants were diluted 1:4 in infection media, then combined 1:1 with antibody dilutions for a final pseudovirus/variant dilution of 1:8 and final test article concentrations of 20 nM to 338.7 fM for IgG controls, with concentrations from 6.7 nM to 112.9 fM for IgM molecules in Table 7 and 2.0 nM to 33.8 fM for IgM molecules in Tables 8 and 9. Combined antibodies and pseudoviruses/variants were incubated at room temperature for 30 minutes. Next, the culture media were removed from the cells and the combined antibodies and 100 uL/well pseudoviruses/variants were added to the wells in duplicates, which were then incubated at 37° C., 5% CO₂ for 24 hours. At 24 hours post-infection, media were removed from the wells, and the cells were lysed using 100 μL/well Glo-Lysis buffer (Promega). Immediately before reading luminescence on the Spectramax i3X plate reader, 100 uL prepared Bright-Glo substrate (Promega) was added to the lysates. The results were exported to Microsoft Excel, where % neutralization was calculated with the following equation: % Neutralization=((1−(well value−medium control)/(virus control−medium control))×100% Neutralization is then plotted in GraphPad Prism and analyzed using nonlinear regression: log(inhibitor) vs. response—Variable slope (four parameter) to calculate IC50 values.

### 8.2. Example 1: Production of Anti-SARS-CoV-2 IgM Constructs

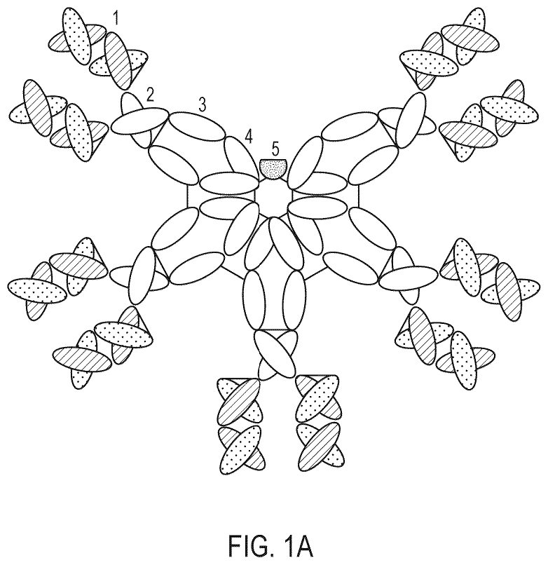

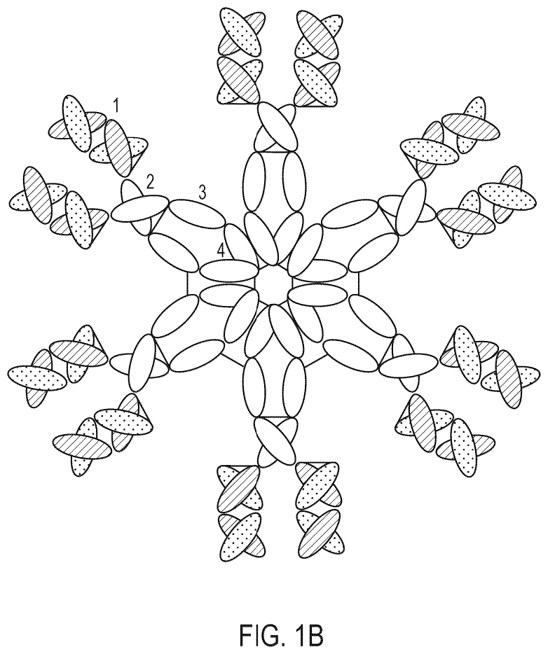

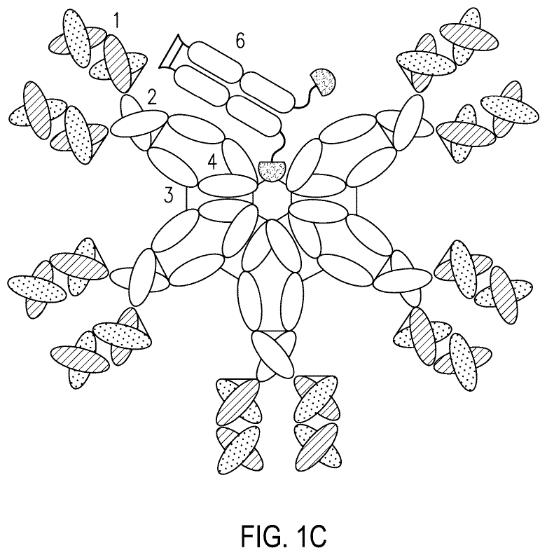

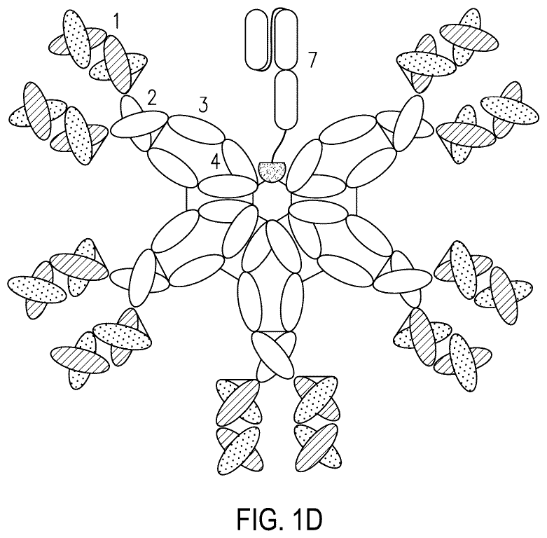

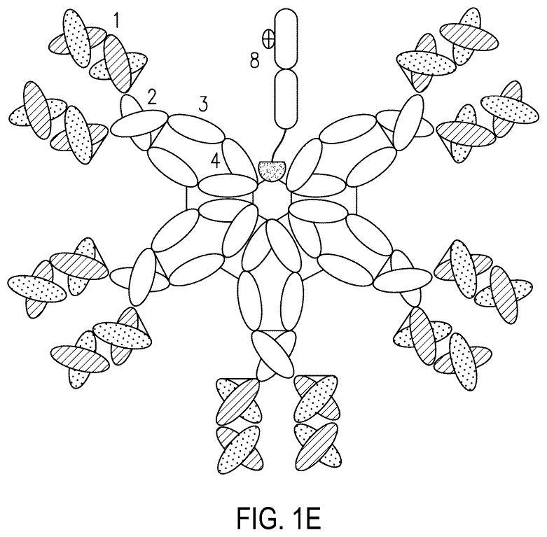

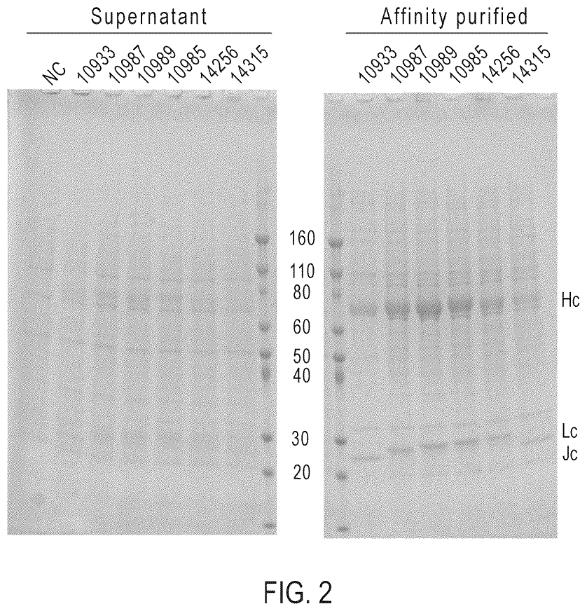

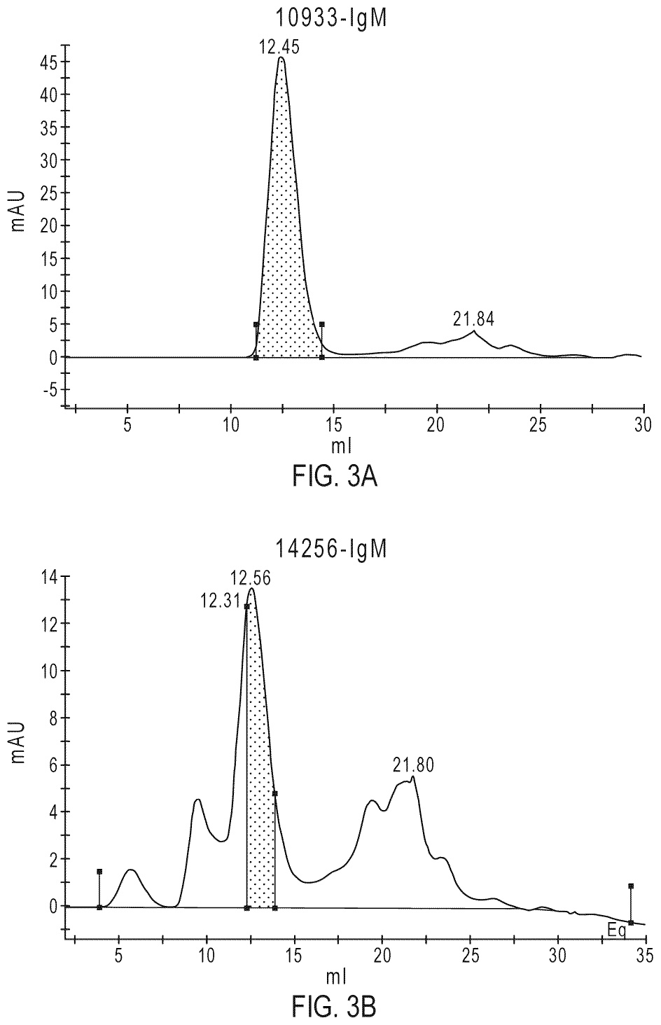

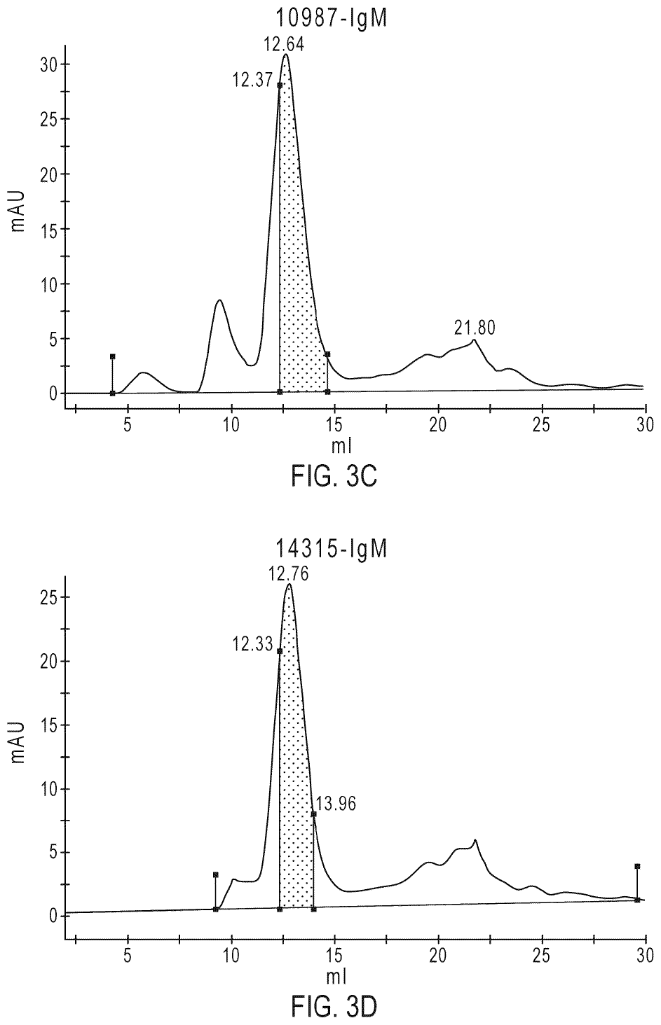

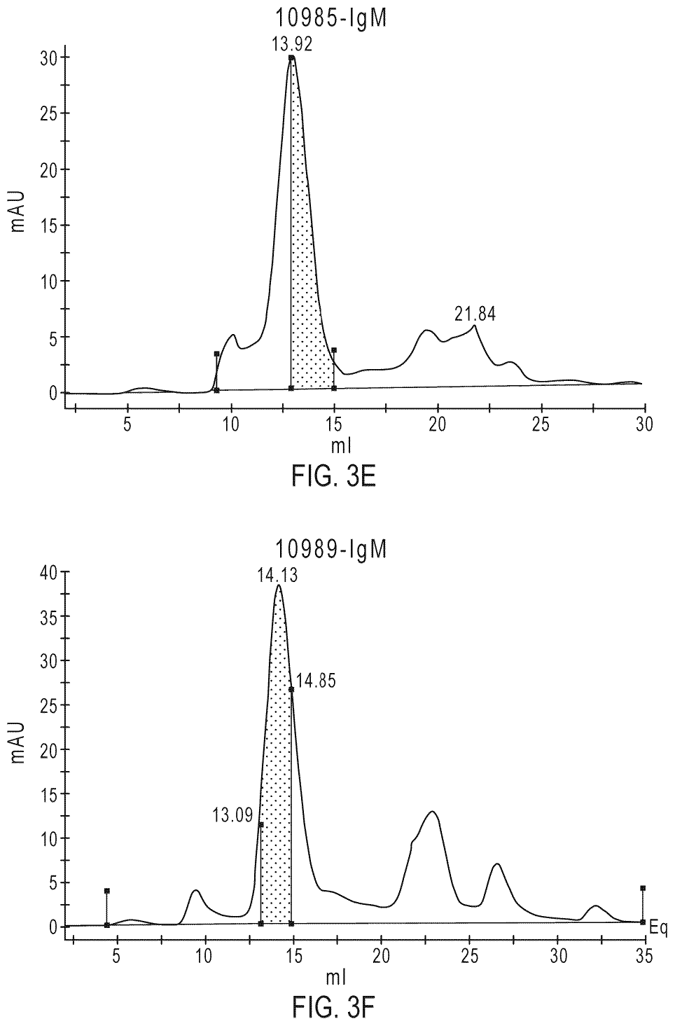

With different Fab moieties against SARS-Cov-2 S protein, a total of seven anti-SARS-CoV-2 IgMs (REGN10933, 10985, 10987, 10989, 14256, 14315, 14287; comprising VH and VL domains from mAb10933, mAb10985, mAb10987, mAb10989, mAb14256, mAb14315, and mAb14287, as set forth in Tables 3 and 4, respectively) were generated and purified as described in Section 8.1.1. Affinity purification was associated with thicker bands on non-reducing SDS-PAGE gels that corresponded to the expected product sizes (FIG. 2). Moreover, the SEC purification profiles of the six anti-SARS-CoV2 IgM constructs displayed discrete main peaks with varying levels of high molecular weight species (FIG. 3).

### 8.3. Example 2: Neutralization Activity of the RBD-targeted Anti-SARS-CoV-2 IgMs

A set of virus neutralization assays were conducted as described in Section 8.1.2. to compare the percent neutralization activities against SARS-CoV2 pseudovirus D614G and Omicron variants BA. 1 and BA.2 of all six anti-SARS-CoV-2 IgMs characterized in example 1, in comparison to REGEN-COV (REGN10987/REGN10933).

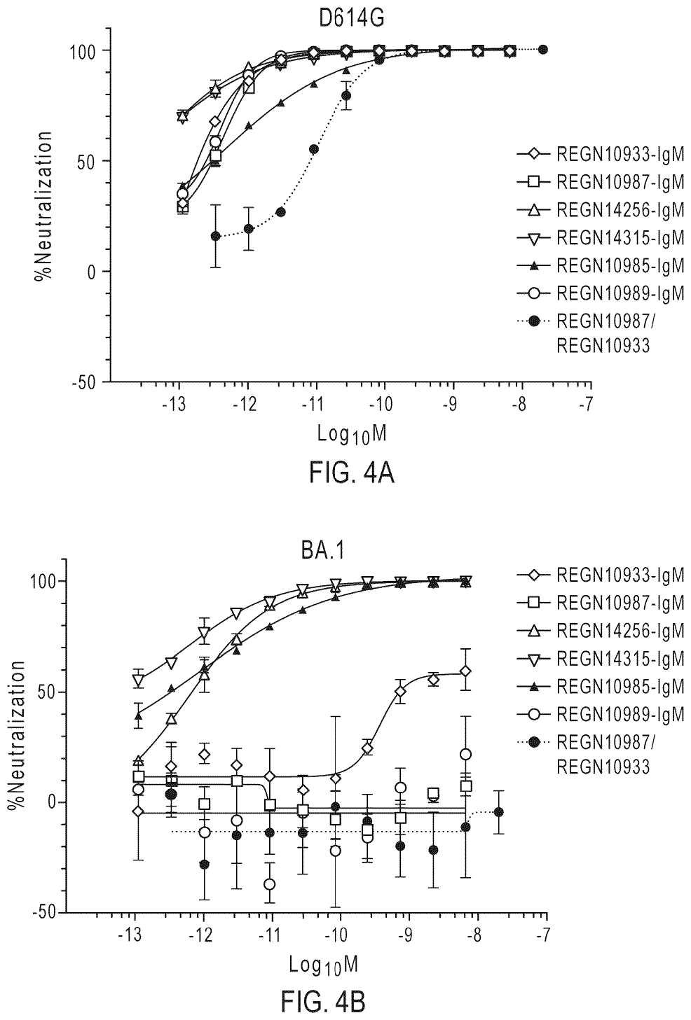

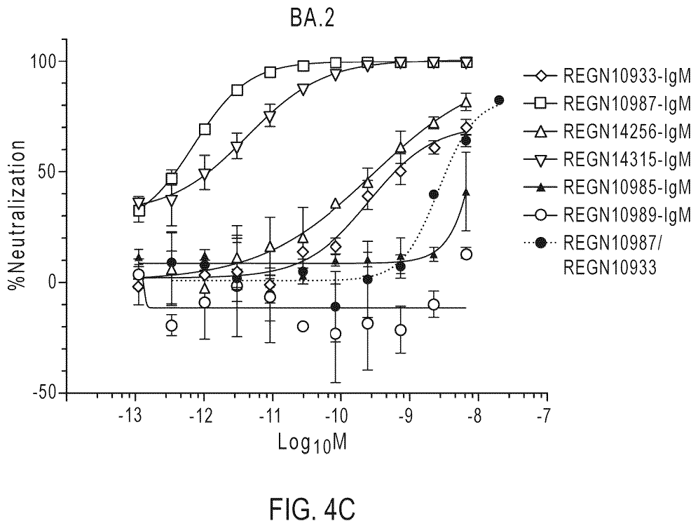

All anti-SARS-CoV2 IgM molecules had neutralization activity against the pseudovirus D614G variant, albeit with different potencies (FIG. 4A and Table 7). Nevertheless, all IgM molecules were associated with higher pseudovirus neutralization potencies than REGEN-COV.

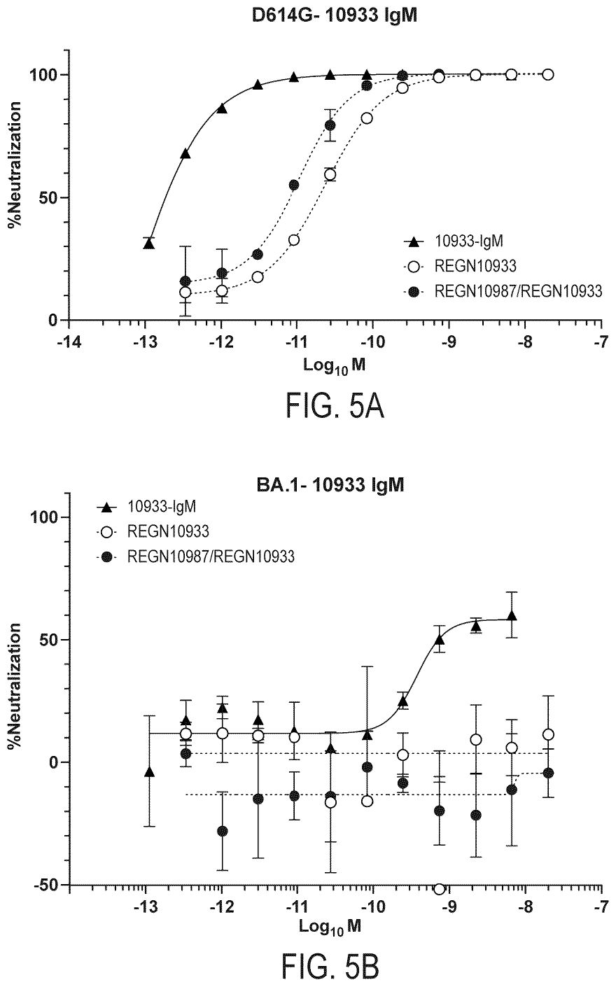

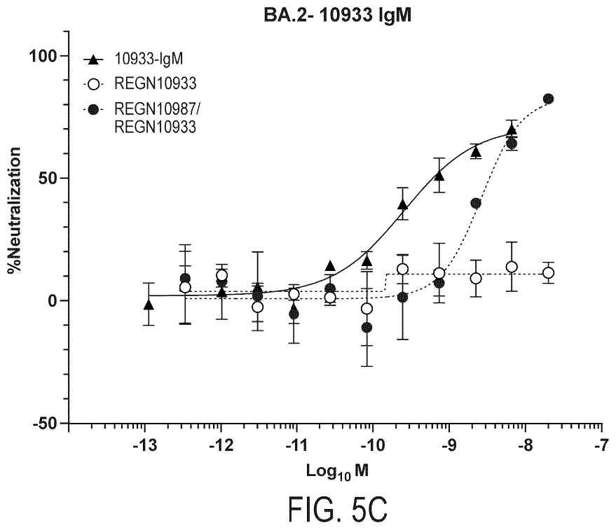

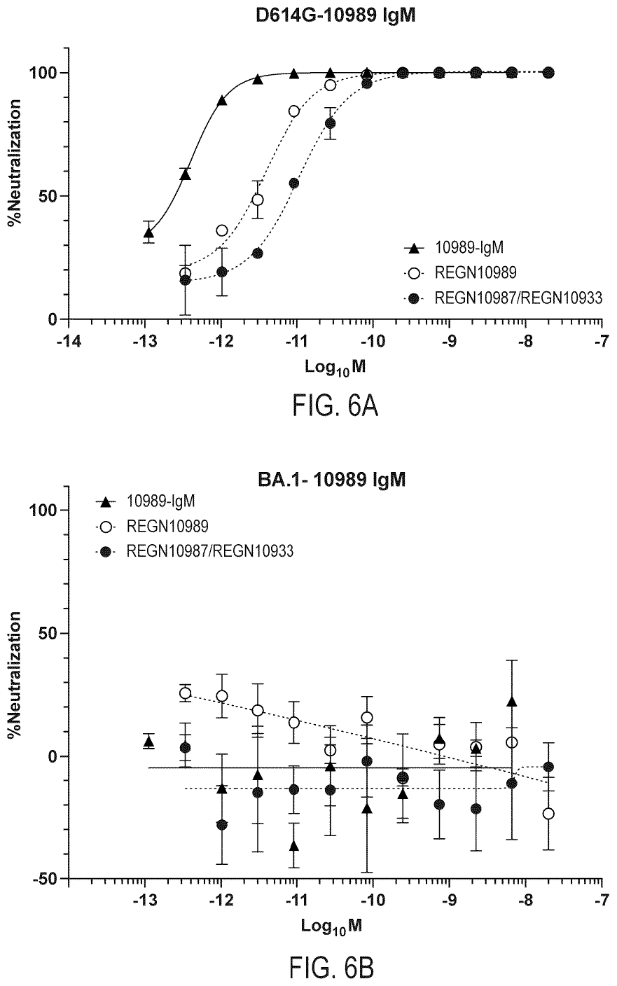

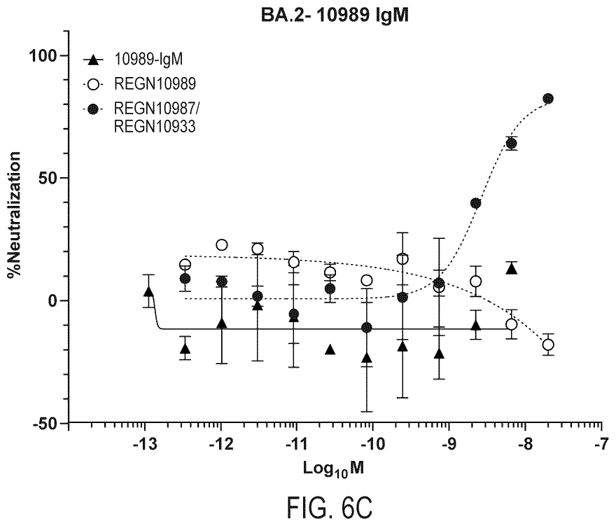

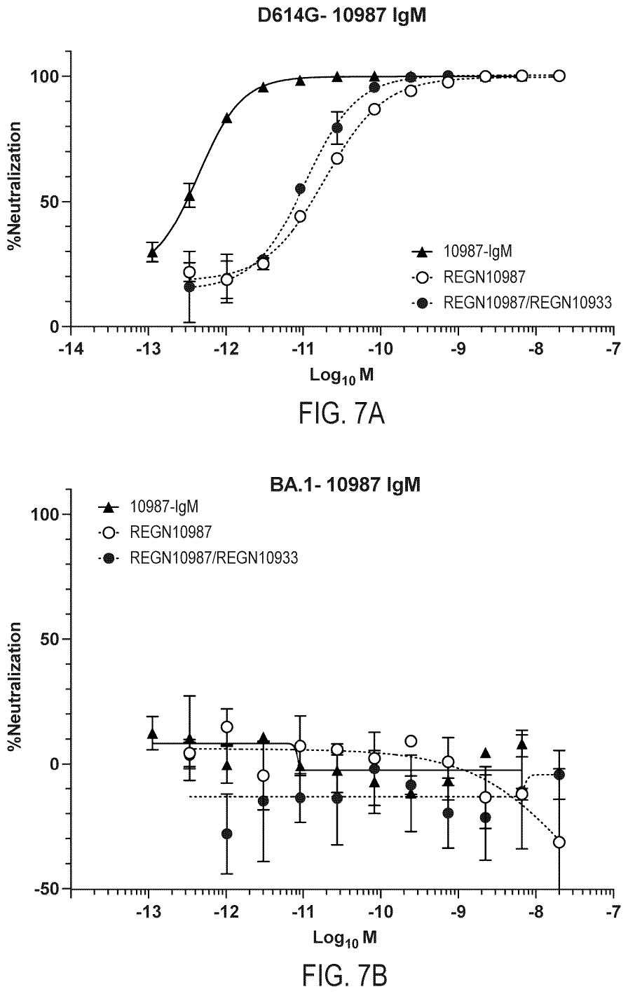

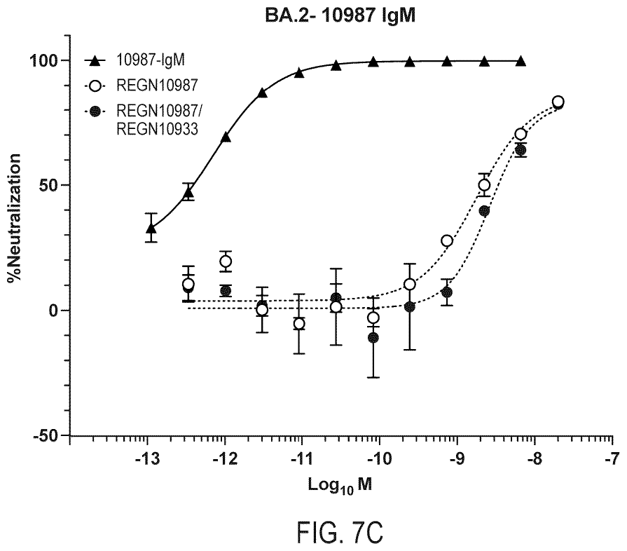

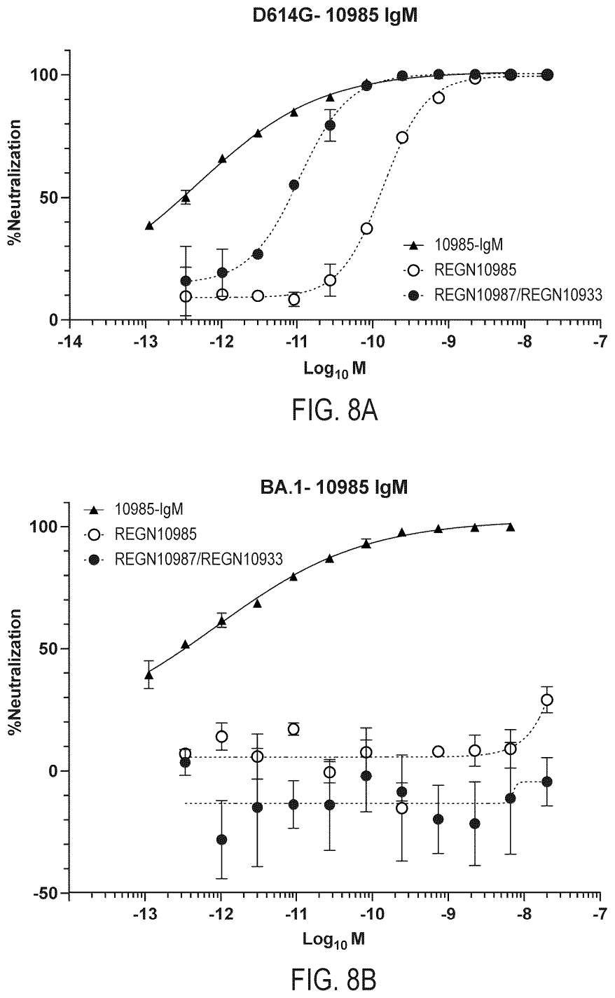

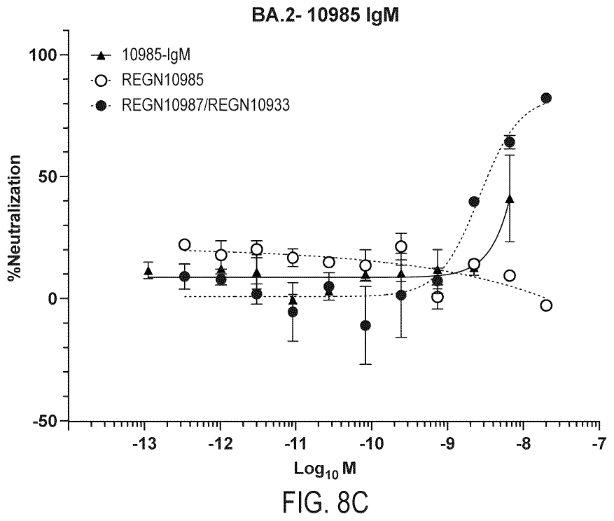

REGEN-COV lost neutralization activity completely against BA. 1 and only retained weak neutralization against BA.2 (FIGS. 4B and 4C, and Table 7). However, the neutralization activities of individual anti-SARS-CoV-2 IgM against BA. 1 and BA.2 variants are varied. For instance, REGN10933 based IgM demonstrated enhanced neutralization over parental IgG and REGEN-COV in D614G, BA. 1, and BA.2 variants. However, 10933 IgM failed to restore the full potency (defined as IC50 against D614G by REGEN-COV) against BA.1 and BA.2 (FIGS. 5A, 5B, and 5C). 10989-IgM demonstrated enhanced neutralization over parental IgG and REGEN-COV in D614G. It lost activity completely against BA. 1 and BA.2 (FIGS. 6A, 6B, and 6C). 10987-IgM had better neutralization than parental IgG and REGEN-COV against D614G and BA.2 variants. However, there was no activity against BA. 1 (FIGS. 7A, 7B, and 7C). 10985-IgM displayed elevated neutralization over parental IgG and REGEN-COV in D614G and BA. 1 variants, but the neutralization activity was completely vanished against BA.2 (FIGS. 8A, 8B, and 8C).

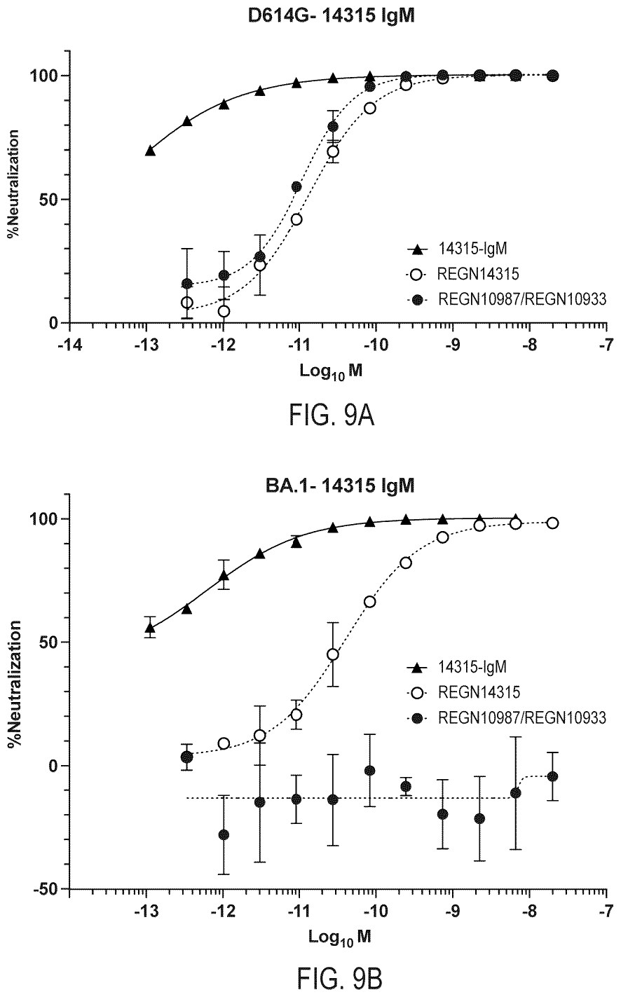

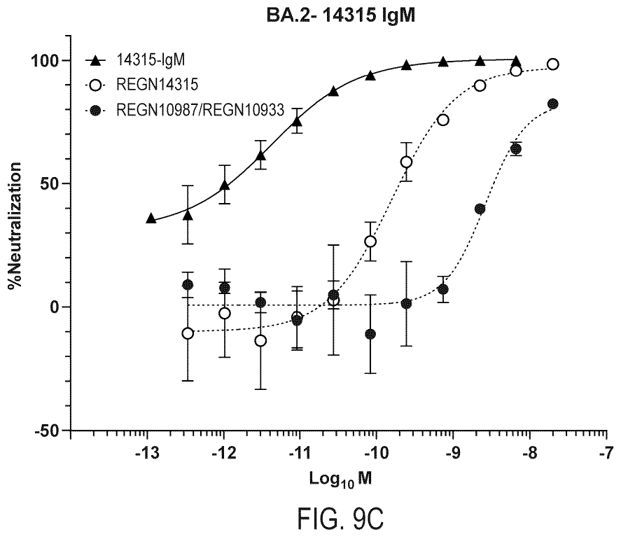

14315-IgM was the only RBD-based IgM that was associated with a high neutralization potency against both variants with IC50s better than the full potency (against D614G) of REGEN-COV (FIGS. 9A, 9B and 9C, and Table 7).

TABLE 7
Summary of Neutralization Activity Against SARS-CoV-2 Pseudovirus
D614G, BA.1 and BA.2 Among Six RBD-Binding IgMs

| | | | | | | Fold Change Over | | |
|---|---|---|---|---|---|---|---|---|
| | | | | | | REGN10933/10987 | | |
| | | MW | IC50 (M) | | | Against D614G | | |
| Format | REGN/AF ID | (kDa) | D614G | BA.1 | BA.2 | D614G | BA.1 | BA.2 |
| IgM | 10933-IgM | 870 | 7.47E−14 | 3.73E−10 | 2.57E−10 | 0.01 | 34.94 | 24.06 |
| | 10985-IgM | 870 | 4.77E−13 | 8.55E−13 | NC | 0.04 | 0.08 | NC |
| | 10987-IgM | 870 | 4.40E−13 | NC | 7.08E−13 | 0.04 | NC | 0.07 |
| | 10989-IgM | 870 | 4.00E−13 | NC | NC | 0.04 | NC | NC |
| | 14256-IgM | 870 | 4.62E−14 | 6.26E−13 | 2.87E−10 | 0.004 | 0.06 | 26.94 |
| | 14315-IgM | 870 | 5.29E−14 | 6.27E−13 | 4.22E−12 | 0.005 | 0.06 | 0.40 |
| IgG | 10933 + | 150 | 1.07E−11 | NC | 2.65E−09 | 1.00 | 669.89 | 248.59 |
| control | 10987 | | | | | | | |
| | 10933 | 150 | 2.37E−11 | NC | NC | 2.23 | NC | NC |
| | 10985 | 150 | 1.37E−10 | NC | NC | 12.87 | NC | NC |
| | 10987 | 150 | 1.91E−11 | NC | 1.77E−09 | 1.79 | NC | 166.23 |
| | 10989 | 150 | 3.84E−12 | NC | NC | 0.36 | NC | NC |
| | 14256 | 150 | 2.45E−11 | NC | NC | 2.30 | NC | NC |
| | 14315 | 150 | 1.33E−11 | 4.04E−11 | 1.60E−10 | 1.24 | 3.79 | 14.98 |

NC: Not Calculated

### 8.4. Example 3: Neutralization Activity of the non-RB-targeted Anti-SARS-CoV-2 IgM

One of the seven anti-SARS-CoV-2 IgMs generated in Example 1, 14287-IgM was derived from a parental antibody that binds to a non-RBD epitope on the spike protein. It was produced and tested in virus neutralization assays as described in Section 8.1.1. and Section 8.1.2., respectively. For the pseudovirus based neutralization assays, alongside with REGEN-COV, REGN14287 was included as the parental IgG control with the same Fab moiety.

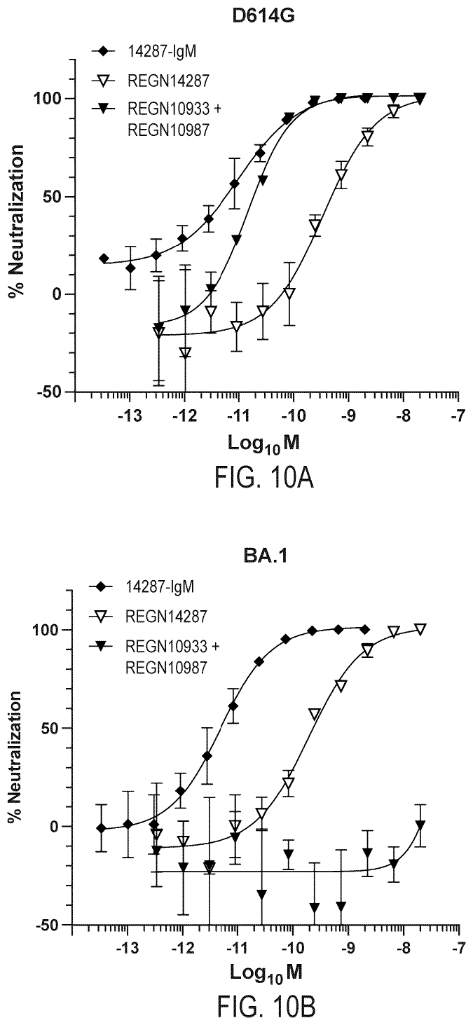

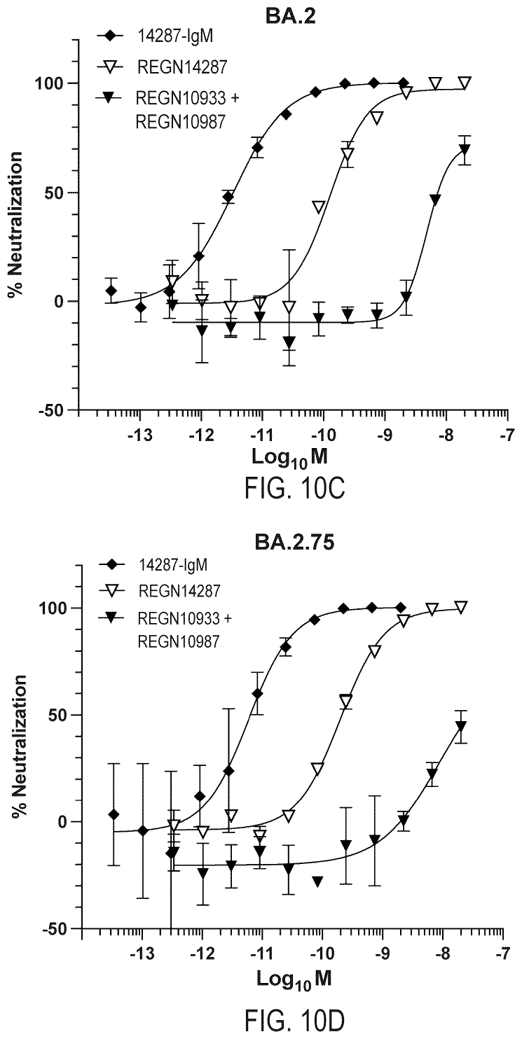

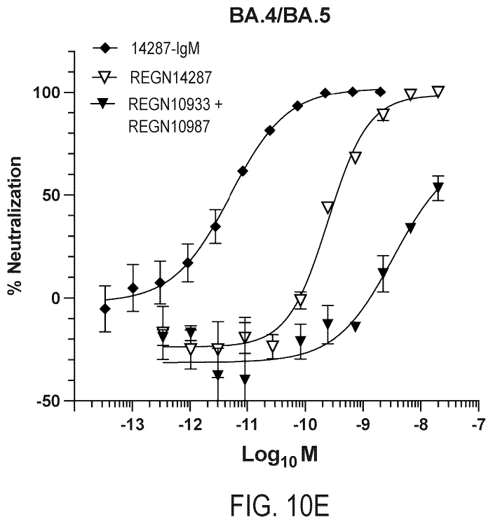

14287-IgM demonstrated desired broad and potent neutralization activity across D614G and Omicron BA.1, BA.2, BA.2.75, and BA.4/BA.5 with IC50 values between 3.2-9.6 E⁻¹² M, 2-5-fold more potent than REGEN-COV against D614G. It also displays 10-fold potency enhancement over parental REGN14287 IgG (FIG. 10A-10E, and Table 8).

TABLE 8
Summary of Neutralization Activity Against SARS-CoV-2 Pseudovirus D614G,
BA.1, BA.2, BA2.75 and BA.4/BA.5 for Non-RBD-Binding 14287-IgM

| | | | | | | | | Fold Change Over | | | | |
|---|---|---|---|---|---|---|---|---|---|---|---|---|
| | | | | | | | | REGN10933/10987 | | | | |
| | | | IC50 (M) | | | | | Against D614G | | | | |
| | REGN/ | MW | | | | | BA.4/ | | | | | BA.4/ |
| Format | AF ID | (kDa) | D614G | BA.1 | BA.2 | BA.2.75 | BA.5 | D614G | BA.1 | BA.2 | BA.2.75 | BA.5 |
| IgM | 14287-IgM | 870 | 9.59E−12 | 4.84E−12 | 3.22E−12 | 5.87E−12 | 5.04E−12 | 0.65 | 0.33 | 0.22 | 0.40 | 0.34 |
| IgG | 10933 + | 150 | 1.47E−11 | NC | 4.77E−09 | NC | 3.37E−09 | 1.00 | NC | 325.51 | NC | 229.67 |
| control | 10987 | | | | | | | | | | | |
| | 14287 | 150 | *9.06E−11 | *4.81E−11 | *2.84E−11 | *5.78E−11 | *5.00E−11 | 7.02 | 3.28 | 1.94 | 3.94 | 3.41 |

NC: Not Calculated;
*Geometric mean of two or three repeated experiments

### 8.5. Example 4: Neutralization Activity of 14315 and 14287-IgMs Against Currently Circulating Omicron Variant BQ.1

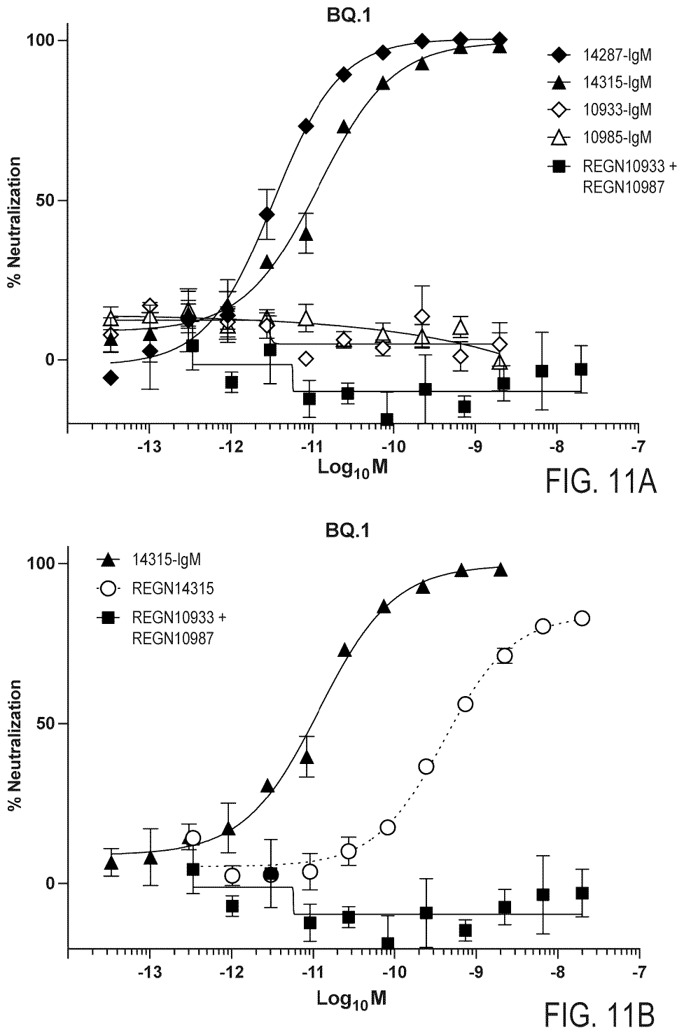

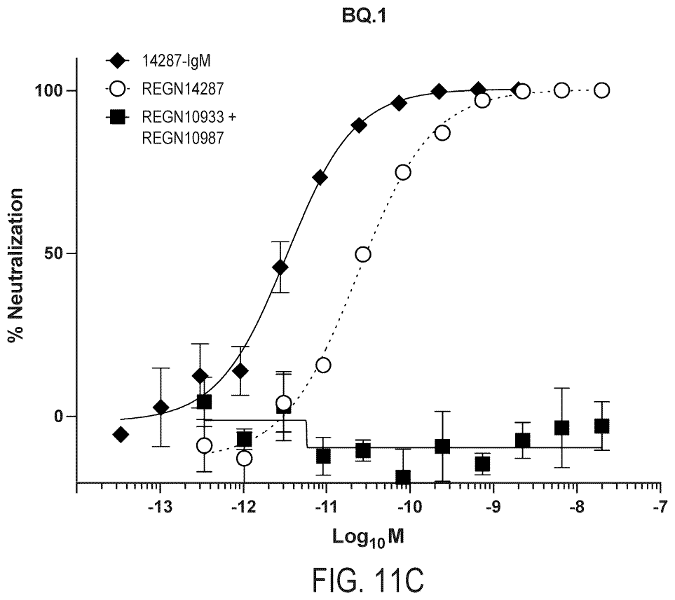

To examine whether the lead IgMs with the desired breadth and potency could neutralize a currently circulating variant, the neutralization assay described in Section 8.1.2 was used to test the efficacy of 14315 and 14287-IgMs with Omicron BQ. 1 variant. In addition, 10933-IgM, 10985-IgM, 14287-IgG (REGN14287), 14315-IgG (REGN14315), and REGEN-COV were included as controls. Two broad IgM neutralizers, 14315-IgM and 14287-IgM, showed potent inhibition against pseudovirus Omicron BQ. 1 variant, with IC50 of 1.2 E⁻¹¹ and 3.3 E⁻¹² M, respectively, but 10933-IgM or 10985-IgM did not. Although the parental REGN14315 and REGN14287 IgGs neutralized BQ. 1 variant with potency values of 4.0 E⁻¹⁰ M and 2.4 E⁻¹¹ M, respectively, they were 34- and 2-fold weaker than that of REGEN-COV against D614G (FIGS. 11B, and 11C). The corresponding 14315-IgM boosted the activity to the full potency of REGEN-COV and 14287-IgM displayed 3 to 4-fold higher activity than REGEN-COV (FIGS. 11A, 11B, and 11C). Additionally, both IgMs were 3 to 12-fold more potent than corresponding parental IgGs against BQ. 1 (FIG. 11B, 11C and Table 9).

These findings indicate that the combination of multivalency and targeted epitopes on SARS-CoV2 spike protein in the format of IgM plays a critical role in achieving higher potency and broader protection than their parental IgGs with the corresponding Fab moiety. Increase in valency alone for IgGs that have weakened or lost ability to neutralize does not guarantee an activity enhancement against a specific variant. Even if an increase in activity is observed, it may not be fully restored to desired potency (e.g., IC50 of REGEN-COV against D614G).

TABLE 9
Summary of Neutralization Activity Against BQ.1 Variant by Two IgMs

| | | | | | Fold Change Over | |
|---|---|---|---|---|---|---|
| | | | | | REGN10933/10987 | |
| | REGN/ | MW | IC50 (M) | | Against D614G | |
| Format | AF ID | (kDa) | D614G | BQ.1 | D614G | BQ.1 |
| IgM | 14287-IgM | 870 | 1.01E-11 | 3.30E-12 | 0.84 | 0.28 |
| | 14315-IgM | 870 | 1.30E-12 | 1.19E-11 | 0.11 | 0.99 |
| IgG | 14287 | 150 | 7.04E-11 | 2.38E-11 | 5.89 | 1.99 |
| control | 14315 | 150 | 7.90E-12 | 4.01E-10 | 0.66 | 33.53 |
| | 10933 + | 150 | 1.20E-11 | NC | 1.00 | NC |
| | 10987 | | | | | |

NC: Not Calculated

### 8.6. Example 5: SARS-CoV-2 Virus Neutralization Activity of 14287-IgMs Comprising IgG Fc-linked J Chains

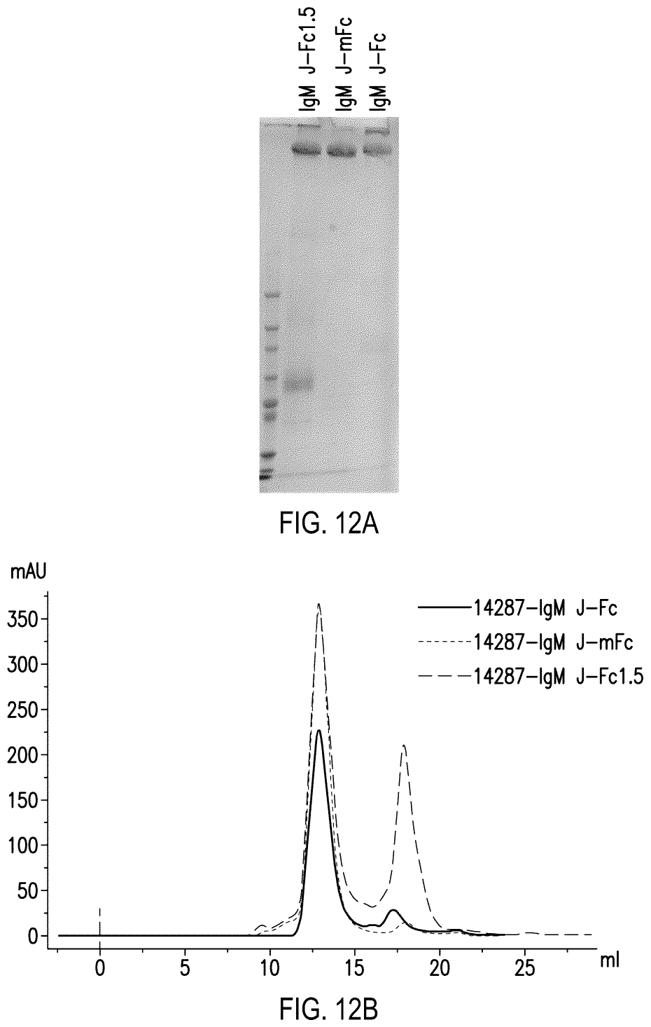

Using 14287-IgM as a starting point, three anti-SARS-CoV-2 IgMs comprising IgG Fc-linked J chains were generated and purified as described in Section 8.1.1. The construct 14287-IgM J-Fc comprised a J chain operably linked to a dimerizing IgG Fc domain; the construct 14287-IgM J-Fc1.5 comprised a J chain operably linked to an Fc1.5 domain; and the construct 14287-IgM J-mFc comprised a J chain operably linked to a non-dimerizing Fc domain. Purified constructs were able to bind to Protein A (FIG. 12A). The SEC purification profiles of all three constructs displayed discrete main peaks (FIG. 12B).

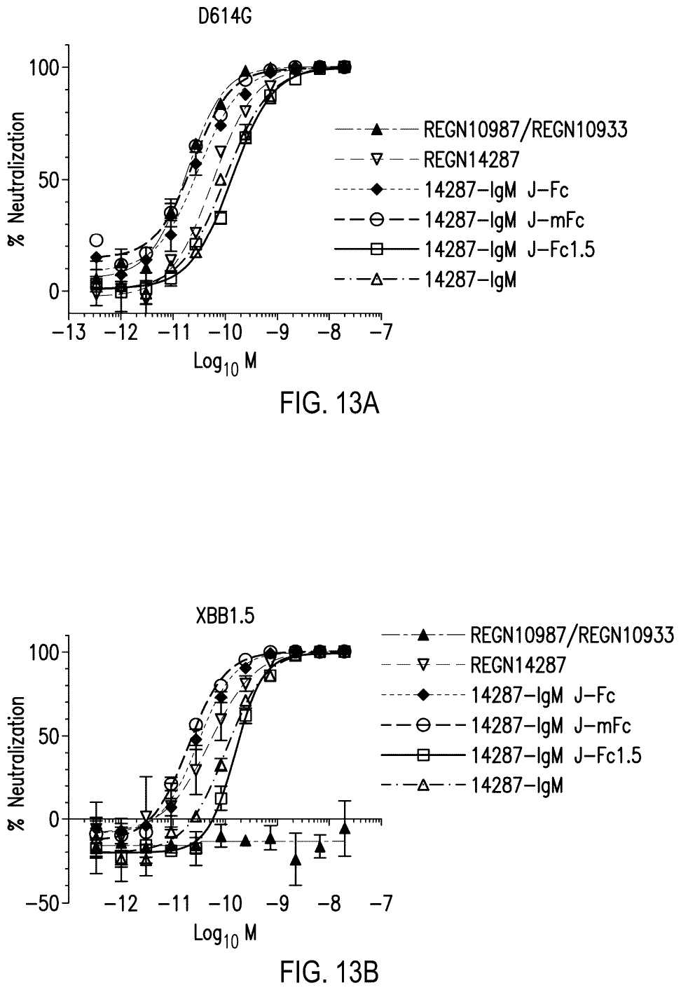

The IgM constructs comprising IgG Fc-linked J chains, 14287-IgM J-Fc, 14287-IgM J-mFc, and 14287-IgM J-Fc1.5, were evaluated in virus neutralization assays as described Section 8.1.2. REGN14287-IgG, 14287-IgM, and REGEN-COV (REGN10987/REGN10933) were also included for comparison. Results showed that all three IgM constructs comprising IgG Fc linked J chains displayed varying degrees of potency against the SARS-CoV-2 pseudovirus (FIG. 13A and Table 10) and the XBB1.5 variant (FIG. 13B and Table 10).

TABLE 10
Summary of Neutralization Activity Against XBB.1.5 Variant
by IgM Constructs Comprising IgG Fc Linked J Chains

| | | IC50 (M) | |
|---|---|---|---|
| Format | REGN/AF ID | D614G | XBB1.5 |
| IgM | 14287-IgM | 9.99E−11 | 1.01E−10 |
| | 14287-IgM J-Fc | 3.06E−11 | 3.02E−11 |
| | 14287-IgM J-mFc | 2.41E−11 | 1.98E−11 |
| | 14287-IgM J-Fc1.5 | 1.35E−10 | 1.56E−10 |
| IgG | 14287-IgG | 5.67E−11 | 5.14E−11 |
| | 10933 + 10987 (REGEN-COV) | 1.88E−11 | NC |

NC: Not Calculated

### 9. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

What is claimed is:

1. A multivalent anti-spike protein binding molecule comprising at least 5 anti-spike protein antigen-binding domains (ABDs) operably linked by one or more multimerization moieties.

2. The multivalent anti-spike protein binding molecule of claim 1, which comprises at least 10 anti-spike protein ABDs.

3. The multivalent anti-spike protein binding molecule of claim 1 or claim 2, which is decavalent or dodecavalent.

4. The multivalent anti-spike protein binding molecule of any one of claims 1 to 3, wherein the antigen-binding domains (ABDs) are human or humanized.

5. The multivalent anti-spike protein binding molecule of any one of claims 1 to 4, wherein the antigen-binding domains (ABDs) are Fabs.

6. The multivalent anti-spike protein binding molecule of any one of claims 1 to 5, wherein one or more (or all) ABDs are neutralizing.

7. The multivalent anti-spike protein binding molecule of any one of claims 1 to 6, which is monospecific.

8. The multivalent anti-spike protein binding molecule of any one of claims 1 to 7, in which the antigen-binding domains (ABDs) are all the same.

9. The multivalent anti-spike protein binding molecule of any one of claims 1 to 6, which is multispecific.

10. The multivalent anti-spike protein binding molecule of claim 9, which is bispecific.

11. The multivalent anti-spike protein binding molecule of claim 9 or claim 10, which comprises two types of ABDs.

12. The multivalent anti-spike protein binding molecule of any one of claims 1 to 11, wherein the one or more multimerization moieties comprise an Fc domain.

13. The multivalent anti-spike protein binding molecule of claim 11, wherein both types of ABDs bind to spike protein.

14. The multivalent anti-spike protein binding molecule of claim 11, wherein one type of ABD binds to spike protein and the other type of ABD binds to a different target.

15. The multivalent anti-spike protein binding molecule of claim 12, wherein the Fc domain is an IgM Fc domain.

16. The multivalent anti-spike protein binding molecule of claim 15, wherein the Fc domain comprises a Cμ3 domain and a Cμ4 domain.

17. The multivalent anti-spike protein binding molecule of claim 15 or claim 16, wherein the Fc domain comprises a Cμ2 domain.

18. The multivalent anti-spike protein binding molecule of any one of claims 15 to 17, which is a pentamer.

19. The multivalent anti-spike protein binding molecule of claim 18, which is a pentamer of five dimers, each dimer comprising two polypeptides, each polypeptide comprising an anti-spike protein ABD and an IgM Fc domain.

20. The multivalent anti-spike protein binding molecule of claim 18 or claim 19, which is a homopentamer.

21. The multivalent anti-spike protein binding molecule of any one of claims 18 to 20, in which a portion or all Cμ3 and/or Cμ4 domains are disulfide linked.

22. The multivalent anti-spike protein binding molecule of any one of claims 18 to 21, which comprises a J chain.

23. The multivalent anti-spike protein binding molecule of claim 22, wherein the J chain is operably linked to an IgG Fc domain.

24. The multivalent anti-spike protein binding molecule of claim 23, wherein the IgG Fc domain and the J chain are connected via a linker.

25. The multivalent anti-spike protein binding molecule of claim 23 or 24, wherein the IgG Fc domain is a non-dimerizing Fc domain.

26. The multivalent anti-spike protein binding molecule of claim 23 or 24, wherein the IgG Fc domain is an Fc 1.5 domain.

27. The multivalent anti-spike protein binding molecule of any one of claims 15 to 17, which is a hexamer.

28. The multivalent anti-spike protein binding molecule of claim 27, which is a hexamer of six dimers, each dimer comprising two polypeptides, each polypeptide comprising an anti-spike protein ABD and an IgM Fc domain.

29. The multivalent anti-spike protein binding molecule of claim 27 or claim 28, which is a homohexamer.

30. The multivalent anti-spike protein binding molecule of any one of claims 27 to 29, in which a portion or all Cμ3 and/or Cμ4 domains are disulfide linked.

31. The multivalent anti-spike protein binding molecule of any one of claims 27 to 30, which lacks a J chain.

32. The multivalent anti-spike protein binding molecule of any one of claims 1 to 31, wherein one or more (or all) ABDs comprise the CDR-H1, CDR-H2, CDR-H3, CDR-L1, CDR-L2, and CDR-L3 sequences of an antibody set forth in Table 4.

33. The multivalent anti-spike protein binding molecule of any one of claims 1 to 31, wherein one or more (or all) ABDs comprise VH and VL sequences of an antibody set forth in Table 4.

34. The multivalent anti-spike protein binding molecule of any one of claims 1 to 31, wherein one or more (or all) ABDs comprise:
(a) a VH comprising CDR-H1, CDR-H2, and CDR-H3 having the amino acid sequences of SEQ ID NOs: 579, 580, and 581, respectively; and
(b) a VL comprising CDR-L1, CDR-L2, and CDR-L3 having the amino acid sequences of SEQ ID NOs: 398, 372, and 583, respectively.

35. A nucleic acid or plurality of nucleic acids encoding the multivalent anti-spike protein binding molecule of any one of claims 1 to 34.

36. A host cell engineered to express the multivalent anti-spike protein binding molecule of any one of claims 1 to 34 or the nucleic acid(s) of claim 35.

37. A method of producing the multivalent anti-spike protein binding molecule of any one of claims 1 to 34, comprising culturing the host cell of claim 36 and recovering the multivalent anti-spike protein binding molecule expressed thereby.

38. A pharmaceutical composition comprising the multivalent anti-spike protein binding molecule of any one of claims 1 to 34 and an excipient.

39. A method of treating a coronavirus disease, comprising administering to a subject in need thereof the multivalent anti-spike protein binding molecule of any one of claims 1 to 34 or the pharmaceutical composition of claim 38.

40. A method of inhibiting an interaction between a RBD of a coronavirus and cellular ACE2, comprising administering to a subject in need thereof the multivalent anti-spike protein binding molecule of any one of claims 1 to 34 or the pharmaceutical composition of claim 38.

41. A method of neutralizing a coronavirus spike protein in vivo, comprising administering to a subject in need thereof the multivalent anti-spike protein binding molecule of any one of claims 1 to 34 or the pharmaceutical composition of claim 38.

42. A method of inhibiting protease-mediated cleavage (e.g., TMPRSS2-mediated cleavage) of a coronavirus spike protein in vivo, comprising administering to a subject in need thereof the multivalent anti-spike protein binding molecule of any one of claims 1 to 34 or the pharmaceutical composition of claim 38.

43. A method of inhibiting viral entry of a coronavirus into a host cell in a subject, comprising administering to a subject in need thereof the multivalent anti-spike protein binding molecule of any one of claims 1 to 34 or the pharmaceutical composition of claim 38.

44. A method of inhibiting reproduction of a coronavirus spike protein in a host cell in a subject, comprising administering to a subject in need thereof the multivalent anti-spike protein binding molecule of any one of claims 1 to 34 or the pharmaceutical composition of claim 38.

45. A method reducing the severity of coronavirus infection, comprising administering to a subject in need thereof the multivalent anti-spike protein binding molecule of any one of claims 1 to 34 or the pharmaceutical composition of claim 38.

46. A method of reducing the viral load of a coronavirus, comprising administering to a subject in need thereof the multivalent anti-spike protein binding molecule of any one of claims 1 to 34 or the pharmaceutical composition of claim 38.

47. A method of preventing disease progression in a subject with a coronavirus infection, comprising administering to a subject in need thereof the multivalent anti-spike protein binding molecule of any one of claims 1 to 34 or the pharmaceutical composition of claim 38.

48. A method of reducing the duration of a coronavirus infection, comprising administering to a subject in need thereof the multivalent anti-spike protein binding molecule of any one of claims 1 to 34 or the pharmaceutical composition of claim 38.

49. A method of reducing the risk of severe disease or death in a subject with a coronavirus infection, comprising administering to a subject in need thereof the multivalent anti-spike protein binding molecule of any one of claims 1 to 34 or the pharmaceutical composition of claim 38.

50. The method of any one of claims 39 to 49, wherein the coronavirus is SARS-CoV-2.


SEQUENCE LISTING (752 sequences)

SEQ ID NO: 1 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 2 - protein, 137 aa, synthetic construct
QEDERIVLVDNKCKCARITSRIIRSSEDPNEDIVERNIRIIVPLNNRENIADPTSPLRTRFVYHLSDLCKKCDPTEVELDNQIVTATQSNICDEDSATETCYTYDRNKCYTAVVPLVYGGETKMVETALTPDACYPD

SEQ ID NO: 3 - protein, 453 aa, Homo sapiens [sequence omitted]

SEQ ID NO: 4 - protein, 101 aa, Homo sapiens
DVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVD

SEQ ID NO: 5 - protein, 124 aa, Homo sapiens
DVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 6 - protein, 207 aa, Homo sapiens
VFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVD

SEQ ID NO: 7 - protein, 230 aa, Homo sapiens
VFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 8 - protein, 317 aa, Homo sapiens
SVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVD

SEQ ID NO: 9 - protein, 340 aa, Homo sapiens
SVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 10 - protein, 120 aa, synthetic construct
QVQLVESGGGLVKPGGSLRLSCAASGFTFSDYYMSWIRQAPGKGLEWVSYITYSGSTIYYADSVKGRFTISRDNAKSSLYLQMNSLRAEDTAVYYCARDRGTTMVPFDYWGQGTLVTVSS

SEQ ID NO: 11 - protein, 8 aa, synthetic construct
GFTFSDYY

SEQ ID NO: 12 - protein, 8 aa, synthetic construct
ITYSGSTI

SEQ ID NO: 13 - protein, 13 aa, synthetic construct
ARDRGTTMVPFDY

SEQ ID NO: 14 - protein, 107 aa, synthetic construct
DIQMTQSPSSLSASVGDRVTITCQASQDITNYLNWYQQKPGKAPKLLIYAASNLETGVPSRFSGSGSGTDFTFTISGLQPEDIATYYCQQYDNLPLTFGGGTKVEIK

SEQ ID NO: 15 - protein, 6 aa, synthetic construct
QDITNY

SEQ ID NO: 16 - protein, 9 aa, synthetic construct
QQYDNLPLT

SEQ ID NO: 17 - protein, 450 aa, synthetic construct
QVQLVESGGGLVKPGGSLRLSCAASGFTFSDYYMSWIRQAPGKGLEWVSYITYSGSTIYYADSVKGRFTISRDNAKSSLYLQMNSLRAEDTAVYYCARDRGTTMVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 214 aa, synthetic construct
DIQMTQSPSSLSASVGDRVTITCQASQDITNYLNWYQQKPGKAPKLLIYAASNLETGVPSRFSGSGSGTDFTFTISGLQPEDIATYYCQQYDNLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 360 bp, synthetic construct
  source 1..360: mol_type = other DNA
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattacttatagtggtagtaccatatactacgcagactctgtgaagggccgattcaccatctccagggacaacgccaagagctcactgtatctgcaaatgaacagcctgagagccgaggacacggccgtgtattactgtgcgagagatcgcggtacaactatggtcccctttgactactggggccagggaaccctggtcaccgtctcctca

SEQ ID NO: 20 - DNA, 24 bp, synthetic construct
  source 1..24: mol_type = other DNA
ggattcaccttcagtgactactac

SEQ ID NO: 21 - DNA, 24 bp, synthetic construct
  source 1..24: mol_type = other DNA
attacttatagtggtagtaccata

SEQ ID NO: 22 - DNA, 39 bp, synthetic construct
  source 1..39: mol_type = other DNA
gcgagagatcgcggtacaactatggtcccctttgactac

SEQ ID NO: 23 - DNA, 321 bp, synthetic construct
  source 1..321: mol_type = other DNA
gacatccagatgacccagtctccatcctccctgtctgcatctgtaggagacagagtcaccatcacttgccaggcgagtcaggacattaccaactatttaaattggtatcagcagaaaccagggaaagcccctaagctcctgatctacgctgcatccaatttggaaacaggggtcccatcaaggttcagtggaagtggatctgggacagattttactttcaccatcagcggcctgcagcctgaagatattgcaacatattactgtcaacagtatgataatctccctctcactttcggcggagggaccaaggtggagatcaaa

SEQ ID NO: 24 - DNA, 18 bp, synthetic construct
  source 1..18: mol_type = other DNA
caggacattaccaactat

SEQ ID NO: 25 - DNA, 27 bp, synthetic construct
  source 1..27: mol_type = other DNA
caacagtatgataatctccctctcact

SEQ ID NO: 26 - DNA, 1353 bp, synthetic construct
  source 1..1353: mol_type = other DNA
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattacttatagtggtagtaccatatactacgcagactctgtgaagggccgattcaccatctccagggacaacgccaagagctcactgtatctgcaaatgaacagcctgagagccgaggacacggccgtgtattactgtgcgagagatcgcggtacaactatggtcccctttgactactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 27 - DNA, 645 bp, synthetic construct
  source 1..645: mol_type = other DNA
gacatccagatgacccagtctccatcctccctgtctgcatctgtaggagacagagtcaccatcacttgccaggcgagtcaggacattaccaactatttaaattggtatcagcagaaaccagggaaagcccctaagctcctgatctacgctgcatccaatttggaaacaggggtcccatcaaggttcagtggaagtggatctgggacagattttactttcaccatcagcggcctgcagcctgaagatattgcaacatattactgtcaacagtatgataatctccctctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 28 - protein, 120 aa, synthetic construct
EVQLVESGGGLVKPGGSLRLSCAASGITFSNAWMSWVRQAPGKGLEWVGRIKSKTDGGTTDYAAPVKGRFTISRDDSKNTLYLQMNSLKTEDTAVYYCTTARWDWYFDLWGRGTLVTVSS

SEQ ID NO: 29 - protein, 8 aa, synthetic construct
GITFSNAW

SEQ ID NO: 30 - protein, 10 aa, synthetic construct
IKSKTDGGTT

SEQ ID NO: 31 - protein, 11 aa, synthetic construct
TTARWDWYFDL

SEQ ID NO: 32 - protein, 107 aa, synthetic construct
DIQMTQSPSSLSASVGDRVTITCQASQDIWNYINWYQQKPGKAPKLLIYDASNLKTGVPSRFSGSGSGTDFTFTISSLQPEDIATYYCQQHDDLPPTFGQGTKVEIK

SEQ ID NO: 33 - protein, 6 aa, synthetic construct
QDIWNY

SEQ ID NO: 34 - protein, 9 aa, synthetic construct
QQHDDLPPT

SEQ ID NO: 35 - protein, 450 aa, synthetic construct
EVQLVESGGGLVKPGGSLRLSCAASGITFSNAWMSWVRQAPGKGLEWVGRIKSKTDGGTTDYAAPVKGRFTISRDDSKNTLYLQMNSLKTEDTAVYYCTTARWDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 214 aa, synthetic construct
DIQMTQSPSSLSASVGDRVTITCQASQDIWNYINWYQQKPGKAPKLLIYDASNLKTGVPSRFSGSGSGTDFTFTISSLQPEDIATYYCQQHDDLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - DNA, 360 bp, synthetic construct
  source 1..360: mol_type = other DNA
gaggtgcagctggtggagtctgggggaggcttggtaaagcctggggggtcccttagactctcctgtgcagcctctggaatcactttcagtaacgcctggatgagttgggtccgccaggctccagggaaggggctggagtgggttggccgtattaaaagcaaaactgatggtgggacaacagactacgccgcacccgtgaaaggcagattcaccatctcaagagatgattcaaaaaacacgctgtatctacaaatgaacagcctgaaaaccgaggacacagccgtgtattactgtaccacagcgaggtgggactggtacttcgatctctggggccgtggcaccctggtcactgtctcctca

SEQ ID NO: 38 - DNA, 24 bp, synthetic construct
  source 1..24: mol_type = other DNA
ggaatcactttcagtaacgcctgg

SEQ ID NO: 39 - DNA, 30 bp, synthetic construct
  source 1..30: mol_type = other DNA
attaaaagcaaaactgatggtgggacaaca

SEQ ID NO: 40 - DNA, 33 bp, synthetic construct
  source 1..33: mol_type = other DNA
accacagcgaggtgggactggtacttcgatctc

SEQ ID NO: 41 - DNA, 321 bp, synthetic construct
  source 1..321: mol_type = other DNA
gacatccagatgacccagtctccatcctccctgtctgcatctgtaggagacagagtcaccatcacttgccaggcgagtcaggacatttggaattatataaattggtatcagcagaaaccagggaaggcccctaagctcctgatctacgatgcatccaatttgaaaacaggggtcccatcaaggttcagtggaagtggatctgggacagattttactttcaccatcagcagcctgcagcctgaagatattgcaacatattactgtcaacagcatgatgatctccctccgaccttcggccaagggaccaaggtggaaatcaaa

SEQ ID NO: 42 - DNA, 18 bp, synthetic construct
  source 1..18: mol_type = other DNA
caggacatttggaattat

SEQ ID NO: 43 - DNA, 27 bp, synthetic construct
  source 1..27: mol_type = other DNA
caacagcatgatgatctccctccgacc

SEQ ID NO: 44 - DNA, 1353 bp, synthetic construct
  source 1..1353: mol_type = other DNA
gaggtgcagctggtggagtctgggggaggcttggtaaagcctggggggtcccttagactctcctgtgcagcctctggaatcactttcagtaacgcctggatgagttgggtccgccaggctccagggaaggggctggagtgggttggccgtattaaaagcaaaactgatggtgggacaacagactacgccgcacccgtgaaaggcagattcaccatctcaagagatgattcaaaaaacacgctgtatctacaaatgaacagcctgaaaaccgaggacacagccgtgtattactgtaccacagcgaggtgggactggtacttcgatctctggggccgtggcaccctggtcactgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 45 - DNA, 645 bp, synthetic construct
  source 1..645: mol_type = other DNA
gacatccagatgacccagtctccatcctccctgtctgcatctgtaggagacagagtcaccatcacttgccaggcgagtcaggacatttggaattatataaattggtatcagcagaaaccagggaaggcccctaagctcctgatctacgatgcatccaatttgaaaacaggggtcccatcaaggttcagtggaagtggatctgggacagattttactttcaccatcagcagcctgcagcctgaagatattgcaacatattactgtcaacagcatgatgatctccctccgaccttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 46 - protein, 120 aa, synthetic construct
QVQLVESGGGVVQPGRSLRLSCAASGFTFSNYAMYWVRQAPGKGLEWVAVISYDGSNKYYADSVKGRFTISRDNSKNTLYLQMNSLRTEDTAVYYCASGSDYGDYLLVYWGQGTLVTVSS

SEQ ID NO: 47 - protein, 8 aa, synthetic construct
GFTFSNYA

SEQ ID NO: 48 - protein, 8 aa, synthetic construct
ISYDGSNK

SEQ ID NO: 49 - protein, 13 aa, synthetic construct
ASGSDYGDYLLVY

SEQ ID NO: 50 - protein, 110 aa, synthetic construct
QSALTQPASVSGSPGQSITISCTGTSSDVGGYNYVSWYQQHPGKAPKLMIYDVSKRPSGVSNRFSGSKSGNTASLTISGLQSEDEADYYCNSLTSISTWVFGGGTKLTVL

SEQ ID NO: 51 - protein, 9 aa, synthetic construct
SSDVGGYNY

SEQ ID NO: 52 - protein, 10 aa, synthetic construct
NSLTSISTWV

SEQ ID NO: 53 - protein, 450 aa, synthetic construct
QVQLVESGGGVVQPGRSLRLSCAASGFTFSNYAMYWVRQAPGKGLEWVAVISYDGSNKYYADSVKGRFTISRDNSKNTLYLQMNSLRTEDTAVYYCASGSDYGDYLLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 216 aa, synthetic construct
QSALTQPASVSGSPGQSITISCTGTSSDVGGYNYVSWYQQHPGKAPKLMIYDVSKRPSGVSNRFSGSKSGNTASLTISGLQSEDEADYYCNSLTSISTWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 55 - DNA, 360 bp, synthetic construct
  source 1..360: mol_type = other DNA
caggtgcagctggtggagtctgggggaggcgtggtccagcctgggaggtccctgagactctcctgtgcagcctctggattcaccttcagtaactatgctatgtactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagaactgaggacacggctgtgtattactgtgcgagtggctccgactacggtgactacttattggtttactggggccagggaaccctggtcaccgtctcctca

SEQ ID NO: 56 - DNA, 24 bp, synthetic construct
  source 1..24: mol_type = other DNA
ggattcaccttcagtaactatgct

SEQ ID NO: 57 - DNA, 24 bp, synthetic construct
  source 1..24: mol_type = other DNA
atatcatatgatggaagtaataaa

SEQ ID NO: 58 - DNA, 39 bp, synthetic construct
  source 1..39: mol_type = other DNA
gcgagtggctccgactacggtgactacttattggtttac

SEQ ID NO: 59 - DNA, 330 bp, synthetic construct
  source 1..330: mol_type = other DNA
cagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaagcggccctcaggggtttctaatcgcttctctggctccaagtctggcaacacggcctccctgaccatctctgggctccagtctgaggacgaggctgattattactgcaactctttgacaagcatcagcacttgggtgttcggcggagggaccaagctgaccgtccta

SEQ ID NO: 60 - DNA, 27 bp, synthetic construct
  source 1..27: mol_type = other DNA
agcagtgacgttggtggttataactat

SEQ ID NO: 61 - DNA, 30 bp, synthetic construct
  source 1..30: mol_type = other DNA
aactctttgacaagcatcagcacttgggtg

SEQ ID NO: 62 - DNA, 1353 bp, synthetic construct
  source 1..1353: mol_type = other DNA
caggtgcagctggtggagtctgggggaggcgtggtccagcctgggaggtccctgagactctcctgtgcagcctctggattcaccttcagtaactatgctatgtactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagaactgaggacacggctgtgtattactgtgcgagtggctccgactacggtgactacttattggtttactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 63 - DNA, 651 bp, synthetic construct
  source 1..651: mol_type = other DNA
cagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaagcggccctcaggggtttctaatcgcttctctggctccaagtctggcaacacggcctccctgaccatctctgggctccagtctgaggacgaggctgattattactgcaactctttgacaagcatcagcacttgggtgttcggcggagggaccaagctgaccgtcctaggccagcccaaggccgccccctccgtgaccctgttccccccctcctccgaggagctgcaggccaacaaggccaccctggtgtgcctgatctccgacttctaccccggcgccgtgaccgtggcctggaaggccgactcctcccccgtgaaggccggcgtggagaccaccaccccctccaagcagtccaacaacaagtacgccgcctcctcctacctgtccctgacccccgagcagtggaagtcccaccggtcctactcctgccaggtgacccacgagggctccaccgtggagaagaccgtggcccccaccgagtgctcctga

SEQ ID NO: 64 - protein, 123 aa, synthetic construct
QVQLVQSGAEVKKPGASVKVSCKASGYIFTGYYMHWVRQAPGQGLEWMGWINPNSGGANYAQKFQGRVTLTRDTSITTVYMELSRLRFDDTAVYYCARGSRYDWNQNNWFDPWGQGTLVTVSS

SEQ ID NO: 65 - protein, 8 aa, synthetic construct
GYIFTGYY

SEQ ID NO: 66 - protein, 8 aa, synthetic construct
INPNSGGA

SEQ ID NO: 67 - protein, 16 aa, synthetic construct
ARGSRYDWNQNNWFDP

SEQ ID NO: 68 - protein, 110 aa, synthetic construct
QSALTQPASVSGSPGQSITISCTGTSSDVGTYNYVSWYQQHPGKAPKLMIFDVSNRPSGVSDRFSGSKSGNTASLTISGLQAEDEADYYCSSFTTSSTVVFGGGTKLTVL

SEQ ID NO: 69 - protein, 9 aa, synthetic construct
SSDVGTYNY

SEQ ID NO: 70 - protein, 10 aa, synthetic construct
SSFTTSSTVV

SEQ ID NO: 71 - protein, 453 aa, synthetic construct
QVQLVQSGAEVKKPGASVKVSCKASGYIFTGYYMHWVRQAPGQGLEWMGWINPNSGGANYAQKFQGRVTLTRDTSITTVYMELSRLRFDDTAVYYCARGSRYDWNQNNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 216 aa, synthetic construct
QSALTQPASVSGSPGQSITISCTGTSSDVGTYNYVSWYQQHPGKAPKLMIFDVSNRPSGVSDRFSGSKSGNTASLTISGLQAEDEADYYCSSFTTSSTVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 73 - DNA, 369 bp, synthetic construct
  source 1..369: mol_type = other DNA
caggtgcagctggtgcagtctggggctgaggtgaagaagcctggggcctcagtgaaggtctcctgcaaggcttctggatacatcttcaccggctactatatgcactgggtgcgacaggcccctggacaggggcttgagtggatgggatggatcaaccctaacagtggtggcgcaaactatgcacagaagtttcagggcagggtcaccctgaccagggacacgtccatcaccacagtctacatggaactgagcaggctgagatttgacgacacggccgtgtattactgtgcgagaggatcccggtatgactggaaccagaacaactggttcgacccctggggccagggaaccctggtcaccgtctcctca

SEQ ID NO: 74 - DNA, 24 bp, synthetic construct
  source 1..24: mol_type = other DNA
ggatacatcttcaccggctactat

SEQ ID NO: 75 - DNA, 24 bp, synthetic construct
  source 1..24: mol_type = other DNA
atcaaccctaacagtggtggcgca

SEQ ID NO: 76 - DNA, 48 bp, synthetic construct
  source 1..48: mol_type = other DNA
gcgagaggatcccggtatgactggaaccagaacaactggttcgacccc

SEQ ID NO: 77 - DNA, 330 bp, synthetic construct
  source 1..330: mol_type = other DNA
cagtctgccctgactcagcctgcctccgtgtctgggtctcctggacagtcgatcaccatctcctgcactggaaccagcagtgacgttggtacttataactatgtctcctggtaccaacaacacccaggcaaagcccccaaactcatgatttttgatgtcagtaatcggccctcaggggtttctgatcgcttctctggctccaagtctggcaacacggcctccctgaccatctctgggctccaggctgaggacgaggctgattattactgcagctcatttacaaccagcagcactgtggttttcggcggagggaccaagctgaccgtccta

SEQ ID NO: 78 - DNA, 27 bp, synthetic construct
  source 1..27: mol_type = other DNA
agcagtgacgttggtacttataactat

SEQ ID NO: 79 - DNA, 30 bp, synthetic construct
  source 1..30: mol_type = other DNA
agctcatttacaaccagcagcactgtggtt

SEQ ID NO: 80 - DNA, 1362 bp, synthetic construct
  source 1..1362: mol_type = other DNA
caggtgcagctggtgcagtctggggctgaggtgaagaagcctggggcctcagtgaaggtctcctgcaaggcttctggatacatcttcaccggctactatatgcactgggtgcgacaggcccctggacaggggcttgagtggatgggatggatcaaccctaacagtggtggcgcaaactatgcacagaagtttcagggcagggtcaccctgaccagggacacgtccatcaccacagtctacatggaactgagcaggctgagatttgacgacacggccgtgtattactgtgcgagaggatcccggtatgactggaaccagaacaactggttcgacccctggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 81 - DNA, 651 bp, synthetic construct
  source 1..651: mol_type = other DNA
cagtctgccctgactcagcctgcctccgtgtctgggtctcctggacagtcgatcaccatctcctgcactggaaccagcagtgacgttggtacttataactatgtctcctggtaccaacaacacccaggcaaagcccccaaactcatgatttttgatgtcagtaatcggccctcaggggtttctgatcgcttctctggctccaagtctggcaacacggcctccctgaccatctctgggctccaggctgaggacgaggctgattattactgcagctcatttacaaccagcagcactgtggttttcggcggagggaccaagctgaccgtcctaggccagcccaaggccgccccctccgtgaccctgttccccccctcctccgaggagctgcaggccaacaaggccaccctggtgtgcctgatctccgacttctaccccggcgccgtgaccgtggcctggaaggccgactcctcccccgtgaaggccggcgtggagaccaccaccccctccaagcagtccaacaacaagtacgccgcctcctcctacctgtccctgacccccgagcagtggaagtcccaccggtcctactcctgccaggtgacccacgagggctccaccgtggagaagaccgtggcccccaccgagtgctcctga

SEQ ID NO: 82 - protein, 15 aa, synthetic construct
GGGGSGGGGSGGGGS

SEQ ID NO: 83 - protein, 5 aa, synthetic construct
GGGGS

SEQ ID NO: 84 - protein, 10 aa, synthetic construct
GGGGSGGGGS

SEQ ID NO: 85 - protein, 20 aa, synthetic construct
GGGGSGGGGSGGGGSGGGGS

SEQ ID NO: 86 - protein, 25 aa, synthetic construct
GGGGSGGGGSGGGGSGGGGSGGGGS

SEQ ID NO: 87 - protein, 30 aa, synthetic construct
GGGGSGGGGSGGGGSGGGGSGGGGSGGGGS

SEQ ID NO: 88 - protein, 120 aa, synthetic construct
EVQLVESGGGLVQPGGSLRLSCAASGFTFRNYEMNWVRQAPGKGLEWVSYITSSGSTMYYADSVKGRFTISRDNAKNSLYLQMNSLRAEDTAVYYCARDGFYYYYAMDVWGQGTTVTVSS

SEQ ID NO: 89 - protein, 8 aa, synthetic construct
GFTFRNYE

SEQ ID NO: 90 - protein, 8 aa, synthetic construct
ITSSGSTM

SEQ ID NO: 91 - protein, 13 aa, synthetic construct
ARDGFYYYYAMDV

SEQ ID NO: 92 - protein, 111 aa, synthetic construct
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSVGRVFGTGTKVTVL

SEQ ID NO: 93 - protein, 8 aa, synthetic construct
SSNIGNNY

SEQ ID NO: 94 - skipped

SEQ ID NO: 95 - protein, 12 aa, synthetic construct
GTWDSSLSVGRV

SEQ ID NO: 96 - protein, 116 aa, synthetic construct
EVQLVESGGGLVQPGGSLRLSCAASGFTVSSNYMNWVRQAPGKGLEWVSVIYSGGSTFYADSVKGRFTISRHNSKNTLYLQMNSLRPEDTAVYYCARDLGGYFDYWGQGTLVTVSS

SEQ ID NO: 97 - protein, 8 aa, synthetic construct
GFTVSSNY

SEQ ID NO: 98 - protein, 7 aa, synthetic construct
IYSGGST

SEQ ID NO: 99 - protein, 10 aa, synthetic construct
ARDLGGYFDY

SEQ ID NO: 100 - protein, 106 aa, synthetic construct
DIQMTQSPSTLSASVGDRVTITCRASQSFSSWLAWYQQKPGKAPKLLIYKASSLESGVPSRFSGSGSGTEFTLTISSLQPDDFATYYCQQYNSYYTFGQGTKLEIK

SEQ ID NO: 101 - protein, 6 aa, synthetic construct
QSFSSW

SEQ ID NO: 102 - skipped

SEQ ID NO: 103 - protein, 8 aa, synthetic construct
QQYNSYYT

SEQ ID NO: 104 - protein, 117 aa, synthetic construct
EVQLVESGGGLVQPGGSLRLSCAASGLIVSRNYMIWVRQAPGKGLEWVSVIYSGGSTFYADSVKGRFTISRHNSKNTLYLQMNSLRAEDTAVYYCARDLGTGGMDVWGQGTTVTVSS

SEQ ID NO: 105 - protein, 8 aa, synthetic construct
GLIVSRNY

SEQ ID NO: 106 - protein, 11 aa, synthetic construct
ARDLGTGGMDV

SEQ ID NO: 107 - protein, 107 aa, synthetic construct
DIQLTQSPSFLSASVGDRVTITCWASQGISSYLAWYQQKPGKAPKLLIYAASTLQSGVPSRFSGSGSGTEFTLTISSLQPEDFATYYCQQLNSYPLTFGGGTKVEIK

SEQ ID NO: 108 - protein, 6 aa, synthetic construct
QGISSY

SEQ ID NO: 109 - skipped

SEQ ID NO: 110 - protein, 9 aa, synthetic construct
QQLNSYPLT

SEQ ID NO: 111 - protein, 120 aa, synthetic construct
QVQLVESGGGVVQPGRSLRLSCAASGFTFSYYGMHWVRQAPGKGLEWVAVIWYDGSNKFYADSVKGRFTISRDNSKNTLYLQMNSLRAEDTAVYYCAAARPGYYYGMDVWGQGTTVTVSS

SEQ ID NO: 112 - protein, 8 aa, synthetic construct
GFTFSYYG

SEQ ID NO: 113 - protein, 8 aa, synthetic construct
IWYDGSNK

SEQ ID NO: 114 - protein, 13 aa, synthetic construct
AAARPGYYYGMDV

SEQ ID NO: 115 - protein, 107 aa, synthetic construct
DIQMTQSPSSLSASVGDRVTITCRASQGISNYLAWFQQKPGKAPNSLIYAASSLQRGVPSKFSGSGSGTDFTLTISSLQPEDFATYYCQQYNSYPRTFGQGTKVEIK

SEQ ID NO: 116 - protein, 6 aa, synthetic construct
QGISNY

SEQ ID NO: 117 - protein, 9 aa, synthetic construct
QQYNSYPRT

SEQ ID NO: 118 - protein, 117 aa, synthetic construct
EVQLVESGGGLVQPGGSLRLSCAASGITVSSNYMSWVRQAPGKGLEWVSVIYSGGSTFYADSVKGRFTISRHNSKNTLYLQMNSLRAEDTAVYYCARERNFDAFDIWGQGTMVTVSS

SEQ ID NO: 119 - protein, 8 aa, synthetic construct
GITVSSNY

SEQ ID NO: 120 - protein, 11 aa, synthetic construct
ARERNFDAFDI

SEQ ID NO: 121 - protein, 108 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQANSFPLFTFGPGTKVDIK

SEQ ID NO: 122 - protein, 6 aa, synthetic construct
QGISSW

SEQ ID NO: 123 - protein, 10 aa, synthetic construct
QQANSFPLFT

SEQ ID NO: 124 - protein, 119 aa, synthetic construct
EVQLVESGGGLVQPGGSLRLSCAASGFTFSIYEMNWVRQAPGKGLEWVSYITSSGTTIYYADSVKGRFTISRDNAKNSLYLQMNSLRAEDTAVYYCASSSSSGYYFDYWGQGTLVTVSS

SEQ ID NO: 125 - protein, 8 aa, synthetic construct
GFTFSIYE

SEQ ID NO: 126 - protein, 8 aa, synthetic construct
ITSSGTTI

SEQ ID NO: 127 - protein, 12 aa, synthetic construct
ASSSSSGYYFDY

SEQ ID NO: 128 - protein, 113 aa, synthetic construct
DIVMTQSPDSLAVSLGERATINCKSSQSVLYSSNNKNYLAWYQQKPGQPPNLLIYWASTRESGVPDRFSGSGSGTDFTLTISSLQAEDVAVYYCQQYYSTPPTFGQGTKLEIK

SEQ ID NO: 129 - protein, 12 aa, synthetic construct
QSVLYSSNNKNY

SEQ ID NO: 130 - skipped

SEQ ID NO: 131 - protein, 9 aa, synthetic construct
QQYYSTPPT

SEQ ID NO: 132 - protein, 121 aa, synthetic construct
QVQLVQSGAEVKKPGASVKVSCKASGYTFINYYIHWVRQAPGQGLEWMGWINPNSGGTNYAQKFQGRVTMTRDTSISTAYMELSRLRSDDTAVYYCAIITIFGVVTWFDPWGQGTLVTVSS

SEQ ID NO: 133 - protein, 8 aa, synthetic construct
GYTFINYY

SEQ ID NO: 134 - protein, 8 aa, synthetic construct
INPNSGGT

SEQ ID NO: 135 - protein, 14 aa, synthetic construct
AIITIFGVVTWFDP

SEQ ID NO: 136 - protein, 107 aa, synthetic construct [sequence omitted]

SEQ ID NO: 137 - protein, 6 aa, synthetic construct
QSISSY

SEQ ID NO: 138 - protein, 9 aa, synthetic construct
QQSYSTPLT

SEQ ID NO: 139 - protein, 115 aa, synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARDPLLIDYWGQGTLVTVSS

SEQ ID NO: 140 - protein, 8 aa, synthetic construct
GGSISSYY

SEQ ID NO: 141 - protein, 7 aa, synthetic construct
IYYSGST

SEQ ID NO: 142 - protein, 9 aa, synthetic construct
ARDPLLIDY

SEQ ID NO: 143 - protein, 108 aa, synthetic construct
EIVLTQSPGTLSLSPGERATLSCRASQSVSRSYLAWYQQKPGQAPRLLIYGVSSRATGIPDRFSGSGSGTDFTLTISRLEPEDFAVYYCQQYGSSPQTFGQGTKLEIK

SEQ ID NO: 144 - protein, 7 aa, synthetic construct
QSVSRSY

SEQ ID NO: 145 - skipped

SEQ ID NO: 146 - protein, 9 aa, synthetic construct
QQYGSSPQT

SEQ ID NO: 147 - protein, 125 aa, synthetic construct
EVQLVESGGGLVQPGRSLRLSCAASGFTFDDYAMYWVRQAPGKGLEWVSGISWNSGSIGYADSVKGRFTISRDNAKNSLYLQMNSLRAADTALYYCAKDKDWNSRGYYYYGMDVWGQGTTVTVSS

SEQ ID NO: 148 - protein, 8 aa, synthetic construct
GFTFDDYA

SEQ ID NO: 149 - protein, 8 aa, synthetic construct
ISWNSGSI

SEQ ID NO: 150 - protein, 18 aa, synthetic construct
AKDKDWNSRGYYYYGMDV

SEQ ID NO: 151 - protein, 107 aa, synthetic construct
DVVMTQSPSSLSASVGDRVTITCRASQSISSYLNWYQQKPGKAPKLLIYAASSLQSGVPSRFSGSGSGTDFTLTISSLQPEDFATYYCQQSYITPYTFGQGTKVEIK

SEQ ID NO: 152 - protein, 9 aa, synthetic construct
QQSYITPYT

SEQ ID NO: 153 - protein, 116 aa, synthetic construct
EVQLVESGGGLVQSGGSLRLSCAASGLTVSSNYMSWVRQAPGKGLEWVSVIYSGGSTFYSDSVKGRFTISRHNSKNTLYLQMNSLRAEDTAVYYCARARGDAFDIWGQGTMVTVSS

SEQ ID NO: 154 - protein, 8 aa, synthetic construct
GLTVSSNY

SEQ ID NO: 155 - protein, 10 aa, synthetic construct
ARARGDAFDI

SEQ ID NO: 156 - protein, 108 aa, synthetic construct
DIQMTQSPSSLSASVGDRVTITCQASQGISNYLNWYQQKPGKAPKLLISDASNLETGVPSRFSGSGSGTDFTFTISSLQPEDIATYYCQQYDNLRFFTFGPGTKVDIK

SEQ ID NO: 157 - skipped

SEQ ID NO: 158 - protein, 10 aa, synthetic construct
QQYDNLRFFT

SEQ ID NO: 159-172 - skipped (14 entries)

SEQ ID NO: 173 - protein, 120 aa, synthetic construct
EVQLVESGGGLVQPGGSLRLSCAASGFTVSSNYMSWVRQAPGKGLEWVSLIYSGGSTYYADSVKGRFTISRHNSKNTLYLQMNSLRSEDTAVYYCARVPLGDYYYGMDVWGQGTTVTVSS

SEQ ID NO: 174 - protein, 14 aa, synthetic construct
ARVPLGDYYYGMDV

SEQ ID NO: 175 - protein, 107 aa, synthetic construct
DIQMTQSPSSLSASVGDRVTITCQASQDINKYLNWYQQKPGKAPKLLIYDASNLETGVPSRFSGSGSGTDFTFTISSLQPEDIATYYCQQSDNLPLTFGGGTKVEIK

SEQ ID NO: 176 - protein, 6 aa, synthetic construct
QDINKY

SEQ ID NO: 177 - protein, 9 aa, synthetic construct
QQSDNLPLT

SEQ ID NO: 178 - protein, 124 aa, synthetic construct
QVQLVESGGGVVQPGRSLRLSCAASGFTFRNYGMHWVRQAPGKGLEWVSVIWYDGSNRYYADSVKGRFTISRDTSKNTLYLQMNSLRAEDTAVYYCARDPPITGTTGGDAFDIWGQGTMVTVSS

SEQ ID NO: 179 - protein, 8 aa, synthetic construct
GFTFRNYG

SEQ ID NO: 180 - protein, 8 aa, synthetic construct
IWYDGSNR

SEQ ID NO: 181 - protein, 17 aa, synthetic construct
ARDPPITGTTGGDAFDI

SEQ ID NO: 182 - protein, 107 aa, synthetic construct
DIQMTQSPSSLSASVGDRVTITCRASQSISSYLNWYQQKPGKAPHLLIYAASSLQSGVPSRFSGSGSGTDFTLTISSLQPEDFATYYCQQSYSTPYTFGQGTKLEIK

SEQ ID NO: 183 - protein, 9 aa, synthetic construct
QQSYSTPYT

SEQ ID NO: 184 - protein, 119 aa, synthetic construct
QITLKESGPTLVKPTQTLTLTCTFSGFSLSTNVVGVGWIRQPPGKALEWLALIYWNDDKRYSPSLKSRLTITKDTSKNQVVLTMTNMDPVDTATYYCAHGPTGDYFDYWGQGTLVTVSS

SEQ ID NO: 185 - protein, 10 aa, synthetic construct
GFSLSTNVVG

SEQ ID NO: 186 - protein, 7 aa, synthetic construct
IYWNDDK

SEQ ID NO: 187 - protein, 11 aa, synthetic construct
AHGPTGDYFDY

SEQ ID NO: 188 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQTYSTLMYTFGQGTKLEIK

SEQ ID NO: 189 - protein, 10 aa, synthetic construct
QQTYSTLMYT

SEQ ID NO: 190 - protein, 122 aa, synthetic construct
EVQLVESGGGLVQPGRSLRLSCAASGFTFDDYAMHWVRQAPGKGLEWVSGVSWNSGTIDYADSVKGRFTIIRDNAKNSLYLQMNSLRAEDTALYYCSKDMGRLDYYSGLDVWGQGTTVTVSS

SEQ ID NO: 191 - protein, 8 aa, synthetic construct
VSWNSGTI

SEQ ID NO: 192 - protein, 15 aa, synthetic construct
SKDMGRLDYYSGLDV

SEQ ID NO: 193 - protein, 108 aa, synthetic construct
EIVLTQSPGTLSLSPGERATLSCRASQSVSSSDFAWYQQKPGQAPRLLIYGASSRATGIPDRFSGSGSGTDFTLTISRLEPEDFAVYYCQQYGSSPWTFGQGTKVEIK

SEQ ID NO: 194 - protein, 7 aa, synthetic construct
QSVSSSD

SEQ ID NO: 195 - skipped

SEQ ID NO: 196 - protein, 9 aa, synthetic construct
QQYGSSPWT

SEQ ID NO: 197 - protein, 118 aa, synthetic construct
EVQLVESGGGLVQPGGSLRLSCAVSGFTVSSNYMSWVRQAPGKGLEWVSLIYSGGSTFYADSVKGRFTISRHNSKNTLYLQMNSLRAEDTAVYYCAREGGYSYDYNYWGQGTLVTVSS

SEQ ID NO: 198 - protein, 12 aa, synthetic construct
AREGGYSYDYNY

SEQ ID NO: 199 - protein, 107 aa, synthetic construct
DIQMTQSPSSLSASVGDRVTITCQASQDIRNYLNWYQQKPGKAPKLLIYDASNLETGVPSRFSGIGSGTDFTFTISSLQPEDIATYYCQQYDNLPITFGQGTRLEIK

SEQ ID NO: 200 - protein, 6 aa, synthetic construct
QDIRNY

SEQ ID NO: 201 - protein, 9 aa, synthetic construct
QQYDNLPIT

SEQ ID NO: 202 - protein, 117 aa, synthetic construct
EVQLVESGGGLVQPGGSLRLSCAASGLTVSSNYMNWVRQVPGKGLEWVSVIYSGGSKFYVDSVKGRFTISRHNSKNTLYLQMNSLRPEDTAVYYCTRDAQYYGMDVWGQGTTVTVSS

SEQ ID NO: 203 - protein, 7 aa, synthetic construct
IYSGGSK

SEQ ID NO: 204 - protein, 11 aa, synthetic construct
TRDAQYYGMDV

SEQ ID NO: 205 - protein, 108 aa, synthetic construct
DIQMTQSPSSLSASVGDRVTITCRASQSISTYLHWYQQKPGEAPKLLIYAASSLQSGVPSRFSGSGSGTDFTLTISSLQPEDFATYYCQQSYSTPPEIFGQGTKLEIK

SEQ ID NO: 206 - protein, 6 aa, synthetic construct
QSISTY

SEQ ID NO: 207 - protein, 10 aa, synthetic construct
QQSYSTPPEI

SEQ ID NO: 208 - protein, 118 aa, synthetic construct
EVQLVESGGGLVQPGGSLRLSCAASGITVSSNYMTWVRQAPGKGLEWVSVIYSGGSTFYADSVKGRFTISRHNSKNTLYLQMNNLRAEDTAVYYCVRDLEVRGGMDVWGQGTTVTVSS

SEQ ID NO: 209 - protein, 12 aa, synthetic construct
VRDLEVRGGMDV

SEQ ID NO: 210 - protein, 108 aa, synthetic construct
EIVLTQSPGTLSLSPGERATLSCRASQSFSSSYLAWYQQKPGQAPRLLIYGASSRATGIPDRFSGSGSGTDFTLTISRLEPEDFAVYYCQQYGSSPWTFGQGTKVEIK

SEQ ID NO: 211 - protein, 7 aa, synthetic construct
QSFSSSY

SEQ ID NO: 212 - protein, 119 aa, synthetic construct
EVQLVESGGGLVQPGGSLRLSCAASGITVSSNYMSWVRQAPGKGLEWVSLIYSGGSTYYADSVKGRFTISRDNSKNTLYLQMNSLRVEDTAVYYCARDHGMAAAGYNYWGQGTLVTVSS

SEQ ID NO: 213 - protein, 13 aa, synthetic construct
ARDHGMAAAGYNY

SEQ ID NO: 214 - protein, 107 aa, synthetic construct
DIQMTQSPSSLSASVGDRVTITCQASQDINKYLNWYQQKPGKAPKLLIYDASNLETGVPSRFSGSGSGTDFTFTINSLQPEDFATYFCQQYDNLPPAFGQGTKVEIK

SEQ ID NO: 215 - protein, 9 aa, synthetic construct
QQYDNLPPA

SEQ ID NO: 216 - protein, 115 aa, synthetic construct
EVQLVESGGGLVQPGGSLRVSCAASGFTVSRNYMSWVRQAPGKGLEWVSVIYSGGSTFYADSVKGRFTISRDNSKNTLYLQMNSLRAEDTAIYYCARYIPRFDPWGQGTLVTVSS

SEQ ID NO: 217 - protein, 8 aa, synthetic construct
GFTVSRNY

SEQ ID NO: 218 - protein, 9 aa, synthetic construct
ARYIPRFDP

SEQ ID NO: 219 - protein, 107 aa, synthetic construct
DIQMTQSPSSLSASVGDRVTITCQASQDISNYLNWFQQKPGRAPKLLIYDASNLETGVPSRFSGSGSGTDFTFTISSLQPEDIATYYCQQYDNLPITFGQGTRLEIK

SEQ ID NO: 220 - protein, 6 aa, synthetic construct
QDISNY

SEQ ID NO: 221 - protein, 117 aa, synthetic construct
EVQLVESGGGLVQPGGSLRLSCAASGLTVSSNYMSWVRQAPGKGLEWVSVIYSGGSTFYADSVKGRFTISRDNSKNTLYLQMNSLRAEDTAVYYCARDLIKYGMDVWGQGTTVTVSS

SEQ ID NO: 222 - protein, 11 aa, synthetic construct
ARDLIKYGMDV

SEQ ID NO: 223 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLPPHFGPGTKVDIK

SEQ ID NO: 224 - protein, 9 aa, synthetic construct
QQYDNLPPH

SEQ ID NO: 225 - protein, 117 aa, synthetic construct
EVQLVESGGGLVQPGGSLRLSCAASGVTVSSNYMSWVRQAPGKGLEWVSVIYSGGSTFYADSVKGRFTISRDNSKNTLFLQMNSLRIEDTAVYYCARDLGPYGMDVWGQGTTVTVSS

SEQ ID NO: 226 - protein, 8 aa, synthetic construct
GVTVSSNY

SEQ ID NO: 227 - protein, 11 aa, synthetic construct
ARDLGPYGMDV

SEQ ID NO: 228 - protein, 108 aa, synthetic construct
DIQLTQSPSFLSASVGDRVTITCRASQGISSYLAWYQQKPGKAPKLLIYAASTLQSGVPSRFSGSGSGTQFILTISSLQPEDFATYYCQQLNSYPGFTFGPGTKVDIK

SEQ ID NO: 229 - protein, 10 aa, synthetic construct
QQLNSYPGFT

SEQ ID NO: 230 - protein, 118 aa, synthetic construct
QVQLQESGPGLVKPSETLSLTCTVSGGSINNYYWIWIRQPPGKGLEWIGYIYHSGSTNYNPSLKSRVTISVDTSKNQFSLKVSSVTAADTAVYYCANMVRGVYEDDYWGQGTLVTVSS

SEQ ID NO: 231 - protein, 8 aa, synthetic construct
GGSINNYY

SEQ ID NO: 232 - protein, 7 aa, synthetic construct
IYHSGST

SEQ ID NO: 233 - protein, 12 aa, synthetic construct
ANMVRGVYEDDY

SEQ ID NO: 234 - protein, 107 aa, synthetic construct
DIQMTQSPSSLSASVGDRVTITCRASQSISNYLNWYQQKPGKAPKLLIFAASSLRSGVPSRFSGSGSGTDFTLTISSLHPEDFATYYCQQNYNTPLTFGPGTKVDIK

SEQ ID NO: 235 - protein, 6 aa, synthetic construct
QSISNY

SEQ ID NO: 236 - protein, 9 aa, synthetic construct
QQNYNTPLT

SEQ ID NO: 237 - protein, 122 aa, synthetic construct
EVQLVESGGGLIQPGGSLRLSCAASVFTVSYNYMSWVRQAPGKGLEWVSVIYSGGSTYYADSVKGRFTISRDNSKNTLYLQMNSLRAEDTAVYYCARALPGWGGSFEYFDYWGQGTLVTVSS

SEQ ID NO: 238 - protein, 8 aa, synthetic construct
VFTVSYNY

SEQ ID NO: 239 - protein, 16 aa, synthetic construct
ARALPGWGGSFEYFDY

SEQ ID NO: 240 - protein, 107 aa, synthetic construct
DIQMTQSPSTLSASVGDRVTITCRASQSISSWLAWYQQKPGKAPKLLIYKASSLESGVSSRFSGSGSGTEFTLTISSLQPDDFATYYCQQYNSYSVTFGQGTKVEIK

SEQ ID NO: 241 - protein, 6 aa, synthetic construct
QSISSW

SEQ ID NO: 242 - protein, 9 aa, synthetic construct
QQYNSYSVT

SEQ ID NO: 243 - protein, 121 aa, synthetic construct
EVQLVESGGGLVQPGGSLRLSCAASGLTVSSNYMSWVRQAPGKGLEWVSVIYSGGSTFYADSVKGRFTISRDNSKNTLYLQMNSLRPEDTAVYYCAREGRIAATGYGMDVWGQGTTVTVSS

SEQ ID NO: 244 - protein, 15 aa, synthetic construct
AREGRIAATGYGMDV

SEQ ID NO: 245 - protein, 107 aa, synthetic construct
DIQMTQSPSSLSASVGDRVTITCQASQDISNYLNWYQQKPGKAPKLLIYDASNLGTGVPSRFSGSRSGTDFTFTISSLQPEDIATYYCHQYDNLPQTFGPGTKVDIK

SEQ ID NO: 246 - protein, 9 aa, synthetic construct
HQYDNLPQT

SEQ ID NO: 247 - protein, 114 aa, synthetic construct
EVQLVESGGGLVQPGGSLRLSCAASGFTVSSNYMSWVRQAPGKGLEWVSVIYPGGSTFYADSVKGRFTISRDNSKNTLYLQMNSLRAEDTAVYYCARDQRDFAWGQGTLVTVSS

SEQ ID NO: 248 - protein, 7 aa, synthetic construct
IYPGGST

SEQ ID NO: 249 - protein, 8 aa, synthetic construct
ARDQRDFA

SEQ ID NO: 250 - protein, 107 aa, synthetic construct
DIQMTQSPSSLSASVGDRVTITCQASQDISNYLNWYQQKPGKAPKLLIYDASNLETGVPSRFSGSGSGTDFTFTISGLQPEDIATYYCQQYDNLPPTFGQGTRLEIK

SEQ ID NO: 251 - protein, 9 aa, synthetic construct
QQYDNLPPT

SEQ ID NO: 252 - protein, 127 aa, synthetic construct
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARDGRTITMVRGVIDDAFDIWGQGTMVTVSS

SEQ ID NO: 253 - protein, 8 aa, synthetic construct
GFTFSSYA

SEQ ID NO: 254 - protein, 8 aa, synthetic construct
ISYDGSNK

SEQ ID NO: 255 - protein, 20 aa, synthetic construct
ARDGRTITMVRGVIDDAFDI

SEQ ID NO: 256 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGQGTRLEIK

SEQ ID NO: 257 - protein, 120 aa, synthetic construct
EVQLVESGGGLVQPGGSLRLSCAASGFTVSSNYMSWVRQAPGKGLEWVSVIYSGGSTYYADSVKGRFTISRDNSKNTLYLQMNRLRADDTAVYYCVRDPVGRYYYGMDVWGQGTTVTVSS

SEQ ID NO: 258 - protein, 14 aa, synthetic construct
VRDPVGRYYYGMDV

SEQ ID NO: 259 - protein, 107 aa, synthetic construct
DIQMTQSPSSLSASVGDRVTITCQASQDINNYLNWYQQKPGKAPKLLIYDASNLETGVPSRFSGSGSGTDFTFTISSLQPEDIATYYCQQYDNLPRTFGGGTKVEIK

SEQ ID NO: 260 - protein, 6 aa, synthetic construct
QDINNY

SEQ ID NO: 261 - protein, 9 aa, synthetic construct
QQYDNLPRT

SEQ ID NO: 262 - protein, 117 aa, synthetic construct
EVQLVESGGGLIQPGGSLRLSCAASGVTVSSNYMSWVRQAPGKGLEWVSVIYSGGSTFYSDSVKGRFSISRDNSKNTLYLQMNSLRAEDTAVYYCARDLYYYGMDVWGQGTTVTVSS

SEQ ID NO: 263 - protein, 11 aa, synthetic construct
ARDLYYYGMDV

SEQ ID NO: 264 - protein, 108 aa, synthetic construct
DIQLTQSPSFLSASVGDRVTITCRASQGISTYLAWYQQKPGKAPKLLIYAASTLQSGVPSRFSGSGSGTEFTLTISSLQPEDFATYYCQQLDSYPGLTFGGGTKVEIK

SEQ ID NO: 265 - protein, 6 aa, synthetic construct
QGISTY

SEQ ID NO: 266 - protein, 10 aa, synthetic construct
QQLDSYPGLT

SEQ ID NO: 267 - protein, 123 aa, synthetic construct
QVQLVQSGAEVKKPGSSVKVSCKASGGTFSSYAITWVRQAPGQGLEWMGRIIPMFGIANYAQKFQGRVTITADKSTSTAYMEVSSLRSEDTAVYYCARTPFYYDSSGYYLDYWGQGTLVTVSS

SEQ ID NO: 268 - protein, 8 aa, synthetic construct
GGTFSSYA

SEQ ID NO: 269 - protein, 8 aa, synthetic construct
IIPMFGIA

SEQ ID NO: 270 - protein, 16 aa, synthetic construct
ARTPFYYDSSGYYLDY

SEQ ID NO: 271 - protein, 108 aa, synthetic construct
EIVLTQSPGTLSLSPGERATLSCRASQSVSSNYLAWYQQSPGQAPRLLIYGASSRATGIPDRFSGSGSGTDFTLTISRLEPEDFAVYYCQQYVRSPRTFGQGTKVEIK

SEQ ID NO: 272 - protein, 7 aa, synthetic construct
QSVSSNY

SEQ ID NO: 273 - protein, 9 aa, synthetic construct
QQYVRSPRT

SEQ ID NO: 274 - protein, 119 aa, synthetic construct
EVQLVESGGGLVQPGGSLRLSCAASGLTVSSNYMSWVRQAPGKGLEWVSVIYSGGTTYYADSVKGRFTISRHNSKNTLSLQMNSLRAEDTAVYYCARGEGANYYGMDVWGQGTTVTVSS

SEQ ID NO: 275 - protein, 7 aa, synthetic construct
IYSGGTT

SEQ ID NO: 276 - protein, 13 aa, synthetic construct
ARGEGANYYGMDV

SEQ ID NO: 277 - protein, 112 aa, synthetic construct
QSVLTQPPSVSGAPGQRVTISCTGSSSNIGTGYDVHWYQQLPGTAPKLLIYGNSNRPSGVPDRFSGSKSGTSASLAITGLQAEDEADYYCQSYDSSLSGYVVFGGGTKLTVL

SEQ ID NO: 278 - protein, 9 aa, synthetic construct
SSNIGTGYD

SEQ ID NO: 279 - skipped

SEQ ID NO: 280 - protein, 12 aa, synthetic construct
QSYDSSLSGYVV

SEQ ID NO: 281 - protein, 119 aa, synthetic construct
EVQLVESGGGLVQPGGSLRLSCAASGLTVNRNYMSWVRQAPGKGLEWVSLIYSGGSTYYADSVKGRFTISRDNSKNTLYLQMNSLRLEDTAVYYCARGELGIPYGMDVWGQGTTVTVSS

SEQ ID NO: 282 - protein, 8 aa, synthetic construct
GLTVNRNY

SEQ ID NO: 283 - protein, 13 aa, synthetic construct
ARGELGIPYGMDV

SEQ ID NO: 284 - protein, 110 aa, synthetic construct
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAGVFGGGTKLTVL

SEQ ID NO: 285 - protein, 11 aa, synthetic construct
GTWDSSLSAGV

SEQ ID NO: 286 - protein, 128 aa, synthetic construct
EVQLVESGGGLVQPGRSLRLSCAASGFTFDDYAMHWVRQAPGKGLEWVSGISWNRGSIGYADSVKGRFTISRDNAKNSLYLQMSSLRAEDTALYYCAKDGERWDSVVVPSARNGMDVWGQGTTVTVSS

SEQ ID NO: 287 - protein, 8 aa, synthetic construct
ISWNRGSI

SEQ ID NO: 288 - protein, 21 aa, synthetic construct
AKDGERWDSVVVPSARNGMDV

SEQ ID NO: 289 - protein, 112 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSYVFGTGTKVTVL

SEQ ID NO: 290 - protein, 9 aa, synthetic construct
SSNIGAGYD

SEQ ID NO: 291 - protein, 12 aa, synthetic construct
QSYDSSLSGSYV

SEQ ID NO: 292 - protein, 119 aa, synthetic construct
EVQLVESGGGLVQPGGSLRLSCAASEFTVSSNYMSWVRQAPGKGLEWVSVIYSGGSTFYADSVKGRFTISRDNSKNTLYLQMNSLRAEDTAVYYCARALPYGDLHFDYWGQGTLVTVSS

SEQ ID NO: 293 - protein, 8 aa, synthetic construct
EFTVSSNY

SEQ ID NO: 294 - protein, 13 aa, synthetic construct
ARALPYGDLHFDY

SEQ ID NO: 295 - protein, 112 aa, synthetic construct
QSVLTQPPSVSGAPGQRVTISCTGSSSNIGTGYDVHWYQQLPGTAPKLLIYGNTNRPSGVPDRFSGSKSGTSASLAITGLQAEDEADYYCQSYDSSLSDSYVFGTGTKVTVL

SEQ ID NO: 296 - skipped

SEQ ID NO: 297 - protein, 12 aa, synthetic construct
QSYDSSLSDSYV

SEQ ID NO: 298-304 - skipped (7 entries)

SEQ ID NO: 305 - protein, 127 aa, synthetic construct
EVQLVESGGGLVQPGGSLRLSCTASGFTFSNYEMNWVRQAPGKGLEWVSYISSRSGSTLHYADSVKGRFTISRDNAKNSLYLQMNSLRAEDTAVYYCARRGDGTSSLIHHYYYMDVWGKGTTVTVSS

SEQ ID NO: 306 - protein, 8 aa, synthetic construct
GFTFSNYE

SEQ ID NO: 307 - protein, 9 aa, synthetic construct
ISSRSGSTL

SEQ ID NO: 308 - protein, 19 aa, synthetic construct
ARRGDGTSSLIHHYYYMDV

SEQ ID NO: 309 - protein, 110 aa, synthetic construct
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGPVFGGGTKLTVL

SEQ ID NO: 310 - protein, 8 aa, synthetic construct
SSNIGSNT

SEQ ID NO: 311 - skipped

SEQ ID NO: 312 - protein, 11 aa, synthetic construct
AAWDDSLNGPV

SEQ ID NO: 313 - protein, 123 aa, synthetic construct; identical to SEQ ID NO: 64

SEQ ID NO: 314 - protein, 8 aa, synthetic construct
GYIFTGYY

SEQ ID NO: 315 - protein, 8 aa, synthetic construct
INPNSGGA

SEQ ID NO: 316 - protein, 16 aa, synthetic construct
ARGSRYDWNQNNWFDP

SEQ ID NO: 317 - protein, 110 aa, synthetic construct; identical to SEQ ID NO: 68

SEQ ID NO: 318 - protein, 9 aa, synthetic construct
SSDVGTYNY

SEQ ID NO: 319 - protein, 10 aa, synthetic construct
SSFTTSSTVV

SEQ ID NO: 320 - protein, 117 aa, synthetic construct
EVQMVESGGGLVQPGGSLRLSCAASGFTVSSNYMTWVRQAPGKGLEWVSVIYSGGSTYYADSVKGRFTISRHNSKNTLYLQMNSLRAEDTAVYYCARDGVSYGMDVWGQGTTVTVSS

SEQ ID NO: 321 - protein, 11 aa, synthetic construct
ARDGVSYGMDV

SEQ ID NO: 322 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 323 - protein, 10 aa, synthetic construct
QQSYSTPPIT

SEQ ID NO: 324 - protein, 119 aa, synthetic construct
EVQLVESGGGLVHPGGSLRLSCAASGLTVSSNYMSWVRQAPGKGLEWVSVIYSGGSTYYADSVKGRFTISRHNSKNTLFLQMNSLRAEDTAVYYCARDRPITGTTLDVWGQGTTVTVSS

SEQ ID NO: 325 - protein, 13 aa, synthetic construct
ARDRPITGTTLDV

SEQ ID NO: 326 - protein, 108 aa, synthetic construct
DIQMTQSPSSLFASVGDRVTITCRASQSISSYLNWYQQKPGKAPKLLIYAASSLQSGVPSRFSGSGSGTDFTLTISSLQPEDFAFYYCQQTYSTPPITFGQGTRLEIK

SEQ ID NO: 327 - protein, 10 aa, synthetic construct
QQTYSTPPIT

SEQ ID NO: 328 - protein, 117 aa, synthetic construct
EVQLVESGGGLVQPGGSLRLSCAASGFTVSSNYMNWVRQAPGKGLEWVSVIYSGGSTFYADSVRGRFTISRHNSKNTLYLQMNSLRAEDTAVYYCARDGSAYGMDVWGQGTTVTVSS

SEQ ID NO: 329 - protein, 11 aa, synthetic construct
ARDGSAYGMDV

SEQ ID NO: 330-339 - skipped (10 entries)

SEQ ID NO: 340 - protein, 108 aa, synthetic construct
DIQMTQSPSSLSASVGDRVTITCRASQSISSFLNWYQQKPGKAPKLLIYAASSLQSGVPSRFSGSGSGTDFTLTISSLQPEDFATYYCQQSYSSPPITFGQGTRLEIK

SEQ ID NO: 341 - protein, 6 aa, synthetic construct
QSISSF

SEQ ID NO: 342 - protein, 10 aa, synthetic construct
QQSYSSPPIT

SEQ ID NO: 343 - protein, 115 aa, synthetic construct
EVQLVESGGGLVQPGGSLRLSCAASGFTVSSNYMSWVRQAPGKGLEWVSVIYSGGSTFYADSVKGRFTISRHNSKNTLYLQMNSLRAEDTAVYYCAREGYGMDVWGQGTTVTVSS

SEQ ID NO: 344 - protein, 9 aa, synthetic construct
AREGYGMDV

SEQ ID NO: 345 - protein, 120 aa, synthetic construct
QVQLVESGGGVVQPGRSLRLSCVASGFTFSIYGMDWVRQAPGKGLEWVAVISYDGSNKYYADSVKGRFTISRDNSKNTLYLQMNSLRPEDTAVYYCAKDREAAADAFDIWGQGTMVTVSS

SEQ ID NO: 346 - protein, 8 aa, synthetic construct
GFTFSIYG

SEQ ID NO: 347 - protein, 13 aa, synthetic construct
AKDREAAADAFDI

SEQ ID NO: 348 - protein, 107 aa, synthetic construct
DIQMTQSPSSVSASVGDRVTITCRASQGISSWLAWYQQKPGKAPKLLIYAASSLHSGVPSRFSGSGSGTDFTLTISSLQPEDFATYYCQQANSFRLTFGGGTKVEIK

SEQ ID NO: 349 - protein, 9 aa, synthetic construct
QQANSFRLT

SEQ ID NO: 350 - protein, 116 aa, synthetic construct
EVQLVESGGGLVQPGGSLRLSCAASGFIVSRNYMSWVRQAPGKGLEWVSVIYSGGSTFYADSVKGRFTISRHNSKNTLYLQMNSLKAEDTAVYYCARELRGYFDYWGQGTLVTVSS

SEQ ID NO: 351 - protein, 8 aa, synthetic construct
GFIVSRNY

SEQ ID NO: 352 - protein, 10 aa, synthetic construct
ARELRGYFDY

SEQ ID NO: 353 - protein, 107 aa, synthetic construct
DIQLTQSPSFLSASVGDRVTITCWASQGISSYLAWYQQKPGKAPKLLIYAASTLQSGVPSRFSGSGSGTEFTLTISSLQPEDFATYYCQLLNSYPYTFGQGTKLEIK

SEQ ID NO: 354 - protein, 9 aa, synthetic construct
QLLNSYPYT

SEQ ID NO: 355 - protein, 117 aa, synthetic construct
EVQLVESGGGLVQPGGSLRLSCAASGFTVSSNYMTWVRQAPGKGLEWVSVIYSGGSTFYADSVKGRFTISRHNSENTLYLQMNSLRAEDTAVYYCARDLAAAGTDYWGQGALVTVSS

SEQ ID NO: 356 - protein, 11 aa, synthetic construct
ARDLAAAGTDY

SEQ ID NO: 357 - protein, 107 aa, synthetic construct
DIQLTQSPSFLSASVGDRVTITCWASQGISSYLAWYQQKPGKAPKLLIYAASTLQSGVPSRFSGSGSGTEFTLTISSLQPDDFATYYCQHLNSYLYTFGQGTKLEIK

SEQ ID NO: 358 - protein, 9 aa, synthetic construct
QHLNSYLYT

SEQ ID NO: 359 - protein, 120 aa, synthetic construct
QVQLVESGGGVVQPGRSLRLSCAASGFTFSSYGMHWVRQAPGKGLEWVAVISYDGSYKYYADSVKGRFTISRDNSKNTLYLQMNSLRAEDTAVYYCAIDGGTVTTIFDYWGQGTLVTVSS

SEQ ID NO: 360 - protein, 8 aa, synthetic construct
GFTFSSYG

SEQ ID NO: 361 - protein, 8 aa, synthetic construct
ISYDGSYK

SEQ ID NO: 362 - protein, 13 aa, synthetic construct
AIDGGTVTTIFDY

SEQ ID NO: 363 - protein, 108 aa, synthetic construct
DIQMTQSPSSLSASVGDRVTITCRASQSISSYLNWYQQKPGKAPKLLIYTASSLQSGVPSRFSGSGSGTDFTLTISSLQPEDFATYYCQQSYSTPPWTFGQGTKVEIK

SEQ ID NO: 364 - skipped

SEQ ID NO: 365 - protein, 10 aa, synthetic construct
QQSYSTPPWT

SEQ ID NO: 366 - protein, 125 aa, synthetic construct
EVQLVETGGGLIQPGESLRLSCAASGFIVSTNYMNWVRQAPGKGLEWVSVIYRGGSTYYADSVKGRFTISRDNSKNTLFLQMNSLRAEDTAVYYCARGHEDFDYDTPASGAFDIWGQGTMVTVSS

SEQ ID NO: 367 - protein, 8 aa, synthetic construct
GFIVSTNY

SEQ ID NO: 368 - protein, 7 aa, synthetic construct
IYRGGST

SEQ ID NO: 369 - protein, 19 aa, synthetic construct
ARGHEDFDYDTPASGAFDI

SEQ ID NO: 370 - protein, 110 aa, synthetic construct
QSALTQPPSASGSPGQSVTISCTGTSSDVGGHNYVSWYQQHPGKAPKVMIYEVSKRPSGVPDRFSGSKSGNTASLTVSGLQAEDEADYYCSSYAGSNKFVFGTGTKVTVL

SEQ ID NO: 371 - protein, 9 aa, synthetic construct
SSDVGGHNY

SEQ ID NO: 372 - skipped

SEQ ID NO: 373 - protein, 10 aa, synthetic construct
SSYAGSNKFV

SEQ ID NO: 374 - protein, 123 aa, synthetic construct
EVQLVESGGGLVQPGGSLRLSCAASGFAVSSNYMTWVRQAPGKGLEWVATIYSGDSTYYADSVKGRFTISRHNSKNTLSLQMNSLRADDTAIYYCARLSGVLSGTYNGAFDIWGQGTMVTVSS

SEQ ID NO: 375 - protein, 8 aa, synthetic construct
GFAVSSNY

SEQ ID NO: 376 - protein, 7 aa, synthetic construct
IYSGDST

SEQ ID NO: 377 - protein, 17 aa, synthetic construct
ARLSGVLSGTYNGAFDI

SEQ ID NO: 378 - protein, 110 aa, synthetic construct
QSALTQPASVSGSPGQSITISCTGTSSDFGNYNIVSWYQQHPGQAPKVMIYEGNKRPSGVSNRFSGSKSGHTASLTISGLQAEDEADYYCSSYAGSSTWVFGGGTKLTVL

SEQ ID NO: 379 - protein, 9 aa, synthetic construct
SSDFGNYNI

SEQ ID NO: 380 - skipped

SEQ ID NO: 381 - protein, 10 aa, synthetic construct
SSYAGSSTWV

SEQ ID NO: 382 - protein, 122 aa, synthetic construct
EVQLVESGGGLVQPGGSLRLSCAASGFTVSSNYMSWVRQAPGKGLEWVSAIYSGDSTYYADSVKGRFTISKDNSKNTLYLQMNSLRGEDTAVYYCARLGGALQAYDGAFDYWGQGTLATVSS

SEQ ID NO: 383 - protein, 8 aa, synthetic construct
GFTVSSNY

SEQ ID NO: 384 - protein, 16 aa, synthetic construct
ARLGGALQAYDGAFDY

SEQ ID NO: 385 - protein, 110 aa, synthetic construct
QSALTQPASVSGSPGQSITISCTGTSSDFGSYNLVSWYQQHPGKAPKLMIYEGNKRPSGVSIRFSGSKSGNTASLTISGLQAEDEADYYCSSYAGSSTWVFGGGTKLTVL

SEQ ID NO: 386 - protein, 9 aa, synthetic construct
SSDFGSYNL

SEQ ID NO: 387 - protein, 121 aa, synthetic construct
EVQLVESGGGLVQPGGSLRLSCAASGFTVTSNYMSWVRQAPGKGLEWVSAIYSGESTYYADSVKGRFTISRDSSKNTLYLQMNSLRVEDTAVYYCASIHDYSNYVGGFDPWGQGTLVTVSS

SEQ ID NO: 388 - protein, 8 aa, synthetic construct
GFTVTSNY

SEQ ID NO: 389 - protein, 7 aa, synthetic construct
IYSGEST

SEQ ID NO: 390 - protein, 15 aa, synthetic construct
ASIHDYSNYVGGFDP

SEQ ID NO: 391 - protein, 110 aa, synthetic construct
QSALTQPASVSGSPGQSITISCTGPSSDFGSYNLVSWYQQHPGKAPKLMIYEGTKRPSGVSNRFSGSKSGNTASLTISGLQAEDEADYYCSSYAGSSTWVFGGGTKLTVL

SEQ ID NO: 392 - skipped

SEQ ID NO: 393 - protein, 122 aa, synthetic construct
QVQLQESGPGLVKPSETLSLTCTVSGGSISGYYWSWIRQPPGKGLEWIGYIYYSGSTNYNPSLKSRVTMSVDTSKNQFSLKLSSVTAADTAVYYCARYPLYSESSGYHFDYWGHGTLVTVSS

SEQ ID NO: 394 - protein, 8 aa, synthetic construct
GGSISGYY

SEQ ID NO: 395 - protein, 7 aa, synthetic construct
IYYSGST

SEQ ID NO: 396 - protein, 16 aa, synthetic construct
ARYPLYSESSGYHFDY

SEQ ID NO: 397 - protein, 110 aa, synthetic construct
QSALTQPASVSGSPGQSITISCTGTSSDVGSYNLVSWYQQDPGKAPKLMLYEDSKRPSGVSNRFSGSKSGNTASLTISGLQAEDEADYYCSSYAGRSTWVFGGGTKLTVL

SEQ ID NO: 398 - protein, 9 aa, synthetic construct
SSDVGSYNL

SEQ ID NO: 399 - skipped

SEQ ID NO: 400 - protein, 10 aa, synthetic construct
SSYAGRSTWV

SEQ ID NO: 401 - protein, 117 aa, synthetic construct
EVQLVESGGGLVQPGGSLRLSCAATGITVSSNYMSWVRQAPGKGLEWVSLIYSGGSTFYADSVKGRFTISRHNSKNTLYLQMSSLRAEDTAVYYCARDLVDFGMDVWGQGTTVTVSS

SEQ ID NO: 402 - protein, 8 aa, synthetic construct
GITVSSNY

SEQ ID NO: 403 - protein, 7 aa, synthetic construct
IYSGGST

SEQ ID NO: 404 - protein, 11 aa, synthetic construct
ARDLVDFGMDV

SEQ ID NO: 405 - protein, 107 aa, synthetic construct
DIQLTQSPSSLSASVGDRVTITCRASQGISNYLAWYQQKPGKAPKLLIYAASTLQSGVPSRFSGSGSGTDFTLTISSLQPEDFGTYYCQQLNSDSPTFGQGTKVEIK

SEQ ID NO: 406 - protein, 6 aa, synthetic construct
QGISNY

SEQ ID NO: 407 - skipped

SEQ ID NO: 408 - protein, 9 aa, synthetic construct
QQLNSDSPT

SEQ ID NO: 409 - protein, 128 aa, synthetic construct
EVQLVQSGAEVKKPGESLKISCKGSGYSFTSYWIGWVRQMPGKGLEWMGIIYPGDTNTRYSPSFQGQVTISADKSISTAYLQWSSLKASDTAMYYCARLRAPSGPWEGAIPGGGMDVWGQGTTVTVSS

SEQ ID NO: 410 - protein, 8 aa, synthetic construct
GYSFTSYW

SEQ ID NO: 411 - protein, 8 aa, synthetic construct
IYPGDTNT

SEQ ID NO: 412 - protein, 21 aa, synthetic construct
ARLRAPSGPWEGAIPGGGMDV

SEQ ID NO: 413 - protein, 108 aa, synthetic construct
EIVMTQSPATLSVSPGERATLSCRASQSVSSNLAWYQQKHGQAPRLLIYGASTRATGIPARFSGSGSGTEFTLTISSLQSEDFAVYYCQQYNNWPPFTFGPGTKVDIK

SEQ ID NO: 414 - protein, 6 aa, synthetic construct
QSVSSN

SEQ ID NO: 415-416 - skipped (2 entries)

SEQ ID NO: 417 - protein, 10 aa, synthetic construct
QQYNNWPPFT

SEQ ID NO: 418 - protein, 127 aa, synthetic construct
QVQLVQSGAEVKKPGASVKVSCKASGYTFTDYYMHWVRQAPGQGLEWMGWINPNSGGTNYAQKFQGRVTMTRDTSISTAYMELSRLRSDDTAIYYCARTLFKYYDILIGYGEYFQHWGQGTLVIVSS

SEQ ID NO: 419 - protein, 8 aa, synthetic construct
GYTFTDYY

SEQ ID NO: 420 - protein, 8 aa, synthetic construct
INPNSGGT

SEQ ID NO: 421 - protein, 20 aa, synthetic construct
ARTLFKYYDILIGYGEYFQH

SEQ ID NO: 422 - protein, 108 aa, synthetic construct
EIVLTQSPGTLSLSPGERATLSCRASQSVSSSYLAWYQQKPGQAPRLLIYGASSRATAIPDRFSGSGSGTDFTLTISRLEPEDFAVYFCQQYGSSPWTFGQGTKVEIK

SEQ ID NO: 423 - protein, 7 aa, synthetic construct
QSVSSSY

SEQ ID NO: 424 - protein, 9 aa, synthetic construct
QQYGSSPWT

SEQ ID NO: 425 - protein, 120 aa, synthetic construct
EVQLVESGGGLVQPGGSLRLSCAASGFTVSRSYMTWVRQAPGKGLEWVSVIFTGGRTDHADSVKGRFTISRHNSKNTLYLQMNSLRTEDTAVYYCARVAIVGATGAFDIWGQGTMVTVSS

SEQ ID NO: 426 - protein, 8 aa, synthetic construct
GFTVSRSY

SEQ ID NO: 427 - protein, 7 aa, synthetic construct
IFTGGRT

SEQ ID NO: 428 - protein, 14 aa, synthetic construct
ARVAIVGATGAFDI

SEQ ID NO: 429 - protein, 112 aa, synthetic construct
QSVLTQPPSVSGAPGQRVTISCTGSSSNIGAGDDVHWYQQLPGTAPKLLIYGNSNRPSGVPDRFSGSKSGTSASLAITGLQAEDEADYYCQSYDSSLSGSVVFGGGTKLTVL

SEQ ID NO: 430 - protein, 9 aa, synthetic construct
SSNIGAGDD

SEQ ID NO: 431 - skipped

SEQ ID NO: 432 - protein, 12 aa, synthetic construct
QSYDSSLSGSVV

SEQ ID NO: 433 - protein, 123 aa, synthetic construct
EVQLVESGGGLVQPGRSLRLSCVASGFTFDDSAMHWVRQPPGKGLEWVSGISWNSGIIGYAESVKGRFIISRDNAKNSLFLHMRGLRPEDTAVYYCARDRLELSYYYYGMDVWGQGTTVTVSS

SEQ ID NO: 434 - protein, 8 aa, synthetic construct
GFTFDDSA

SEQ ID NO: 435 - protein, 8 aa, synthetic construct
ISWNSGII

SEQ ID NO: 436 - protein, 16 aa, synthetic construct
ARDRLELSYYYYGMDV

SEQ ID NO: 437 - protein, 110 aa, synthetic construct
QSVLTQPPSASGTPGQRVTISCSGSSSNIGSNTVNWYQQFPGTAPRLLIYSNDQRPSGIPDRFSGSKSGTSASLAISGLQSEDEADYYCSTWDDSLNGAVFGGGTQLTVL

SEQ ID NO: 438 - protein, 8 aa, synthetic construct
SSNIGSNT

SEQ ID NO: 439 - skipped

SEQ ID NO: 440 - protein, 11 aa, synthetic construct
STWDDSLNGAV

SEQ ID NO: 441 - protein, 121 aa, synthetic construct
RVTLRESGPALVKPTQTLTLTCTFSGFSLYSSGMSVSWIRQPPGKALEWLARIDWDDDEYYSSFLKTRLTISKDISKNQVVLTMTNLDPVDTATYYCVRPRYSSNFGFEYWGQGTLVTVSS

SEQ ID NO: 442 - protein, 10 aa, synthetic construct
GFSLYSSGMS

SEQ ID NO: 443 - protein, 7 aa, synthetic construct
IDWDDDE

SEQ ID NO: 444 - protein, 13 aa, synthetic construct
VRPRYSSNFGFEY

SEQ ID NO: 445 - protein, 110 aa, synthetic construct
QSVLTQPPSVSAAPGQKVTISCSGSISNIGNNYVSWYQQLPGTAPKLLIYDNDKRPSGIPDRFSGSRSGTSATLGITGLQTGDEADYYCGTWDSSLTAYVFGTGTKVTVL

SEQ ID NO: 446 - protein, 8 aa, synthetic construct
ISNIGNNY

SEQ ID NO: 447 - skipped

SEQ ID NO: 448 - protein, 11 aa, synthetic construct
GTWDSSLTAYV

SEQ ID NO: 449 - protein, 121 aa, synthetic construct
QVQLVQSGAEVKKPGSSVKVSCKASGDTFSTYAINWVRQAPGQGLEWMGRFIHIFGTANYAQKFQGRVTITADESTSTAYMELRSLRSEDTAVYYCARDGVDYGDYRPDYWGQGTLVTVSS

SEQ ID NO: 450 - protein, 8 aa, synthetic construct
GDTFSTYA

SEQ ID NO: 451 - protein, 8 aa, synthetic construct
FIHIFGTA

SEQ ID NO: 452 - protein, 14 aa, synthetic construct
ARDGVDYGDYRPDY

SEQ ID NO: 453 - protein, 108 aa, synthetic construct
EIVLTQSPGTLSLSPGERATLSCRASQSVSSNYLAWYQQKPGQAPRLLIYGASSRATGIPERFSGSGSGTDFTLTISRLEPEDFAVYYCQQYGSSLYTFGQGTKLEIK

SEQ ID NO: 454 - protein, 7 aa, synthetic construct
QSVSSNY

SEQ ID NO: 455 - protein, 9 aa, synthetic construct
QQYGSSLYT

SEQ ID NO: 456 - protein, 119 aa, synthetic construct
QVQLVQSGAEVKKPGSSVKVSCKAPGGTFSTYAINWVRQAPGQGLEWMGRIVPFFGTTNYAQKFQGRVTITADESTSTAYMELSSLTSEDTAVYYCARGPRPFGELLYWGQGTLVTVSS

SEQ ID NO: 457 - protein, 8 aa, synthetic construct
GGTFSTYA

SEQ ID NO: 458 - protein, 8 aa, synthetic construct
IVPFFGTT

SEQ ID NO: 459 - protein, 12 aa, synthetic construct
ARGPRPFGELLY

SEQ ID NO: 460 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQAEDIATYYCQQYDNLLFTFGPGTKVDIK

SEQ ID NO: 461 - protein, 6 aa, synthetic construct
QDISNY

SEQ ID NO: 462 - skipped

SEQ ID NO: 463 - protein, 9 aa, synthetic construct
QQYDNLLFT

SEQ ID NO: 464 - protein, 119 aa, synthetic construct
EVQLVETGGGLIQPGGSLRLSCAAPGFIVSSNYMSWVRQAPGKGLEWVSLLYPGGSTYYADSVKGRFTISRDNSKNTLYLQMNSLRADDTAVYYCARVLPIVEDHFDYWGQGTLVTVSS

SEQ ID NO: 465 - protein, 8 aa, synthetic construct
GFIVSSNY

SEQ ID NO: 466 - protein, 7 aa, synthetic construct
LYPGGST

SEQ ID NO: 467 - protein, 13 aa, synthetic construct
ARVLPIVEDHFDY

SEQ ID NO: 468 - protein, 110 aa, synthetic construct
DIQLTQSPSFLSASVGDRVTITCRASQGMSSYLAWYQKKPGKAPKLLIYGATILQSGVPSRFSGSGSGTEFTLTISSLQPEDFATYYCQQLNSDPLYMYTFGQGTKLEIK

SEQ ID NO: 469 - protein, 6 aa, synthetic construct
QGMSSY

SEQ ID NO: 470 - skipped

SEQ ID NO: 471 - protein, 12 aa, synthetic construct
QQLNSDPLYMYT

SEQ ID NO: 472 - protein, 122 aa, synthetic construct
QVQLVESGGGVVQPGRSLRLSCAASGFVFNSYGMHWVRQAPGKGLEWVAVLWYEGSKNYYADSVKGRFTISRDNSKNTLYLQMNSLRAEDTAVYYCARHGSGSFFGYYLDYWGQGTLVTVSS

SEQ ID NO: 473 - protein, 8 aa, synthetic construct
GFVFNSYG

SEQ ID NO: 474 - protein, 8 aa, synthetic construct
LWYEGSKN

SEQ ID NO: 475 - protein, 15 aa, synthetic construct
ARHGSGSFFGYYLDY

SEQ ID NO: 476 - protein, 107 aa, synthetic construct
DIQMTQSPSSLSASVGDRVTITCRASHNINDFLNWYQQKPGKAPRLLIYAASSLQSGVPSRFSGSGSGTDFTLTISSLQPEDFATYYCQESYTTPPTFGQGTKLEIK

SEQ ID NO: 477 - protein, 6 aa, synthetic construct
HNINDF

SEQ ID NO: 478 - protein, 9 aa, synthetic construct
QESYTTPPT

SEQ ID NO: 479 - protein, 122 aa, synthetic construct
QLQLQESGPGLVKSSETLSLTCTVSASITGSTYYWGWIRQPPGKGLEWIGSIYYSGTTYYKASLKSRVTISVDTSKNQFSLKLTSVTVADTAVYYCVSHLLGNYHKYGMDVWGQGTTVTVSS

SEQ ID NO: 480 - protein, 9 aa, synthetic construct
ASITGSTYY

SEQ ID NO: 481 - protein, 7 aa, synthetic construct
IYYSGTT

SEQ ID NO: 482 - protein, 15 aa, synthetic construct
VSHLLGNYHKYGMDV

SEQ ID NO: 483 - protein, 112 aa, synthetic construct
QSVLTQPPSASGTPGQRVTISCSGSRSNIGINTVNWYQQFPGTALNLLIFSDNQRPSGVPDRFSGSKSGTSASLAISGLQSEDEADYYCAAWDDSLNGPDWVFGGGTKLTVL

SEQ ID NO: 484 - protein, 8 aa, synthetic construct
RSNIGINT

SEQ ID NO: 485 - skipped

SEQ ID NO: 486 - protein, 13 aa, synthetic construct
AAWDDSLNGPDWV

SEQ ID NO: 487 - protein, 123 aa, synthetic construct
EVQLVESGGALVQPGGSLRLSCAASGFPFTIYAMTWVRQAPGKGLEWVSVITGSGDSTYYADSVKGRFTISRDNSKNTLYLQMNSLRAEDTAVYYCVKTGEGFFYYYYGMDVWGQGTTVTVSS

SEQ ID NO: 488 - protein, 8 aa, synthetic construct
GFPFTIYA

SEQ ID NO: 489 - protein, 8 aa, synthetic construct
ITGSGDST

SEQ ID NO: 490 - protein, 16 aa, synthetic construct
VKTGEGFFYYYYGMDV

SEQ ID NO: 491 - protein, 110 aa, synthetic construct
QSVLTQPPSVSAAPGQKVTISCSGSSSNIGNSYISWYQQLPGTAPKLLIYVNNKRPSGIPDRFSGSKSGTSATLGITGLQTGDEADYYCGTWDSSLSAGVFGSGTKVTVL

SEQ ID NO: 492 - protein, 8 aa, synthetic construct
SSNIGNSY

SEQ ID NO: 493 - skipped

SEQ ID NO: 494 - protein, 11 aa, synthetic construct
GTWDSSLSAGV

SEQ ID NO: 495 - protein, 121 aa, synthetic construct
EVQLVESGGGLVQPGGSLRLSCAASGFSFSSNWMSWVRQAPGKGLEWVGNINQDGSEKYYVDSVEGRFTISRDNAKKSLYLQMNSLRAEDTAVYYCARDLGVLWFGDFVDWGQGALVTVSS

SEQ ID NO: 496 - protein, 8 aa, synthetic construct
GFSFSSNW

SEQ ID NO: 497 - protein, 8 aa, synthetic construct
INQDGSEK

SEQ ID NO: 498 - protein, 14 aa, synthetic construct
ARDLGVLWFGDFVD

SEQ ID NO: 499 - protein, 107 aa, synthetic construct
DIQMTQSPSSLSASVGDRVTITCRASQAISNSLAWYQQKPGKAPKLLLYAASTLESGVPSRFSGSGSGTDYTLTISSLQPEDFATYYCLQYYGSPRTFGQGTKVEIK

SEQ ID NO: 500 - protein, 6 aa, synthetic construct
QAISNS

SEQ ID NO: 501 - protein, 9 aa, synthetic construct
LQYYGSPRT

SEQ ID NO: 502 - protein, 11 aa, synthetic construct
ARDLVVYGMDV

SEQ ID NO: 503 - protein, 107 aa, synthetic construct
DIQLTQSPSSLSASVGDRVTITCRASQGISSYLAWYQQKPGKAPKLLIYAASTLQSGVPSRFSGSGSGTDFTLTISSLQPEDFATYYCQHLNSDPHTFGQGTKLEIK

SEQ ID NO: 504 - protein, 6 aa, synthetic construct
QGISSY

SEQ ID NO: 505 - protein, 9 aa, synthetic construct
QHLNSDPHT

SEQ ID NO: 506 - protein, 123 aa, synthetic construct
EVQLVESGGGLVQPGGSLRLSCSASGFTFSRYAMYWVRQAPGKGLEYVSAISSDGGSTYDADSVKGRFTISRANSKNTLYLQMSSLRAEDTAVYYCVKGLRELLYYYYGMDVWGQGTTVTVSS

SEQ ID NO: 507 - protein, 8 aa, synthetic construct
GFTFSRYA

SEQ ID NO: 508 - protein, 8 aa, synthetic construct
ISSDGGST

SEQ ID NO: 509 - protein, 16 aa, synthetic construct
VKGLRELLYYYYGMDV

SEQ ID NO: 510 - protein, 107 aa, synthetic construct
DIQMTQSPSSLSASVGDRVTITCRAGQSISSFLNWYQQKPGKAPKLLIYAASSLQSGVPSRFSGSGSGTDFTLTISSLQPEDFATYYCQQSYITPFTFGPGTKVDIK

SEQ ID NO: 511 - protein, 6 aa, synthetic construct
QSISSF

SEQ ID NO: 512 - protein, 9 aa, synthetic construct
QQSYITPFT

SEQ ID NO: 513 - protein, 120 aa, synthetic construct
EVQLVESGGGLVQPGGSLRLSCAASGFTFSSYEMNWVRQAPGKGLEWVSYISSSGSILYYADSVKGRFTASRDNAKNSLYLQMNSLRAEDTGVYYCARREYQLLRPFEFWGQGTLVTVSS

SEQ ID NO: 514 - protein, 8 aa, synthetic construct
GFTFSSYE

SEQ ID NO: 515 - protein, 8 aa, synthetic construct
ISSSGSIL

SEQ ID NO: 516 - protein, 13 aa, synthetic construct
ARREYQLLRPFEF

SEQ ID NO: 517 - protein, 108 aa, synthetic construct
DIQMTQSPSTLSASVGDRVTITCRASQSISSWLAWYQQKSGKAPKLLIYKASSLESGVPSRFSGSGSGTEFTLTISSLQPDDFATYYCQQYDSYSPWTFGQGTKVEIK

SEQ ID NO: 518 - protein, 6 aa, synthetic construct
QSISSW

SEQ ID NO: 519 - skipped

SEQ ID NO: 520 - protein, 10 aa, synthetic construct
QQYDSYSPWT

SEQ ID NO: 521 - protein, 116 aa, synthetic construct
EVQLVETGGGLIQPGGSLRLSCAASEFIVSRNYMSWVRQAPGKGLEWVSLIYSGGSTYYADSVKGRFTISRDNSKNILYLQMNSLRAEDTAVYYCARDRGGISDYWGQGTLVTVSS

SEQ ID NO: 522 - protein, 8 aa, synthetic construct
EFIVSRNY

SEQ ID NO: 523 - protein, 10 aa, synthetic construct
ARDRGGISDY

SEQ ID NO: 524 - protein, 107 aa, synthetic construct
DIQMTQSPSSLSASVGDRVTITCQASQDINKYLNWFQQKPGKAPKLLIYDASNLETGVPSRFSGSGSGTDFTFTISSLQPEDIATYYCQQYDNLPRTFGQGTKLEIK

SEQ ID NO: 525 - protein, 6 aa, synthetic construct
QDINKY

SEQ ID NO: 526 - protein, 9 aa, synthetic construct
QQYDNLPRT

SEQ ID NO: 527 - protein, 122 aa, synthetic construct
QVQLVQSGAEVKKPGASVRVSCKASGYTFTGNYIHWVRQAPGQGLEWMAWINPNSGGKNYAQKFQGRVTLTRDTSVNTAYMELSRLRSDDTAMYYCARDVNYNILTGPGGLWGQGTLVTVSS

SEQ ID NO: 528 - protein, 8 aa, synthetic construct
GYTFTGNY

SEQ ID NO: 529 - protein, 8 aa, synthetic construct
INPNSGGK

SEQ ID NO: 530 - protein, 15 aa, synthetic construct
ARDVNYNILTGPGGL

SEQ ID NO: 531 - protein, 110 aa, synthetic construct
QSALTQPPSASGSPGQSVTISCTGTSSDVGGYNFVSWYQQHPGKAPKLMIYEVSKRPSGVPDRFSGSKSGNTASLTVSGLQAEDEADYYCSSYAGSNNFVFGTGTKVTVL

SEQ ID NO: 532 - protein, 9 aa, synthetic construct
SSDVGGYNF

SEQ ID NO: 533 - protein, 10 aa, synthetic construct
SSYAGSNNFV

SEQ ID NO: 534 - protein, 122 aa, synthetic construct
QVQLVQSGADVKKPGASVKVSCKTSGYIFTGYYIHWVRQAPGQGLEWMGWINPNSGGTNSAQKFQGRVTMTRDTSISTVYMELSRLRSDDTAVYYCAKSNSFSVLDYNFDYWGQGTLVTVSS

SEQ ID NO: 535 - protein, 8 aa, synthetic construct
GYIFTGYY

SEQ ID NO: 536 - protein, 15 aa, synthetic construct
AKSNSFSVLDYNFDY

SEQ ID NO: 537 - protein, 109 aa, synthetic construct
SSELTQDPAVSVALGQTVRITCQGDSLRSYYASWYQQKPGQAPVLVIYDKNNRPSGIPDRFSGSSSGNTASLTITGAQAEDEADYYCNSRDSSRNHLVVFGGGTKLTVL

SEQ ID NO: 538 - protein, 6 aa, synthetic construct
SLRSYY

SEQ ID NO: 539 - skipped

SEQ ID NO: 540 - protein, 12 aa, synthetic construct
NSRDSSRNHLVV

SEQ ID NO: 541 - protein, 122 aa, synthetic construct
QVQLVQSGAEVKKPGASVKVSCKASGYIFTDHYMHWVRQAPGQGLEWMGWINPNSGATNYAQNFQGRVTMTRDTSIITVYMELSRLRSDDTAVYYCARDSSPYMIRYTMDVWGQGTTITVSS

SEQ ID NO: 542 - protein, 8 aa, synthetic construct
GYIFTDHY

SEQ ID NO: 543 - protein, 8 aa, synthetic construct
INPNSGAT

SEQ ID NO: 544 - protein, 15 aa, synthetic construct
ARDSSPYMIRYTMDV

SEQ ID NO: 545 - protein, 111 aa, synthetic construct
QSALTQPASVSGSPGQSITISCTGTSSDVGSYNLVSWYQQHPGKAPKLMIYELTKRPSGVSNRFSGSKSGNTASLTISGLQAEDEADYYCSSYRGSVTDVVFGGGTKLTVL

SEQ ID NO: 546 - skipped

SEQ ID NO: 547 - protein, 11 aa, synthetic construct
SSYRGSVTDVV

SEQ ID NO: 548 - protein, 118 aa, synthetic construct
QVQLQESGPGLVKPSGTLSLTCDVSGGSISSNNWWSWVRQRPGKGLQWIGEIYHNENTNYNPSLKSRVIMSIDKSKNQFSLKLTSVTAADTAVYYCARGDVLDWFDPWGQGTLVTVSS

SEQ ID NO: 549 - protein, 9 aa, synthetic construct
GGSISSNNW

SEQ ID NO: 550 - protein, 7 aa, synthetic construct
IYHNENT

SEQ ID NO: 551 - protein, 11 aa, synthetic construct
ARGDVLDWFDP

SEQ ID NO: 552 - protein, 110 aa, synthetic construct
QSALTQPASVSGSPGQSTTISCTGTSSDVGGYNYVSWYQQYPGKAPKLMIYDVTNRPSGVSHRFSGSKSGNTASLTISGLQAEDEADYYCSSYTSSSTVIFGGGTKLTVL

SEQ ID NO: 553 - protein, 9 aa, synthetic construct
SSDVGGYNY

SEQ ID NO: 554 - skipped

SEQ ID NO: 555 - protein, 10 aa, synthetic construct
SSYTSSSTVI

SEQ ID NO: 556 - protein, 119 aa, synthetic construct
QITLKESGPTLVKPTQTLTLTCTFSGFSFSTSGVGVGWIRQPPGKTLEWLALIYWDDDKRYSPSLKSRLTITKDTSKNQVVLTMTNMDPVDTATYFCAHHGIPTIFGYWGQGALVTVSS

SEQ ID NO: 557 - protein, 10 aa, synthetic construct
GFSFSTSGVG

SEQ ID NO: 558 - protein, 7 aa, synthetic construct
IYWDDDK

SEQ ID NO: 559 - protein, 11 aa, synthetic construct
AHHGIPTIFGY

SEQ ID NO: 560 - protein, 109 aa, synthetic construct
QSALTQPASVSGSPGQSITISCTGTSSDLGVFNYVSWYQQHPGKAPKLMIYEVTNRPSGVSNRFSGSKSGNTASLTISGLQAEDEADYYCSSYTTSSTVFGGGTKLTVL

SEQ ID NO: 561 - protein, 9 aa, synthetic construct
SSDLGVFNY

SEQ ID NO: 562 - skipped

SEQ ID NO: 563 - protein, 9 aa, synthetic construct
SSYTTSSTV

SEQ ID NO: 564 - protein, 120 aa, synthetic construct
QVQLVQSGAEVKKPGASVKVSCKASGYTFTGYFIHWVRQAPGQGLEWMGWISPISGGTNYAQKFQGRVTVTRDTSITTAYMELSRLTSDDTAVYHCAREGGTWKGDFDYWGQGTLVTVSS

SEQ ID NO: 565 - protein, 8 aa, synthetic construct
GYTFTGYF

SEQ ID NO: 566 - protein, 8 aa, synthetic construct
ISPISGGT

SEQ ID NO: 567 - protein, 13 aa, synthetic construct
AREGGTWKGDFDY

SEQ ID NO: 568 - protein, 110 aa, synthetic construct
QSALTQPASVSGSPGQSITISCTGTSSDVESYNLVSWYQQHPGKAPKLMIYEGSKRPSGVSNRFSGSKSGNTASLTISGLQAEDEADYYCSSYAGSSTWVFGGGTKLTVL

SEQ ID NO: 569 - protein, 9 aa, synthetic construct
SSDVESYNL

SEQ ID NO: 570 - skipped

SEQ ID NO: 571 - protein, 122 aa, synthetic construct
QVQLVQSGAEVKKPGASVKVSCKASGYTFTDYFIHWVRQAPGQGLEWMGWINPTSGGTNYAQKFRGRVTMTRDTSISTAYVELSRLRSDDTALYYCARDLPFYSDTKQFDYWGQGTLVTVSS

SEQ ID NO: 572 - protein, 8 aa, synthetic construct
GYTFTDYF

SEQ ID NO: 573 - protein, 8 aa, synthetic construct
INPTSGGT

SEQ ID NO: 574 - protein, 15 aa, synthetic construct
ARDLPFYSDTKQFDY

SEQ ID NO: 575 - protein, 110 aa, synthetic construct
QSALTQPASVSGYPGQSITISCTGTSSDVGGYNYVSWYQQHPGKAPKLMIFDVSNRPSGVSNRFSGSKSGNTASLTFSGLQAEDEADYYCSSYTSSSTLVFGGGTKLTVL

SEQ ID NO: 576 - skipped

SEQ ID NO: 577 - protein, 10 aa, synthetic construct
SSYTSSSTLV

SEQ ID NO: 578 - protein, 120 aa, synthetic construct
QVQLVQSGAEVKKPGSSVKVSCKASGGTFSNNAISWVRQAPGQGLEWMGKIIPILGIGGDAQKFQGRFTITADKSTSTAYMELSSLRSEDTAVYYCANSGDSRPHAFDIWGQGTMVTVSS

SEQ ID NO: 579 - protein, 8 aa, synthetic construct
GGTFSNNA

SEQ ID NO: 580 - protein, 8 aa, synthetic construct
IIPILGIG

SEQ ID NO: 581 - protein, 13 aa, synthetic construct
ANSGDSRPHAFDI

SEQ ID NO: 582 - protein, 110 aa, synthetic construct
QSALTQPASVSGSPGQSITISCTGTSSDVGSYNLVSWYQQHPGKAPKLMIYEVSKRPSGVSNRFSGSKSGNTASLTISGLQAEDEADYYCSSYAGSRREVFGGGTKLTVL

SEQ ID NO: 583 - protein, 10 aa, synthetic construct
SSYAGSRREV

SEQ ID NO: 584 - protein, 126 aa, synthetic construct
EVQLVESGGGLVQPGGSLRLSCAASGFTLSSYDMHWVRQVIGKGLEWVSTIGTSGDTYYPGSVKGRFTISRENAKNSLYLQMNSLRAGDTAVYYCARGGVPTTIWSGPYYYYMDVWGKGTTVTVSS

SEQ ID NO: 585 - protein, 8 aa, synthetic construct
GFTLSSYD

SEQ ID NO: 586 - protein, 7 aa, synthetic construct
IGTSGDT

SEQ ID NO: 587 - protein, 20 aa, synthetic construct
ARGGVPTTIWSGPYYYYMDV

SEQ ID NO: 588 - protein, 112 aa, synthetic construct
QSVLTQPPSVSAAPGQKVTISCSGSSSNIGNNYVSWYQQLPGTAPKLLIYENNKRPSGIPDRFSGSKSGTSATLGITGLQTGDEADYYCGTWDSSLSAGQGVFGGGTKLTVL

SEQ ID NO: 589 - protein, 8 aa, synthetic construct
SSNIGNNY

SEQ ID NO: 590 - skipped

SEQ ID NO: 591 - protein, 13 aa, synthetic construct
GTWDSSLSAGQGV

SEQ ID NO: 592 - protein, 118 aa, synthetic construct
QVQLVQSGPEVKKPGASVKVSCKASGYTFTSYAISWVRQAPGQGLEWMGWISAYNGNTNYAQKLQGRVTMTTDTFTSTAYMELRSLRSDDTAVYYCAVPADHITFDYWGQGTLVTVSS

SEQ ID NO: 593 - protein, 8 aa, synthetic construct
GYTFTSYA

SEQ ID NO: 594 - protein, 8 aa, synthetic construct
ISAYNGNT

SEQ ID NO: 595 - protein, 11 aa, synthetic construct
AVPADHITFDY

SEQ ID NO: 596 - protein, 107 aa, synthetic construct
EIVLTQSPGTLSLSPGERATLSCRASQSVSSYLAWYQQKPGQAPRLLIHGASSRATGIPDRFSGSGSGTDFTLTISRLEPEDFAVYYCQQYGSSFLTFGGGTKVEIK

SEQ ID NO: 597 - protein, 6 aa, synthetic construct
QSVSSY

SEQ ID NO: 598 - protein, 9 aa, synthetic construct
QQYGSSFLT

SEQ ID NO: 599 - protein, 122 aa, synthetic construct
QVQLVQSGAEVKKPGSSVKVSCKASGGSFSNYAISWVRQAPGQGLEWMGGIIPFFGTTNYAQKFQGRLTITADESTSTFYMELSSLGSEDTAVYYCAAGYSSGWYPSWFDPWGQGTLVTVSS

SEQ ID NO: 600 - protein, 8 aa, synthetic construct
GGSFSNYA

SEQ ID NO: 601 - protein, 8 aa, synthetic construct
IIPFFGTT

SEQ ID NO: 602 - protein, 15 aa, synthetic construct
AAGYSSGWYPSWFDP

SEQ ID NO: 603 - protein, 108 aa, synthetic construct
EIVLTQSPGTLSLSPGERATLSCRASQSVNNNYLAWYQQKPGQTPRLLIYGASSRATGIPDRFSGSGSGTDFTLTITRLEPEDFAVYHCQQYGRSPTTFGQGTRLEIK

SEQ ID NO: 604 - protein, 7 aa, synthetic construct
QSVNNNY

SEQ ID NO: 605 - protein, 9 aa, synthetic construct
QQYGRSPTT

SEQ ID NO: 606 - protein, 124 aa, synthetic construct
EVQLVQSGAEVKKPGSTMKISCKISGYIFSDYYMHWVQQAPGKGLEWMGLVDPEDGETRYAEKFQGRVTITADTSTDTAYMELSSLRFEDTAVYYCATVGGSSGPWGETLFDHWGQGSLVTVSS

SEQ ID NO: 607 - protein, 8 aa, synthetic construct
GYIFSDYY

SEQ ID NO: 608 - protein, 8 aa, synthetic construct
VDPEDGET

SEQ ID NO: 609 - protein, 17 aa, synthetic construct
ATVGGSSGPWGETLFDH

SEQ ID NO: 610 - protein, 112 aa, synthetic construct
DIVMTQSPLSLPVTPGEPASISCRSSQSLLHSNGYNYINWYLQKPGQSPQLLMYLGSNRASGVPDRFSGSGSGTDSTLKISRVEAEDAGVYYCMQTRQTPYTFGQGTKLEIK

SEQ ID NO: 611 - protein, 11 aa, synthetic construct
QSLLHSNGYNY

SEQ ID NO: 612 - skipped

SEQ ID NO: 613 - protein, 9 aa, synthetic construct
MQTRQTPYT

SEQ ID NO: 614 - protein, 126 aa, synthetic construct
EVQLVESGGGLVQPGGSLRLSCVASGFAFSTYEMNWVRQAPGKGLEWVSYISSSGSTIYYADSVKGRLTISRDNVKNSLYLQMNSLRVEDTAIYYCARDAIRNNDILTDPATFDPWGQGTLVTVSS

SEQ ID NO: 615 - protein, 8 aa, synthetic construct
GFAFSTYE

SEQ ID NO: 616 - protein, 8 aa, synthetic construct
ISSSGSTI

SEQ ID NO: 617 - protein, 19 aa, synthetic construct
ARDAIRNNDILTDPATFDP

SEQ ID NO: 618 - protein, 112 aa, synthetic construct
DIVMTQSPLSLPVTPGEPASISCRSSQSLLYSNGYNYLDWYLQKPGQSPQLLISLGSNRASGVPDRFSGSGSGTDFTLKISRVEAEDVGVYYCMQALQTPYTFGQGTKLEIK

SEQ ID NO: 619 - protein, 11 aa, synthetic construct
QSLLYSNGYNY

SEQ ID NO: 620 - protein, 9 aa, synthetic construct
MQALQTPYT

SEQ ID NO: 621 - protein, 130 aa, synthetic construct
QVQLVQSGAEVKKPGASVKVSCKASGYTFTNYYMHWVRQAPGQGLEWMGIINPSGGSTSYAQKFLGRVTMTRDTSTTTVYMELSSLRSEDTAVYFCARVENLPTKWDLLRKGFESGMDVWGQGTTVTVSS

SEQ ID NO: 622 - protein, 8 aa, synthetic construct
GYTFTNYY

SEQ ID NO: 623 - protein, 8 aa, synthetic construct
INPSGGST

SEQ ID NO: 624 - protein, 23 aa, synthetic construct
ARVENLPTKWDLLRKGFESGMDV

SEQ ID NO: 625 - protein, 107 aa, synthetic construct
EIVMTQSPATVSVSPGERATLSCRASQSVSSNLAWYQQKPGQAPRLLIYGASTRATGIPASFSGSGSGTEFTLTISSLQSEDFAVYYCQQYNNWPITFGPGTKVDIK

SEQ ID NO: 626 - protein, 9 aa, synthetic construct
QQYNNWPIT

SEQ ID NO: 627 - protein, 128 aa, synthetic construct
QLQLQESGPGLVKPSETLSLTCTVSGGSISSSGHYWGWIRQSPGKGLDWIGSIYYSGSTYYNPSLKSRVTISVDTSKNQFSLKLNSVTAADTAVYFCARLEAYCSGDTCHYFYGLDVWGQGTTVTVSS

SEQ ID NO: 628 - protein, 10 aa, synthetic construct
GGSISSSGHY

SEQ ID NO: 629 - protein, 20 aa, synthetic construct
ARLEAYCSGDTCHYFYGLDV

SEQ ID NO: 630 - protein, 107 aa, synthetic construct
DIQMTQSPSSLSASVGDRVTITCRASQSINTYLNWYQQKPGKAPNLLIDGASSLQSGVPSRFSGSGSGTDFILTISSLRPEDFATYYCQQSYSIPLTFGGGTRVEIK

SEQ ID NO: 631 - protein, 6 aa, synthetic construct
QSINTY

SEQ ID NO: 632 - protein, 9 aa, synthetic construct
QQSYSIPLT

SEQ ID NO: 633 - protein, 117 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDIGITTPYWGQGTLVTVSS

SEQ ID NO: 634 - protein, 8 aa, synthetic construct
GFTFDDYA

SEQ ID NO: 635 - protein, 8 aa, synthetic construct
ISWNSGSI

SEQ ID NO: 636 - protein, 10 aa, synthetic construct
AKDIGITTPY

SEQ ID NO: 637 - protein, 107 aa, synthetic construct
AIQMTQSPSSLSASVGDRVTITCRASQDIRNDLGWYQQKPGKAPKLLIYSASSLQTGVPSRFSGSGSGTDFTLTISSLHPEDFATYYCLQDYNYVWTFGQGTKVEIK

SEQ ID NO: 638 - protein, 6 aa, synthetic construct
QDIRND

SEQ ID NO: 639 - skipped

SEQ ID NO: 640 - protein, 9 aa, synthetic construct
LQDYNYVWT

SEQ ID NO: 641 - protein, 124 aa, synthetic construct
EVQLVESGGGLIQPGRSLRLSCAASGFTFDDYAMHWVRQAPGKGLEWVSGISWNSVSIDYADSVKGRFTISRDNAKNSLYLQMTSLTVEDTALYYCAKAFGYETLTGSDYFAYWGPGTLVTVSS

SEQ ID NO: 642 - protein, 8 aa, synthetic construct
ISWNSVSI

SEQ ID NO: 643 - protein, 17 aa, synthetic construct
AKAFGYETLTGSDYFAY

SEQ ID NO: 644 - protein, 109 aa, synthetic construct
EIVLTQSPGTLSLSPGERATLSCRASQTVISTYLAWYQQKPGQSPRLLIYGASSRATGIPDRFSGSGSGTDFTLTISRLEPEDFAVYYCQQYGSSPQLTFGGGTKVEIK

SEQ ID NO: 645 - protein, 7 aa, synthetic construct
QTVISTY

SEQ ID NO: 646 - protein, 10 aa, synthetic construct
QQYGSSPQLT

SEQ ID NO: 647 - protein, 128 aa, synthetic construct
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARAPGNFDWLFRSDYYYGMDVWGQGTTVTVSS

SEQ ID NO: 648 - protein, 8 aa, synthetic construct
GFTFSSYW

SEQ ID NO: 649 - protein, 8 aa, synthetic construct
IKQDGSEK

SEQ ID NO: 650 - protein, 21 aa, synthetic construct
ARAPGNFDWLFRSDYYYGMDV

SEQ ID NO: 651 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPVTFGQGTRLEIK

SEQ ID NO: 652 - protein, 6 aa, synthetic construct
QSISSY

SEQ ID NO: 653 - protein, 9 aa, synthetic construct
QQSYSTPVT

SEQ ID NO: 654 - protein, 119 aa, synthetic construct
QITLKESGPTLVKPTQTLTLTCTFSGFSLSASGVGVGWIRQPPGKALEWLALIYWDDDKRYSPSLKSRLTITKDTSKNQVVLTMTNMDPVDTATYYCAQHKVATIFDYWGQGTLVTVSS

SEQ ID NO: 655 - protein, 10 aa, synthetic construct
GFSLSASGVG

SEQ ID NO: 656 - protein, 11 aa, synthetic construct
AQHKVATIFDY

SEQ ID NO: 657 - protein, 109 aa, synthetic construct
QSALTQPPSASGSPGQSVTISCTATSSDVGNYNYVSWYQQHPGKAPKLMIYEVTKRPSGVPDRFSGSKSGNTASLTVSGLQAEDEADYYCSSYAGSWGVFGGGTKLTVL

SEQ ID NO: 658 - protein, 9 aa, synthetic construct
SSDVGNYNY

SEQ ID NO: 659 - protein, 9 aa, synthetic construct
SSYAGSWGV

SEQ ID NO: 660 - protein, 119 aa, synthetic construct
QVQLVESGGGVVQPGRSLRLSCAASGFIFSSYGMHWVRQAPGKGLEWVAVIWYDGSNKFYADSVKGRFTISRDNSKNTLYLQVNSLRAEDTAVYYCARAVQGDWYFDHWGQGTLVTVSS

SEQ ID NO: 661 - protein, 8 aa, synthetic construct
GFIFSSYG

SEQ ID NO: 662 - protein, 8 aa, synthetic construct
IWYDGSNK

SEQ ID NO: 663 - protein, 12 aa, synthetic construct
ARAVQGDWYFDH

SEQ ID NO: 664 - protein, 108 aa, synthetic construct
SYELTQPPSVSVSPGQTARITCSGDVLAKLYAYWYQHKPGQAPVLVIYKDSERPSGIPERFSGSSSGTTVTLTISGVQAEDEADYYCQSADSSGSLWVFGGGTKLTVL

SEQ ID NO: 665 - protein, 6 aa, synthetic construct
VLAKLY

SEQ ID NO: 666 - skipped

SEQ ID NO: 667 - protein, 11 aa, synthetic construct
QSADSSGSLWV

SEQ ID NO: 668 - protein, 127 aa, synthetic construct
QVQLVESGGGVVQPGRSLRLSCAASGFTFSSYGIHWVRQAPGKGLEWVAIIWYDGSEKYYADSVKGRFTISRDNSKNTLFLQMNSLEAEDTAVYYCARGPDLLTGYYNYFYYGMDVWGQGTTVTVSS

SEQ ID NO: 669 - protein, 8 aa, synthetic construct
GFTFSSYG

SEQ ID NO: 670 - protein, 8 aa, synthetic construct
IWYDGSEK

SEQ ID NO: 671 - protein, 20 aa, synthetic construct
ARGPDLLTGYYNYFYYGMDV

SEQ ID NO: 672 - protein, 111 aa, synthetic construct
QSVLTQPPSVSGAPGQRVTISCTGSSSNIGIGYDVHWYQQLPGTVPKLLIYGNTNRPSGVPDRFSGSKSGTSASLAITGLQAEDEADYYCQSYDSSLSSYVFGTGTKVTVL

SEQ ID NO: 673 - protein, 9 aa, synthetic construct
SSNIGIGYD

SEQ ID NO: 674 - skipped

SEQ ID NO: 675 - protein, 11 aa, synthetic construct
QSYDSSLSSYV

SEQ ID NO: 676 - protein, 119 aa, synthetic construct
QITLKESGPTLVKPTQTLTLTCTFSGFSLNTGGVGVGWIRQPPGNALEWLALIYWDDDKRYNPSLKTRLTISKDTSKNQVVLTMTNMDPVDTATYFCAHLTIAVIVDFWGQGTLVTVSS

SEQ ID NO: 677 - protein, 10 aa, synthetic construct
GFSLNTGGVG

SEQ ID NO: 678 - protein, 11 aa, synthetic construct
AHLTIAVIVDF

SEQ ID NO: 679 - protein, 109 aa, synthetic construct
QSALTQPASVSGSPGQSITISCTATSSDVGTYNYVSWFQHHPGKAPKLMIYEVTNRPSGVSNRFSGSKSGNTASLTISGLQAEDEADYYCSSYTTTSIVFGGGTKLTVL

SEQ ID NO: 680 - protein, 9 aa, synthetic construct
SSDVGTYNY

SEQ ID NO: 681 - protein, 9 aa, synthetic construct
SSYTTTSIV

SEQ ID NO: 682 - protein, 119 aa, synthetic construct
QITLKESGPTLVKPTQTLTLTCTFSGFSLSTSAVGVGWIRQPPGKALEWLALIYWDDDKRYSPSLKSRLTITKDTSKNQLVLTMTNVDPVDTATYYCAHHTIQLIFDYWGQGTLVTVSS

SEQ ID NO: 683 - protein, 10 aa, synthetic construct
GFSLSTSAVG

SEQ ID NO: 684 - protein, 11 aa, synthetic construct
AHHTIQLIFDY

SEQ ID NO: 685 - protein, 111 aa, synthetic construct
QSALTQPASVSGSPGQSITISCTATSSDVGNYNYVSWYQQHPGKAPKLMIYEVSNRPSGVSNRFSGSKSGNTASLTISGLQAEDEADYYCSSYTSSSTPLVFGGGTKLTVL

SEQ ID NO: 686 - protein, 11 aa, synthetic construct
SSYTSSSTPLV

SEQ ID NO: 687 - protein, 130 aa, synthetic construct
QVQLVQSGAEVKKPGASVKVSCKVSGYTLTELSIHWVRQAPGKGLEWMGGFNPEDAETIYAQKFQGRVTMTEDTSTDTAYMELRALRFEDTAVYYCATAFAVVSVGTGLNYNYYYGMDVWGQGTTVTVSS

SEQ ID NO: 688 - protein, 8 aa, synthetic construct
GYTLTELS

SEQ ID NO: 689 - protein, 8 aa, synthetic construct
FNPEDAET

SEQ ID NO: 690 - protein, 23 aa, synthetic construct
ATAFAVVSVGTGLNYNYYYGMDV

SEQ ID NO: 691 - protein, 112 aa, synthetic construct
DIVMTQTPLSSPVTLGQPASISCRSSQSLVHSDGNTYLSWLQQRPGQPPRLLIYKISNRFSGVPDRFSGSGAGTDFTLKISMVEAEDVGVYYCTQATQFPYTFGQGTNLEIK

SEQ ID NO: 692 - protein, 11 aa, synthetic construct
QSLVHSDGNTY

SEQ ID NO: 693 - skipped

SEQ ID NO: 694 - protein, 9 aa, synthetic construct
TQATQFPYT

SEQ ID NO: 695 - protein, 123 aa, synthetic construct
QVQLVQSGAEVKKPGASVKVSCKASGYTFTGYYMHWVRQAPGQGLEWMGRINPYSGGTNYAQKFQGRVTMTRDTSISTAYMELSRLRSDDTAVYYCASGFEPHDYGDPNLPPWGQGTLVTVSS

SEQ ID NO: 696 - protein, 8 aa, synthetic construct
GYTFTGYY

SEQ ID NO: 697 - protein, 8 aa, synthetic construct
INPYSGGT

SEQ ID NO: 698 - protein, 16 aa, synthetic construct
ASGFEPHDYGDPNLPP

SEQ ID NO: 699 - protein, 108 aa, synthetic construct
DIQMTQSPSSLSASVGDRVTITCRASQSISNYLNWYQQKPGKAPKLLIYAASSLQSGVPSRFSGSGSGTDFTLTISSLQPEDFATYYCQHSYSTPPVTFGGGTKVEIK

SEQ ID NO: 700 - protein, 6 aa, synthetic construct
QSISNY

SEQ ID NO: 701 - protein, 10 aa, synthetic construct
QHSYSTPPVT

SEQ ID NO: 702 - protein, 123 aa, synthetic construct
QVQLQESGPGLVKPSETLSLTCTVSGGSISGYYWSWIRQPPGKGLDWIGYIYYRGNTNYNPSLKSRVTISVDTSNNQFSLKLSSVTAADTAVYYCARYGEGYGDASGYHFDYWGQGTLVTVSS

SEQ ID NO: 703 - protein, 7 aa, synthetic construct
IYYRGNT

SEQ ID NO: 704 - protein, 17 aa, synthetic construct
ARYGEGYGDASGYHFDY

SEQ ID NO: 705 - protein, 110 aa, synthetic construct
QSALTQPASVSGSPGQSITISCTGTSSDVGSYNLVSWYQQHPGKAPKLMIYEDSKRPSGVANPFSGSKSGNTASLTISGLQAEDEADYYCSSYAGSSTWVFGGGTKLTVL

SEQ ID NO: 706 - protein, 121 aa, synthetic construct
QVQLQESGPGLVKPSETLSLTCTVSGGSISSHYWSWIRQPPGKGLEWIGYIYYSGSSNYNPSLKSRVTISVDTSKNQFSLKLNSVTAADTAVYYCARHYDILTGFDWFDPWGQGTLVTVSS

SEQ ID NO: 707 - protein, 8 aa, synthetic construct
GGSISSHY

SEQ ID NO: 708 - protein, 7 aa, synthetic construct
IYYSGSS

SEQ ID NO: 709 - protein, 15 aa, synthetic construct
ARHYDILTGFDWFDP

SEQ ID NO: 710 - protein, 112 aa, synthetic construct
QSVLTQPPSVSGAPGQRVTISCTGSSSNIGTHYDVHWYQQLPGTAPKLLIYGNSNRPSGVPDRFSGSKSGTSASLAITGLQAEDEADYYCQSFDNSLTAPYVFGTGTKVTVL

SEQ ID NO: 711 - protein, 9 aa, synthetic construct
SSNIGTHYD

SEQ ID NO: 712 - protein, 12 aa, synthetic construct
QSFDNSLTAPYV

SEQ ID NO: 713 - protein, 120 aa, synthetic construct
EVQLVESGGGLVQPGGSLRLSCAASGFTVSRSYMNWVRQAPGKGLEWVSVVFTGGTTKHTDSVKGRFTISRDNSMNTLYLQMNSLRPEDTAVYYCARVAIVETTGAFDIWGQGTMVTVSS

SEQ ID NO: 714 - protein, 7 aa, synthetic construct
VFTGGTT

SEQ ID NO: 715 - protein, 14 aa, synthetic construct
ARVAIVETTGAFDI

SEQ ID NO: 716 - protein, 112 aa, synthetic construct
QSVLTQPPSVSGAPGQRVTISCTGTSSNIGADFDVHWYQQFPGTAPKLLIYGHTNRPSGIPDRFSGSKSGTSASLAITGLQAEDEADYYCQSYDSSLSGSVVFGGGTKLTVL

SEQ ID NO: 717 - protein, 9 aa, synthetic construct
SSNIGADFD

SEQ ID NO: 718 - skipped

SEQ ID NO: 719 - protein, 119 aa, synthetic construct
QLQLQESGPGLVKPSETLSLICTGSGDSISSSHYYWGWIRQPPGKGLEWIGSTYFSGSTYYNPSLKSRVSISVDTSKNQVSLKLISVTAADTAVYYCAREMGATLGDYWGPGTLVTVSS

SEQ ID NO: 720 - protein, 10 aa, synthetic construct
GDSISSSHYY

SEQ ID NO: 721 - protein, 7 aa, synthetic construct
TYFSGST

SEQ ID NO: 722 - protein, 11 aa, synthetic construct
AREMGATLGDY

SEQ ID NO: 723 - protein, 111 aa, synthetic construct
QSVLTQPPSASGTPGQRVTISCSGSSSNIGNNYVYWYQQFPGTTPRLLIYRNNERPSGVPDRFSGSKSGTSASLVISGLRSEDEADYYCAAWDDSLSAHYVFGTGTKVTVL

SEQ ID NO: 724 - skipped

SEQ ID NO: 725 - protein, 12 aa, synthetic construct
AAWDDSLSAHYV

SEQ ID NO: 726 - protein, 126 aa, synthetic construct
EVQLVESGGGLVQPGGSLRLSCAASGFPFSTYEMNWVRQAPGKGLEWVSYISSSGNTIYYADSVKGRFTISRDNAKNSLYLQMNTLRAEDTAVYYCARDAVRDHDILTDPATFDPWGQGTLVTVSS

SEQ ID NO: 727 - protein, 8 aa, synthetic construct
GFPFSTYE

SEQ ID NO: 728 - protein, 8 aa, synthetic construct
ISSSGNTI

SEQ ID NO: 729 - protein, 19 aa, synthetic construct
ARDAVRDHDILTDPATFDP

SEQ ID NO: 730 - protein, 112 aa, synthetic construct
DIVMTQSPLSLPVTPGEPASISCRSSQTLLHSNGYNYLDWYLQKPGQSPQLLIHLGSNRASGVPDRFSGSGSVTDFTLKISRVEAEDVGVYYCMQALQTPYTFGQGTKLEIK

SEQ ID NO: 731 - protein, 11 aa, synthetic construct
QTLLHSNGYNY

SEQ ID NO: 732 - protein, 119 aa, synthetic construct
EVQLVESGGGLVQPGGSLRLSCAASGFTFSIYEMNWVRQAPGKGLEWVSYITSSGFTIYYADSVKGRFTISRDNAKNSLYLQMHSLRAEDTAVYYCATSSSSGYYFDYWGQGTLVTVSS

SEQ ID NO: 733 - protein, 8 aa, synthetic construct
GFTFSIYE

SEQ ID NO: 734 - protein, 8 aa, synthetic construct
ITSSGFTI

SEQ ID NO: 735 - protein, 12 aa, synthetic construct
ATSSSSGYYFDY

SEQ ID NO: 736 - protein, 113 aa, synthetic construct; identical to SEQ ID NO: 128

SEQ ID NO: 737 - protein, 12 aa, synthetic construct
QSVLYSSNNKNY

SEQ ID NO: 738 - skipped

SEQ ID NO: 739 - protein, 9 aa, synthetic construct
QQYYSTPPT

SEQ ID NO: 740 - protein, 107 aa, synthetic construct
AIQMTQSPSSLSASVGDRVTITCRASQDIRNDLGWYQQKPGKAPKLLIYSASSLQTGVPSRFSGSGSGTDFTLTISSLHPEDFATYYCLQDYDYTWTFGQGTKVEIK

SEQ ID NO: 741 - protein, 9 aa, synthetic construct
LQDYDYTWT

SEQ ID NO: 742 - protein, 107 aa, synthetic construct
AIQMTQSPSSLSASVGDRVTITCRASQDIRNDLGWYQQKPGKAPKLLIYSASSLQTGVPSRFSGSGSGTDFTLTISSLHPEDFATYYCLQDYWYTWTFGQGTKVEIK

SEQ ID NO: 743 - protein, 9 aa, synthetic construct
LQDYWYTWT

SEQ ID NO: 744 - protein, 126 aa, synthetic construct
EVQLVESGGGLVQPGGSLRLSCAASGFTFSNHWMDWVRQAPGKGLVWVSRINSDGSSTDYADSVRGRFTMSRDNAKNTLYLQMSSLRAEDTAVYYCAREGMIFGAVIIPGWYFDLWGRGALVTVSS

SEQ ID NO: 745 - protein, 8 aa, synthetic construct
GFTFSNHW

SEQ ID NO: 746 - protein, 8 aa, synthetic construct
INSDGSST

SEQ ID NO: 747 - protein, 19 aa, synthetic construct
AREGMIFGAVIIPGWYFDL

SEQ ID NO: 748 - protein, 104 aa, synthetic construct
EIVLTQSPGTLSLSPGERATLSCRASQSVSSSQLAWYQQKPGQAPRLLIYGASSRATGIPDRFSGSGSGTDFTLTISRLEPEDFAVYYCQQYDSFGQGTKLEIK

SEQ ID NO: 749 - protein, 7 aa, synthetic construct
QSVSSSQ

SEQ ID NO: 750 - skipped

SEQ ID NO: 751 - protein, 5 aa, synthetic construct
QQYDS

SEQ ID NO: 752 - protein, 117 aa, synthetic construct
EVQLVESGGGLVQPGGSLRLSCAASGITVSSNYMTWVRQAPGKGLEWVSLIYSGGSTFYADSVRGRFTISRDNSKNTLYLQMNSLRAEDTAVYYCARDLVVYGMDVWGQGTTVTVSS